SEQUENCE LISTING (201 sequences)

SEQ ID NO: 1 - protein, 122 aa, Artificial Sequence
  CD33 ccVH  E11
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSS

SEQ ID NO: 2 - protein, 122 aa, Artificial Sequence
  CD33 VH  E11
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 3 - protein, 5 aa, Artificial Sequence
  CD33 HCDR1  E11
NYGMN

SEQ ID NO: 4 - protein, 17 aa, Artificial Sequence
  CD33 HCDR2  E11
WINTYTGEPTYADKFQG

SEQ ID NO: 5 - protein, 13 aa, Artificial Sequence
  CD33 HCDR3  E11
WSWSDGYYVYFDY

SEQ ID NO: 6 - protein, 113 aa, Artificial Sequence
  CD33 CC VL  E11
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 7 - protein, 113 aa, Artificial Sequence
  CD33 VL  E11
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 8 - protein, 17 aa, Artificial Sequence
  CD33 LCDR1  E11
KSSQSVLDSSTNKNSLA

SEQ ID NO: 9 - protein, 7 aa, Artificial Sequence
  CD33 LCDR2  E11
WASTRES

SEQ ID NO: 10 - protein, 9 aa, Artificial Sequence
  CD33 LCDR3  E11
QQSAHFPIT

SEQ ID NO: 11 - protein, 250 aa, Artificial Sequence
  CD33 HL CC  E11
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 12 - protein, 250 aa, Artificial Sequence
  CD33 HL  E11
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 13 - protein, 505 aa, Artificial Sequence
  CD33 CC E11 HL x I2C HL Bispecific molecule
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 14 - protein, 530 aa, Artificial Sequence
  CD33 E11 HL x I2C HL
MGWSCIILFLVATATGVHSQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 15 - protein, 993 aa, Artificial Sequence
  CD33 CC x I2C-scFc Bispecific HLE molecule
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 5 aa, Artificial Sequence
  EGFRvIIIxCD3-scFc VH CDR1
NYGMH

SEQ ID NO: 17 - protein, 17 aa, Artificial Sequence
  EGFRvIIIxCD3-scFc VH CDR2
VIWYDGSDKYYADSVRG

SEQ ID NO: 18 - protein, 15 aa, Artificial Sequence
  EGFRvIIIxCD3-scFc VH CDR3
DGYDILTGNPRDFDY

SEQ ID NO: 19 - protein, 16 aa, Artificial Sequence
  EGFRvIIIxCD3-scFc VL CDR1
RSSQSLVHSDGNTYLS

SEQ ID NO: 20 - protein, 7 aa, Artificial Sequence
  EGFRvIIIxCD3-scFc VL CDR2
RISRRFS

SEQ ID NO: 21 - protein, 9 aa, Artificial Sequence
  EGFRvIIIxCD3-scFc VL CDR3
MQSTHVPRT

SEQ ID NO: 22 - protein, 124 aa, Artificial Sequence
  EGFRvIII_CCxCD3-scFc VH
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSS

SEQ ID NO: 23 - protein, 112 aa, Artificial Sequence
  EGFRvIII_CCxCD3-scFc VL
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 24 - protein, 251 aa, Artificial Sequence
  EGFRvIII_CCxCD3-scFc scFv
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 25 - protein, 506 aa, Artificial Sequence
  EGFRvIII_CCxCD3-scFc Bispecific molecule
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 26 - protein, 994 aa, Artificial Sequence
  EGFRvIII_CCxCD3-scFc Bispecific HLE molecule
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 5 aa, Artificial Sequence
  MSLN_5 VH CDR1
DYYMT

SEQ ID NO: 28 - protein, 17 aa, Artificial Sequence
  MSLN_5 VH CDR2
YISSSGSTIYYADSVKG

SEQ ID NO: 29 - protein, 16 aa, Artificial Sequence
  MSLN_5 VH CDR3
DRNSHFDYDRNSHFDY

SEQ ID NO: 30 - protein, 11 aa, Artificial Sequence
  MSLN_5 VL CDR1
RASQGINTWLA

SEQ ID NO: 31 - protein, 7 aa, Artificial Sequence
  MSLN_5 VL CDR2
GASGLQS

SEQ ID NO: 32 - protein, 9 aa, Artificial Sequence
  MSLN_5 VL CDR3
QQAKSFPRT

SEQ ID NO: 33 - protein, 117 aa, Artificial Sequence
  MSLN _5 VH
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSS

SEQ ID NO: 34 - protein, 107 aa, Artificial Sequence
  MSLN_5 VL
DIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 35 - protein, 239 aa, Artificial Sequence
  MSLN_5 scFv
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 36 - protein, 494 aa, Artificial Sequence
  MSLN_5xI2C0 bispecific molecule
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 37 - protein, 982 aa, Artificial Sequence
  MSLN_5xCD3-scFc Bispecific HLE molecule
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 982 aa, Artificial Sequence
  MSLN_5_CCxCD3-scFc Bispecific HLE molecule
QVQLVESGGGLVKPGGSLRLSCAASGFTFSDHYMSWIRQAPGKCLEWFSYISSSGGIIYYADSVKGRFTISRDNAKNSLYLQMNSLRAEDTAVYYCARDVGSHFDYWGQGTLVTVSSGGGGSGGGGSGGGGSDIQMTQSPSSVSASVGDRVTITCRASQDISRWLAWYQQKPGKAPKLLISAASRLQSGVPSRFSGSGSGTDFTLTISSLQPEDFAIYYCQQAKSFPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 5 aa, Artificial Sequence
  CDR-H1  CDH19 65254.007
SYGMH

SEQ ID NO: 40 - protein, 17 aa, Artificial Sequence
  CDR-H2  CDH19 65254.007
FIWYEGSNKYYAESVKD

SEQ ID NO: 41 - protein, 16 aa, Artificial Sequence
  CDR-H3  CDH19 65254.007
RAGIIGTIGYYYGMDV

SEQ ID NO: 42 - protein, 11 aa, Artificial Sequence
  CDR-L1  CDH19 65254.007
SGDRLGEKYTS

SEQ ID NO: 43 - protein, 7 aa, Artificial Sequence
  CDR-L2  CDH19 65254.007
QDTKRPS

SEQ ID NO: 44 - protein, 9 aa, Artificial Sequence
  CDR-L3  CDH19 65254.007
QAWESSTVV

SEQ ID NO: 45 - protein, 125 aa, Artificial Sequence
  VH  CDH19 65254.007
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSS

SEQ ID NO: 46 - protein, 107 aa, Artificial Sequence
  VL  CDH19 65254.007
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 47 - protein, 247 aa, Artificial Sequence
  VH-VL  CDH19 65254.007
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 48 - protein, 507 aa, Artificial Sequence
  CDH19 65254.007 x I2C
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 49 - protein, 989 aa, Artificial Sequence
  CDH19 65254.007 x I2C -scFc Bispecific HLE molecule
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 987 aa, Artificial Sequence
  CDH19 65254.007 x I2C -scFc_delGK Bispecific HLE molecule
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 125 aa, Artificial Sequence
  CDH19 65254.007_CC x I2C -scFc VH
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSS

SEQ ID NO: 52 - protein, 106 aa, Artificial Sequence
  CDH19 65254.007_CC x I2C -scFc VL
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVL

SEQ ID NO: 53 - protein, 246 aa, Artificial Sequence
  CDH19 65254.007_CC x I2C -scFc scFv
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVL

SEQ ID NO: 54 - protein, 501 aa, Artificial Sequence
  CDH19 65254.007_CC x I2C -scFc Bispecific molecule
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 55 - protein, 989 aa, Artificial Sequence
  CDH19 65254.007_CC x I2C -scFc Bispecific HLE molecule
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 987 aa, Artificial Sequence
  CDH19 65254.007_CC x I2C -scFc_delGK Bispecific HLE molecule
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 7 aa, Artificial Sequence
  FLT3_7 A8xCD3-scFc VH CDR1
NARMGVS

SEQ ID NO: 58 - protein, 32 aa, Artificial Sequence
  FLT3_7 A8xCD3-scFc VH CDR2
HIFSNDEKSYSTSLKNHIFSNDEKSYSTSLKN

SEQ ID NO: 59 - protein, 28 aa, Artificial Sequence
  FLT3_7 A8xCD3-scFc VH CDR3
IVGYGSGWYGFFDYIVGYGSGWYGFFDY

SEQ ID NO: 60 - protein, 11 aa, Artificial Sequence
  FLT3_7 A8xCD3-scFc VL CDR1
RASQGIRNDLG

SEQ ID NO: 61 - protein, 7 aa, Artificial Sequence
  FLT3_7 A8xCD3-scFc VL CDR2
AASTLQS

SEQ ID NO: 62 - protein, 9 aa, Artificial Sequence
  FLT3_7 A8xCD3-scFc VL CDR3
LQHNSYPLT

SEQ ID NO: 63 - protein, 124 aa, Artificial Sequence
  FLT3_7 A8xCD3-scFc VH
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 64 - protein, 107 aa, Artificial Sequence
  FLT3_ A8-scFc VL
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 65 - protein, 246 aa, Artificial Sequence
  FLT3_7 A8xCD3- scFv
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 66 - protein, 501 aa, Artificial Sequence
  FLT3_7 A8xCD3 Bispecific molecule
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 67 - protein, 989 aa, Artificial Sequence
  FLT3_7 A8xCD3-scFc Bispecific HLE molecule
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 5 aa, Artificial Sequence
  VH CDR1 DLL3_1_CC_delGK
SYYWS

SEQ ID NO: 69 - protein, 16 aa, Artificial Sequence
  VH CDR2 DLL3_1_CC_delGK
YVYYSGTTNYNPSLKS

SEQ ID NO: 70 - protein, 10 aa, Artificial Sequence
  VH CDR3 DLL3_1_CC_delGK
IAVTGFYFDY

SEQ ID NO: 71 - protein, 12 aa, Artificial Sequence
  VL CDR1 DLL3_1_CC_delGK
RASQRVNNNYLA

SEQ ID NO: 72 - protein, 7 aa, Artificial Sequence
  VL CDR2 DLL3_1_CC_delGK
GASSRAT

SEQ ID NO: 73 - protein, 9 aa, Artificial Sequence
  VL CDR3 DLL3_1_CC_delGK
QQYDRSPLT

SEQ ID NO: 74 - protein, 118 aa, Artificial Sequence
  VH DLL3_1_CC_delGK
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 75 - protein, 108 aa, Artificial Sequence
  VL DLL3_1_CC_delGK
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 76 - protein, 241 aa, Artificial Sequence
  DLL3_1_CC_delGK
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 77 - protein, 496 aa, Artificial Sequence
  DLL3_1_CCxCD3_delGK  Bispecific molecule
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 78 - protein, 982 aa, Artificial Sequence
  DLL3_1_CCxCD3-scFc_delGK  Bispecific HLE molecule
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 5 aa, Artificial Sequence
  VH CDR1 CD19 97-G1RE-C2
SYGMH

SEQ ID NO: 80 - protein, 17 aa, Artificial Sequence
  VH CDR2 CD19 97-G1RE-C2
VISYEGSNKYYAESVKG

SEQ ID NO: 81 - protein, 13 aa, Artificial Sequence
  VH CDR3 CD19 97-G1RE-C2
DRGTIFGNYGLEV

SEQ ID NO: 82 - protein, 122 aa, Artificial Sequence
  VH CD19 97-G1RE-C2 CC
QVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSS

SEQ ID NO: 83 - protein, 16 aa, Artificial Sequence
  VL CDR1 CD19 97-G1RE-C2
RSSQSLLHKNAFNYLD

SEQ ID NO: 84 - protein, 7 aa, Artificial Sequence
  VL CDR2 CD19 97-G1RE-C2
LGSNRAS

SEQ ID NO: 85 - protein, 9 aa, Artificial Sequence
  VL CDR3 CD19 97-G1RE-C2
MQALQTPFT

SEQ ID NO: 86 - protein, 112 aa, Artificial Sequence
  VL CD19 97-G1RE-C2 CC
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIK

SEQ ID NO: 87 - protein, 525 aa, Artificial Sequence
  CD19 97-G1RE-C2 CC x I2C0
MDMRVPAQLLGLLLLWLRGARCDIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 88 - protein, 1013 aa, Artificial Sequence
  CD19 97-G1RE-C2 CC x I2C0-scFc
MDMRVPAQLLGLLLLWLRGARCDIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 5 aa, Artificial Sequence
  VH CDR1 CDH3 G8A 6-B12
SYPIN

SEQ ID NO: 90 - protein, 16 aa, Artificial Sequence
  VH CDR2 CDH3 G8A 6-B12
VIWTGGGTNYASSVKG

SEQ ID NO: 91 - protein, 15 aa, Artificial Sequence
  VH CDR3 CDH3 G8A 6-B12
SRGVYDFDGRGAMDY

SEQ ID NO: 92 - protein, 17 aa, Artificial Sequence
  VL CDR1 CDH3 G8A 6-B12
KSSQSLLYSSNQKNYFA

SEQ ID NO: 93 - protein, 7 aa, Artificial Sequence
  VL CDR2 CDH3 G8A 6-B12
WASTRES

SEQ ID NO: 94 - protein, 9 aa, Artificial Sequence
  VL CDR3 CDH3 G8A 6-B12
QQYYSYPYT

SEQ ID NO: 95 - protein, 123 aa, Artificial Sequence
  VH CDH3 G8A 6-B12
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSS

SEQ ID NO: 96 - protein, 113 aa, Artificial Sequence
  VL CDH3 G8A 6-B12
DIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIK

SEQ ID NO: 97 - protein, 251 aa, Artificial Sequence
  CDH3 G8A 6-B12 scFv
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIK

SEQ ID NO: 98 - protein, 506 aa, Artificial Sequence
  CDH3 G8A 6-B12 x I2C0 bispecific molecule
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 99 - protein, 994 aa, Artificial Sequence
  CDH3 G8A 6-B12 x I2C0 bispecific molecule HLE
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 5 aa, Artificial Sequence
  BCMA A7 27-C4-G7 CDR1 VH
NHIIH

SEQ ID NO: 101 - protein, 17 aa, Artificial Sequence
  BCMA A7 27-C4-G7 CDR2 VH
YINPYPGYHAYNEKFQG

SEQ ID NO: 102 - protein, 12 aa, Artificial Sequence
  BCMA A7 27-C4-G7 CDR3 VH
DGYYRDTDVLDY

SEQ ID NO: 103 - protein, 11 aa, Artificial Sequence
  BCMA A7 27-C4-G7 CDR1 VL
QASQDISNYLN

SEQ ID NO: 104 - protein, 7 aa, Artificial Sequence
  BCMA A7 27-C4-G7 CDR2 VL
YTSRLHT

SEQ ID NO: 105 - protein, 9 aa, Artificial Sequence
  BCMA A7 27-C4-G7 CDR3 VL
QQGNTLPWT

SEQ ID NO: 106 - protein, 121 aa, Artificial Sequence
  BCMA A7 27-C4-G7 CC (44/100) VH
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 107 - protein, 107 aa, Artificial Sequence
  BCMA A7 27-C4-G7 CC (44/100) VL
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 108 - protein, 243 aa, Artificial Sequence
  BCMA A7 27-C4-G7 CC (44/100) scFv
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 109 - protein, 498 aa, Artificial Sequence
  BCMA A7 27-C4-G7 CC (44/100) x I2C0 bispecific molecule
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 110 - protein, 986 aa, Artificial Sequence
  BCMA A7 27-C4-G7 CC (44/100) x I2C0-scFc bispecific molecule HLE
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 5 aa, Artificial Sequence
  PM 76-B10.17 CC VH CDR1
DYYMY

SEQ ID NO: 112 - protein, 17 aa, Artificial Sequence
  PM 76-B10.17 CC VH CDR2
IISDAGYYTYYSDIIKG

SEQ ID NO: 113 - protein, 12 aa, Artificial Sequence
  PM 76-B10.17 CC VH CDR3
GFPLLRHGAMDY

SEQ ID NO: 114 - protein, 11 aa, Artificial Sequence
  PM 76-B10.17 CC VL CDR1
KASQNVDANVA

SEQ ID NO: 115 - protein, 7 aa, Artificial Sequence
  PM 76-B10.17 CC VL CDR2
SASYVYW

SEQ ID NO: 116 - protein, 9 aa, Artificial Sequence
  PM 76-B10.17 CC VL CDR3
QQYDQQLIT

SEQ ID NO: 117 - protein, 121 aa, Artificial Sequence
  PM 76-B10.17 CC VH
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSS

SEQ ID NO: 118 - protein, 107 aa, Artificial Sequence
  PM 76-B10.17 CC VL
DIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 119 - protein, 243 aa, Artificial Sequence
  PM 76-B10.17 CC scFv
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 120 - protein, 498 aa, Artificial Sequence
  PM 76-B10.17 CC x I2C0 bispecific molecule
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 121 - protein, 986 aa, Artificial Sequence
  PM 76-B10.17 CC x I2C0-scFc bispecific HLE molecule
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 984 aa, Artificial Sequence
  PM 76-B10.17 CC x I2C0-scFc_delGK bispecific HLE molecule
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 498 aa, Artificial Sequence
  PM 76-B10.17 CC x I2C0 CC (103/43)-scFc bispecific molecule
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 124 - protein, 986 aa, Artificial Sequence
  PM 76-B10.17 CC x I2C0 CC (103/43)-scFc bispecific HLE molecule
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 984 aa, Artificial Sequence
  PM 76-B10.17 CC x I2C0 CC (103/43)-scFc_delGK bispecific HLE molecule
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 5 aa, Artificial Sequence
  PM 76-B10.11 CC VH CDR1
DYYMY

SEQ ID NO: 127 - protein, 17 aa, Artificial Sequence
  PM 76-B10.11 CC VH CDR2
IISDGGYYTYYSDIIKG

SEQ ID NO: 128 - protein, 12 aa, Artificial Sequence
  PM 76-B10.11 CC VH CDR3
GFPLLRHGAMDY

SEQ ID NO: 129 - protein, 11 aa, Artificial Sequence
  PM 76-B10.11 CC VL CDR1
KASQNVDTNVA

SEQ ID NO: 130 - protein, 7 aa, Artificial Sequence
  PM 76-B10.11 CC VL CDR2
SASYVYW

SEQ ID NO: 131 - protein, 9 aa, Artificial Sequence
  PM 76-B10.11 CC VL CDR3
QQYDQQLIT

SEQ ID NO: 132 - protein, 121 aa, Artificial Sequence
  PM 76-B10.11 CC VH
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSS

SEQ ID NO: 133 - protein, 107 aa, Artificial Sequence
  PM 76-B10.11 CC VL
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 134 - protein, 243 aa, Artificial Sequence
  PM 76-B10.11 CC scFv
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 135 - protein, 498 aa, Artificial Sequence
  PM 76-B10.11 CC x I2C0 bispecific molecule
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 136 - protein, 986 aa, Artificial Sequence
  PM 76-B10.11 CC x I2C0-scFc bispecific HLE molecule
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 984 aa, Artificial Sequence
  PM 76-B10.11 CC x I2C0-scFc_delGK bispecific HLE molecule
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 498 aa, Artificial Sequence
  PM 76-B10.11 CC x I2C0 CC (103/43)-scFc bispecific molecule
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 139 - protein, 986 aa, Artificial Sequence
  PM 76-B10.11 CC x I2C0 CC (103/43)-scFc bispecific HLE molecule
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 984 aa, Artificial Sequence
  PM 76-B10.11 CC x I2C0 CC (103/43)-scFc_delGK bispecific HLE molecule
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 5 aa, Artificial Sequence
  PM 76-B10.11 CC x I2C0-scFc VH CDR1
DYYMY

SEQ ID NO: 142 - protein, 17 aa, Artificial Sequence
  PM 76-B10.11 CC x I2C0-scFc VH CDR2
IISDGGYYTYYSDIIKG

SEQ ID NO: 143 - protein, 12 aa, Artificial Sequence
  PM 76-B10.11 CC x I2C0-scFc VH CDR3
GFPLLRHGAMDY

SEQ ID NO: 144 - protein, 11 aa, Artificial Sequence
  PM 76-B10.11 CC x I2C0-scFc VL CDR1
KASQNVDTNVA

SEQ ID NO: 145 - protein, 7 aa, Artificial Sequence
  PM 76-B10.11 CC x I2C0-scFc VL CDR2
SASYVYW

SEQ ID NO: 146 - protein, 9 aa, Artificial Sequence
  PM 76-B10.11 CC x I2C0-scFc VL CDR3
QQYDQQLIT

SEQ ID NO: 147 - protein, 121 aa, Artificial Sequence
  PM 76-B10.11 CC x I2C0-scFc VH
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSS

SEQ ID NO: 148 - protein, 107 aa, Artificial Sequence
  PM 76-B10.11 CC x I2C0-scFc VL
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 149 - protein, 243 aa, Artificial Sequence
  PM 76-B10.11 CC x I2C0-scFc scFv
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 150 - protein, 498 aa, Artificial Sequence
  PM 76-B10.11 CC x I2C0-scFc bispecific molecule
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 151 - protein, 986 aa, Artificial Sequence
  PM 76-B10.11 CC x I2C0-scFc bispecific HLE molecule
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 984 aa, Artificial Sequence
  PM 76-B10.11 CC x I2C0-scFc_delGK bispecific HLE molecule
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 498 aa, Artificial Sequence
  PM 76-B10.11 CC x I2C0 CC (103/43)-scFc bispecific molecule
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 154 - protein, 986 aa, Artificial Sequence
  PM 76-B10.11 CC x I2C0 CC (103/43)-scFc bispecific HLE molecule
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 984 aa, Artificial Sequence
  PM 76-B10.11 CC x I2C0 CC (103/43)-scFc_delGK bispecific HLE molecule
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 5 aa, Artificial Sequence
  CD70_21D_CC VH CDR1
TYAMS

SEQ ID NO: 157 - protein, 17 aa, Artificial Sequence
  CD70_21D_CC VH CDR2
AISGSGGRTFYAESVEG

SEQ ID NO: 158 - protein, 11 aa, Artificial Sequence
  CD70_21D_CC VH CDR3
HDYSNYPYFDY

SEQ ID NO: 159 - protein, 12 aa, Artificial Sequence
  CD70_21D_CC VL CDR1
RASQSVRSTYLA

SEQ ID NO: 160 - protein, 7 aa, Artificial Sequence
  CD70_21D_CC VL CDR2
GASSRAT

SEQ ID NO: 161 - protein, 9 aa, Artificial Sequence
  CD70_21D_CC VL CDR3
QQYGDLPFT

SEQ ID NO: 162 - protein, 120 aa, Artificial Sequence
  CD70_21D_CC VH
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSS

SEQ ID NO: 163 - protein, 108 aa, Artificial Sequence
  CD70_21D_CC VL
EIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIK

SEQ ID NO: 164 - protein, 986 aa, Artificial Sequence
  CD70_21D_CCx12C scFc
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSSGGGGSGGGGSGGGGSEIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 1001 aa, Artificial Sequence
  MSLN_5_CCxCD3-scFc Bispecific HLE molecule
MGWSCIILFLVATATGVHSQVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 986 aa, Artificial Sequence; identical to SEQ ID NO: 151
  Anti-PSMA xI2C0 with cys-clamp, scFc Bispecific molecule HLE PM76-B10.11

SEQ ID NO: 167 - protein, 498 aa, Artificial Sequence
  Anti-PSMA IC20 bispecific molecule PM76-B10.17
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 168 - protein, 5 aa, Artificial Sequence
  Anti-Cldn 18.2 VH CDR1 CL-1 and CL-2
GYYMH

SEQ ID NO: 169 - protein, 17 aa, Artificial Sequence
  Anti-Cldn18.2 VH CDR2
WINPNSGGTKYAQKFQG

SEQ ID NO: 170 - protein, 16 aa, Artificial Sequence
  Anti-Cldn18.2 VH CDR3
DRITVAGTYYYYGMDV

SEQ ID NO: 171 - protein, 11 aa, Artificial Sequence
  Anti-Cldn18.2 VL CDR1
RASQGVNNWLA

SEQ ID NO: 172 - protein, 7 aa, Artificial Sequence
  Anti-Cldn18.2 VL CDR2
TASSLQS

SEQ ID NO: 173 - protein, 9 aa, Artificial Sequence
  Anti-Cldn18.2 VL CDR3
QQANSFPIT

SEQ ID NO: 174 - protein, 125 aa, Artificial Sequence
  Anti-Cldn18.2 VH anti-CL-1
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 175 - protein, 107 aa, Artificial Sequence
  Anti-Cldn18.2 VL CL-1
DIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 176 - protein, 247 aa, Artificial Sequence
  Anti-Cldn18.2 scFv CL-1
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 177 - protein, 502 aa, Artificial Sequence
  Anti-Cldn 18.2xCD3 bispecific molecule CL-1 xI2C
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 178 - protein, 990 aa, Artificial Sequence
  Anti-Cldn 18.2xCD3 Bispecific scFc molecule CL-1 xI2C-scFc
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 125 aa, Artificial Sequence
  Anti-Cldn18.2 VH CL-2
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 180 - protein, 107 aa, Artificial Sequence; identical to SEQ ID NO: 175
  Anti-Cldn18.2 VL CL-2

SEQ ID NO: 181 - protein, 247 aa, Artificial Sequence
  Anti-Cldn18.2 scFv CL-2
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 182 - protein, 502 aa, Artificial Sequence
  Anti-Cldn18.2xCD3 bispecific molecule CL-2xI2C
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 183 - protein, 990 aa, Artificial Sequence
  Anti-Cldn18.2xCD3 Bispecific scFc molecule CL-2xI2C-scFc
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 5 aa, Artificial Sequence
  Anti-MUC17 VH CDR1 MU 8-B7
GYYWS

SEQ ID NO: 185 - protein, 16 aa, Artificial Sequence
  Anti-MUC17 VH CDR2 MU 8-B7
DIDASGSTKYNPSLKS

SEQ ID NO: 186 - protein, 12 aa, Artificial Sequence
  Anti-MUC17 VH CDR3 MU 8-B7
KKYSTVWSYFDN

SEQ ID NO: 187 - protein, 11 aa, Artificial Sequence
  Anti-MUC17 VL CDR1 MU 8-B7
SGDKLGDKYAS

SEQ ID NO: 188 - protein, 7 aa, Artificial Sequence
  Anti-MUC17 VL CDR2 MU 8-B7
QDRKRPS

SEQ ID NO: 189 - protein, 9 aa, Artificial Sequence
  Anti-MUC17 VL CDR3 MU 8-B7
QAWGSSTAV

SEQ ID NO: 190 - protein, 120 aa, Artificial Sequence
  Anti-MUC17 VH MU 8-B7
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSS

SEQ ID NO: 191 - protein, 106 aa, Artificial Sequence
  Anti-MUC17 VL MU 8-B7
SYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVL

SEQ ID NO: 192 - protein, 984 aa, Artificial Sequence
  bispecific molecule MU 8-B7 x I2C0scFc
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 14 aa, Artificial Sequence
  CDR-L1 of I2C
GSSTGAVTSGNYPN

SEQ ID NO: 194 - protein, 7 aa, Artificial Sequence
  CDR-L2 of I2C
GTKFLAP

SEQ ID NO: 195 - protein, 9 aa, Artificial Sequence
  CDR-L3 of I2C
VLWYSNRWV

SEQ ID NO: 196 - protein, 5 aa, Artificial Sequence
  CDR-H1 of I2C
KYAMN

SEQ ID NO: 197 - protein, 19 aa, Artificial Sequence
  CDR-H2 of I2C
RIRSKYNNYATYYADSVKD

SEQ ID NO: 198 - protein, 14 aa, Artificial Sequence
  CDR-H3 of I2C
HGNFGNSYISYWAY

SEQ ID NO: 199 - protein, 125 aa, Artificial Sequence
  VH of I2C
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 200 - protein, 109 aa, Artificial Sequence
  VL of I2C
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 201 - protein, 249 aa, Artificial Sequence
  VH-VL of I2C
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

(19) United States
(12) Patent Application Publication
(10) Pub. No.: US 2022/0378908 A1
(43) Pub. Date: Dec. 1, 2022

(54) COMPOSITIONS AND METHODS FOR MINIMIZING PROTEIN LOSS AT LOW PROTEIN CONCENTRATIONS

(71) Applicant: AMGEN INC., Thousand Oaks, CA (US)

(72) Inventors: Chen Zhu, Thousand Oaks, CA (US); Daniel Gerard Greene, Thousand Oaks, CA (US); Qi Hao, Thousand Oaks, CA (US); Sekhar Kanapuram, Thousand Oaks, CA (US); Michael J. Treuheit, Newbury Park, CA (US)

(21) Appl. No.: 17/771,313

(22) PCT Filed: Oct. 23, 2020
(86) PCT No.: PCT/US20/57065

§ 371 Date: Apr. 22, 2022

Related U.S. Application Data
(60) Provisional application No. 62/926,089, filed on Oct. 25, 2019.

(51) Int. Cl. A61K 39/395; A61K 47/26; A61K 47/10; A61J 1/14; A61J 1/10

(52) U.S. Cl. CPC A61K 39/3955; A61K 47/26; A61K 47/10; A61J 1/1468; A61J 1/10; A61K 2039/54

(57) ABSTRACT

The present invention relates to compositions and methods for minimizing protein loss (e.g., due to adsorption to solid surfaces) at low protein concentrations. Inventions disclosed herein generally relate to the field of compositions comprising proteins, in particular, pharmaceutical compositions comprising therapeutic proteins at low protein concentrations. Inventions disclosed herein also relate to methods of administering the composition to a subject in need thereof.

### CROSS REFERENCE OF RELATED APPLICATION

This application claims the benefit of U.S. Provisional Application No. 62/926,089 filed Oct. 25, 2019, which is incorporated in its entirety by reference herein.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: A-2429-WO-PCT_ST25, date created: Oct. 23, 2020 size: 451,608 bytes).

### FIELD OF THE INVENTION

Inventions disclosed herein generally relate to the field of compositions comprising proteins, in particular, pharmaceutical compositions comprising therapeutic proteins at low protein concentrations. Inventions disclosed herein also relate to methods of administering the composition to a subject in need thereof.

### BACKGROUND OF THE INVENTION

Therapeutic proteins are an important class of therapeutics for treating patients. Protein molecules are surface active and subject to potential adsorption to solid surfaces. Therapeutic proteins in pharmaceutical compositions could be adsorbed to solid surfaces that the proteins come into contact with (e.g., the surfaces of a container containing a pharmaceutical composition), which could lead to protein loss during storage and use. Generally, the concentration of therapeutic proteins in those compositions is high (e.g., 1 mg/mL or higher) such that protein adsorption to solid surfaces does not result in insufficient amount of drug available for administration to patients. However, when the concentration of proteins in compositions is low (e.g., less than 0.1 mg/mL, such as when a composition is diluted before administration to patients), the risk of protein loss can be more pronounced, which could potentially lead to insufficient amount of drug available for patient administration.

Surfactants are generally used in pharmaceutical compositions comprising therapeutic proteins, e.g., to prevent protein aggregation and stabilize proteins. It is unclear whether surfactants can be used to effectively prevent protein loss due to surface adsorption when proteins are present at low concentrations in pharmaceutical compositions (e.g., 0.1 mg/mL or less).

There is a need for protein compositions and methods that minimize protein loss due to adsorption to solid surfaces, especially when the compositions comprising protein at low protein concentrations.

### SUMMARY OF THE INVENTION

Disclosed herein are compositions comprising proteins at low protein concentrations as well as methods of administering the compositions to a subject in need thereof. The compositions and methods disclosed herein have the advantage of minimizing or eliminating protein loss due to protein adsorption to solid surfaces and ensure accurate dosing of therapeutic proteins to patients.

In certain embodiments, disclosed herein is an aqueous composition comprising a protein and a surfactant, wherein the protein is present in the composition at a concentration of between about 0.001 μg/ml and about 100 μg/ml, and the surfactant is present in the composition at a concentration of at least about 0.25× of the critical micelle concentration (CMC) of the surfactant.

In certain embodiments, the protein is a bispecific antibody construct comprising a first binding domain that binds to a target cell surface antigen, a second binding domain that binds to human CD3 on the surface of a T cell, and optionally, a third domain comprising, in an amino to carboxyl order, hinge-CH2 domain-CH3 domain-linker-hinge-CH2 domain-CH3 domain. In certain embodiments, the second binding domain comprises a polypeptide having the sequence of SEQ ID NO: 201. In certain embodiments, the bispecific antibody construct is present at a concentration of between about 0.001 μg/ml and about 50 μg/ml, or between about 0.01 μg/ml to about 50 μg/ml, or between 0.1 μg/ml to about 50 μg/ml, or 0.1 μg/ml to about 10 μg/ml, or 1 μg/ml to about 10 μg/ml.

In certain embodiments, the surfactant is a polysorbate, a poloxamer or triton x-100. In certain embodiments, the surfactant is polysorbate 80, polysorbate 60, polysorbate 40, polysorbate 20, or Triton X-100. In certain embodiments, the surfactant is poloxamer 188 or poloxamer 407. In certain embodiments, the surfactant is present at a concentration of between about 0.25× and about 20× of the CMC, or between about 0.25× and about 10× of the CMC of the surfactant.

In certain embodiments, wherein the composition further comprising a salt, an amino acid, a saccharide or saccharide derivative, or combinations thereof. In certain embodiments, the salt is NaCl. In certain embodiments, the saccharide or saccharide derivative is a monosaccharide, a disaccharide, a cyclic polysaccharide or a sugar alcohol. In certain embodiments, the saccharide is sucrose, trehalose, mannitol or sorbitol. In certain embodiments, the amino acid is lysine.

In certain embodiments, wherein the pH of the composition is between about 3.5 and about 7.5. In certain embodiments, the pH of the composition is between about 4.2 and about 7.0.

In certain embodiments, the composition further comprises a buffer or a preservative. In certain embodiments, the buffer is an acetate buffer, a glutamate buffer, a citrate buffer, a succinate buffer, a tartrate buffer, a fumarate buffer, a maleate buffer, a histidine buffer, or phosphate buffer.

In certain embodiments, wherein each of the first and second binding domains of the bispecific antibody construct comprises a VH region and a VL region. In certain embodiments, the bispecific antibody construct is a single chain antibody construct. In certain embodiments, the bispecific antibody construct comprises a polypeptide having the amino acid sequence selected from SEQ ID NO: 11, SEQ ID NO: 13, SEQ ID NO: 15, SEQ ID NO: 24, SEQ ID NO: 25, SEQ ID NO: 47, SEQ ID NO: 48, SEQ ID NO: 66, SEQ ID NO: 67, SEQ ID NO: 76, SEQ ID NO: 78, SEQ ID NO: 87, SEQ ID NO: 88, SEQ ID NO: 108, SEQ ID NO: 109, SEQ ID NO: 110, SEQ ID NO: 165, SEQ ID NO: 166, SEQ ID NO: 167, SEQ ID NO: 176, SEQ ID NO: 178, and SEQ ID NO: 192. In certain embodiments, the bispecific antibody construct comprises a polypeptide comprising the amino acid sequence of SEQ ID NO: 13, SEQ ID NO: 15, SEQ ID NO: 25, SEQ ID NO: 48, SEQ ID NO: 67, SEQ ID NO: 78, SEQ ID NO: 88, SEQ ID NO: 109, SEQ ID NO: 110, SEQ ID NO: 165, SEQ ID NO: 166, SEQ ID NO: 167, SEQ ID NO: 178 or SEQ ID NO: 192.

In certain embodiments, the composition is contained in a plastic container such as an IV bag or IV tube. In certain embodiments, the container is made of a material comprising polyolefin, polyvinyl chloride (PVC), ethyl vinyl acetate (EVA), or polyurethane. In certain embodiments, the container is made of a material comprising PVC and wherein the PVC is substantially free of di-2-ethylhexyl phthalate(DEHP) or tri-2-ethylhexyltrimellitate (TOTM)

In certain embodiments, disclosed herein is a pharmaceutical preparation comprising an aqueous pharmaceutical composition contained inside a container, wherein the aqueous pharmaceutical composition comprising: a bispecific antibody construct at a concentration of between about 0.001 μg/ml and about 100 μg/ml, and a surfactant at a concentration of at least about 0.25× of CMC of the surfactant, wherein the surfactant has an HLB value of at least 20. In certain embodiments, the surfactant is poloxamer 188 or poloxamer 407. In certain embodiments, the aqueous pharmaceutical composition comprises the bispecific antibody construct at a concentration of between about 0.001 μg/ml and about 50 μg/ml. In certain embodiments, the aqueous pharmaceutical composition comprises the surfactant is at a concentration of between about 0.25× and about 20× of the CMC or between about 0.25× and about 10× of the CMC, of the surfactant.

In certain embodiments, the aqueous pharmaceutical composition further comprising a salt, a buffer, an amino acid, a saccharide or saccharide derivative, or combinations thereof. In certain embodiments, the aqueous pharmaceutical composition has a pH of between about 4.2 and about 7.0.

In certain embodiments, the container is made of a material comprising polyolefin, PVC, EVA or polyurethane (e.g., polyester and polyether).

In certain embodiments, the bispecific antibody construct comprises a polypeptide having the amino acid sequences selected from SEQ ID NO: 11, SEQ ID NO: 13, SEQ ID NO: 15, SEQ ID NO: 24, SEQ ID NO: 25, SEQ ID NO: 47, SEQ ID NO: 48, SEQ ID NO: 66, SEQ ID NO: 67, SEQ ID NO: 76, SEQ ID NO: 78, SEQ ID NO: 87, SEQ ID NO: 88, SEQ ID NO: 108, SEQ ID NO: 109, SEQ ID NO: 110, SEQ ID NO: 165, SEQ ID NO: 166, SEQ ID NO: 167, SEQ ID NO: 176, SEQ ID NO: 178, and SEQ ID NO: 192. In certain embodiments, the bispecific antibody construct comprises a polypeptide comprising the amino acid sequence of SEQ ID NO: 13, SEQ ID NO: 15, SEQ ID NO: 25, SEQ ID NO: 48, SEQ ID NO: 67, SEQ ID NO: 78, SEQ ID NO: 88, SEQ ID NO: 109, SEQ ID NO: 110, SEQ ID NO: 165, SEQ ID NO: 166, SEQ ID NO: 167, SEQ ID NO: 178 or SEQ ID NO: 192.

Also disclosed herein is a method of administering a bispecific antibody construct to a patient comprising: preparing an aqueous pharmaceutical composition in a container, wherein the aqueous pharmaceutical composition comprises the bispecific antibody construct at a concentration of between about 0.001 μg/ml and about 100 μg/ml and a surfactant at a concentration of at least about 0.25× of CMC of the surfactant, and administering the aqueous pharmaceutical composition to the patient, wherein the bispecific antibody construct comprises a polypeptide having the amino acid sequence selected from SEQ ID NO: 11, SEQ ID NO: 13, SEQ ID NO: 15, SEQ ID NO: 24, SEQ ID NO: 25, SEQ ID NO: 47, SEQ ID NO: 48, SEQ ID NO: 66, SEQ ID NO: 67, SEQ ID NO: 76, SEQ ID NO: 78, SEQ ID NO: 87, SEQ ID NO: 88, SEQ ID NO: 108, SEQ ID NO: 109, SEQ ID NO: 110, SEQ ID NO: 165, SEQ ID NO: 166, SEQ ID NO: 167, SEQ ID NO: 176, SEQ ID NO: 178, and SEQ ID NO: 192. In certain embodiments, the bispecific antibody construct comprises a polypeptide comprising the amino acid sequence of SEQ ID NO: 13, SEQ ID NO: 15, SEQ ID NO: 25, SEQ ID NO: 48, SEQ ID NO: 67, SEQ ID NO: 78, SEQ ID NO: 88, SEQ ID NO: 109, SEQ ID NO: 110, SEQ ID NO: 165, SEQ ID NO: 166, SEQ ID NO: 167, SEQ ID NO: 178 or SEQ ID NO: 192. In certain embodiments, the aqueous pharmaceutical composition comprises the bispecific antibody construct is at a concentration of between about 0.001 μg/ml and about 50 μg/ml.

In certain embodiments, the aqueous pharmaceutical composition comprises the surfactant is at a concentration of between about 0.25× and about 20× of the CMC or between about 0.25× and about 10× of the CMC, of the surfactant. In certain embodiments, the surfactant is polysorbate 80, polysorbate 60, polysorbate 40, polysorbate 20, poloxamer 188, poloxamer 407, or Triton X-100.

In certain embodiments, the aqueous pharmaceutical composition further comprising one or more selected from a salt, a buffer, an amino acid, a saccharide or a saccharide derivative, and a preservative. In certain embodiments, the aqueous pharmaceutical composition has a pH of between about 4.2 and about 7.0.

In certain embodiments, the container is made of a material comprising polyolefin, PVC, EVA, polyurethane. In certain embodiments, the surfactant is polysorbate 80, polysorbate 60, polysorbate 40, or polysorbate 20, and wherein the container is made of a material comprising PVC that is substantially free of DEHP or TOTM.

In certain embodiments, the aqueous pharmaceutical composition is prepared by diluting a first composition comprising the bispecific antibody construct with a suitable aqueous solution. In certain embodiments, the first composition is a liquid composition comprising the bispecific antibody construct. In certain embodiments, the first composition is a liquid composition reconstituted from a lyophilized composition comprising the bispecific antibody construct. In certain embodiments, the suitable solution comprises the surfactant at a concentration of at least about 0.25× of CMC of the surfactant. In certain embodiments, the aqueous pharmaceutical composition is prepared by adding the suitable aqueous solution into the container followed by adding an appropriate amount of the first composition into the container.

In certain embodiments, the patient is a cancer patient. In certain embodiments, the administration is IV administration.

### BRIEF DESCRIPTION OF THE DRAWINGS

FIG. 1 shows a diagram of the assay for measuring protein binding to a solid surface.

FIG. 2 shows protein binding to a solid surface in the absence of surfactants.

FIG. 3 shows different the addition of various surfactants prevented protein binding to solid surfaces.

FIG. 4 shows that adding surfactants to the solid surface before adding protein prevents protein binding to surfaces more efficiently.

FIG. 5 shows the impacts of different surfactants at the same concentration on the leaching of di-2-ethylhexyl phthalate (DEHP) from DEHP-containing PVC.

FIG. 6 shows the impacts of different surfactants at same folds of CMC on leaching of DEHP from DEHP-containing PVC.

### DETAILED DESCRIPTION

Inventions disclosed herein are based on the surprising finding that surfactants, when used at concentrations lower than their critical micelle concentration, can stabilize proteins at low concentrations in liquid compositions and effectively prevent protein loss due to adsorption to solid surfaces.

In some embodiments, disclosed herein is an aqueous composition comprising a protein and a surfactant, wherein the protein is present in the composition at a concentration of between about 0.001 μg/ml and about 100 μg/ml, and the surfactant is present in the composition at a concentration of at least about 0.25× of the critical micelle concentration (CMC) of the surfactant.

Surfactants that can be used in the composition can be any surfactant typically used in pharmaceutical compositions. In some embodiments, the surfactant is a non-ionic surfactant. In some embodiments, the surfactant is a polysorbate such as polysorbate 20, polysorbate 40, polysorbate 60, polysorbate 65, polysorbate 80 or polysorbate 85. In some embodiments, the surfactant is polysorbate 20, in other embodiments, the surfactant is polysorbate 80. In some embodiments, the surfactant is a poloxamer such as poloxamer 124 poloxamer 188, poloxamer 237, poloxamer 338, and poloxamer 407. In some embodiments, the surfactant is Triton X-100. Various surfactants are available commercially (e.g., Tween 20, Tween 80, Pluronic F68, and Pluronic F127 etc.).

The surfactant can be present in the composition at a concentration that is at least about 0.25 times (0.25×) the critical micelle concentration (CMC) of that surfactant. In some embodiments, the surfactant is present in the composition at a concentration that is between about 0.25× and about 20× of the CMC of the surfactant. In some embodiments, the surfactant is present in the composition at a concentration that is between about 0.25× and about 15×, or between about 0.25× and about 10×, or between about 0.25× and about 8×, or between about 0.25× and about 6×, or between about 0.25× and about 4, or between about 0.25× and about 2×, or between about 0.25× and about 1× of the CMC of the surfactant.

In some embodiments, the surfactant is present in the composition at a concentration that is between about 0.5× and about 20× of the CMC of the surfactant. In some embodiments, the surfactant is present in the composition at a concentration that is between about 0.5× and about 15×, or between about 0.5× and about 10×, or between about 0.5× and about 8×, or between about 0.5× and about 6×, or between about 0.5× and about 4×, or between about 0.5× and about 2×, or between about 0.5× and about 1× of the CMC of the surfactant.

In some embodiments, the surfactant is present in the composition at a concentration of about 0.25×, about 0.5×, about 1×, about 2×, about 3×, about 4×, about 5×, about 6×, about 7×, about 8×, about 9×, about 10×, about 12×, about 14×, about 16×, about 18×, or about 20× of the CMC of the surfactant. In some embodiments, the surfactant is present in the composition at a concentration of about 1.25×, about 2.5×, about 3.5× about 4.5×, about 5.5×, about 6.5×, about 7.5×, about 8.5× or about 9.5× of the CMC of the surfactant.

As used herein, the term “about,” when used to modify a particular value or a range, is understood to mean that there can be variations in a given value or range, including within 20 percent, e.g., within 10 percent, 5 percent, 4 percent, 3 percent, 2 percent, or 1 percent of the stated value or range.

Critical micelle concentration refers to the concentration of a surfactant above which micelles form, it is a property of a surfactant. The CMC value of a surfactant can be measured by experimental methods well known in the art such as fluorometry, surface tension, conductometry and dynamic light scattering. See e.g., Norman Scholz, Thomas Behnke, Ute Resch-Genger. Journal of Fluorescence 28:465-476 (2018) and Önder Topel, Burçin Acar Çakir, Leyla Budama, Numan Hoda. Journal of Molecular Liquids 177 40-43 (2013). The CMC value of a surfactant can also be measured automatically using devices such as Attention® Sigma 700 or 701.

In some embodiments, the CMC value of each of the surfactants is listed in table 1 below.

TABLE 1
CMC of commonly used surfactants

| | CMC* |
|---|---|
| Surfactant | (w/v %) |
| Polysorbate 20 | 0.007 |
| Polysorbate 80 | 0.002 |
| Poloxamer 188 | 0.4 |
| Poloxamer 407 | 0.004 |
| Triton X-100 | 0.014 |

*For the CMC values listed in the table, see e.g., le Maire M, Champed P, Moller J V. 2000. Interaction of membrane proteins and lipids with solubilizing detergents. Biochim Biophys Acta 1508: 86-111; Suksiriworapong J, Rungvimolsin T, A-gomol A, Junyaprasert V B, Chantasart D. Development and characterization of lyophilized diazepam-loaded polymeric micelles. 2014. AAPS PharmSciTech. 15(1): 52-64; https://www.sigmaaldrich.com/content/dam/sigma-aldrich/docs/Sigma/Product_Information_Sheet/1/t8532pis.pdf

The protein can be present in the composition at a concentration that is between about 0.001 μg/ml and about 100 μg/ml. In some embodiments, the protein is present in the composition at a concentration that is between about 0.001 μg/ml and about 90 μg/ml, or between about 0.001 μg/ml and about 80 μg/ml, or between about 0.001 μg/ml and about 70 μg/ml, or between about 0.001 μg/ml and about 60 μg/ml, or between about 0.001 μg/ml and about 50 μg/ml, or between about 0.001 μg/ml and about 40 μg/ml, between about 0.001 μg/ml and about 30 μg/ml, or between about 0.001 μg/ml and about 20 μg/ml, or between about 0.001 μg/ml and about 10 μg/ml, or between about 0.001 μg/ml and about 5 μg/ml, or between about 0.001 μg/ml and about 1 μg/ml, or between about 0.001 μg/ml and about 0.01 μg/ml.

In some embodiments, the protein is present in the composition at a concentration that is between about 0.01 μg/ml and about 100 μg/ml, or between about 0.01 μg/ml and about 80 μg/ml, or between about 0.01 μg/ml and about 70 μg/ml, or between about 0.01 μg/ml and about 60 μg/ml, or between about 0.01 μg/ml and about 50 μg/ml, or between about 0.01 μg/ml and about 40 μg/ml, between about 0.01 μg/ml and about 30 μg/ml, or between about 0.01 μg/ml and about 20 μg/ml, or between about 0.01 μg/ml and about 10 μg/ml, or between about 0.01 μg/ml and about 5 μg/ml, or between about 0.01 μg/ml and about 1 μg/ml, or between about 0.01 μg/ml and about 0.1 μg/ml, or between about 0.1 μg/ml and about 1 μg/ml, or between about 0.1 μg/ml and about 5 In/mi.

In some embodiments, the protein is present in the composition at a concentration of about 0.001 μg/ml, about 0.005 μg/ml, about 0.01 μg/ml, about 0.05 μg/ml, about 0.1 μg/ml, about 1 μg/ml, about 4 μg/ml, about 8 μg/ml, about 10 μg/ml, about 15 μg/ml, about 20 μg/ml, about 25 μg/ml, about 30 μg/ml, about 35 μg/ml, about 40 μg/ml, about 45 μg/ml, about 50 μg/ml, about 55 μg/ml, about 60 μg/ml, about 65 μg/ml, about 70 μg/ml, about 75 μg/ml, about 80 μg/ml, about 85 μg/ml, about 90 μg/ml, about 95 μg/ml, or about 100 μg/ml.

In the composition disclosed herein, any of the concentration or concentration range for the protein can be combined with any of the concentration or concentration range for the surfactant.

Any protein can be the protein in the composition. In some embodiments, the protein in the composition is a therapeutic protein such as an antigen binding protein or a fusion protein.

As used herein, the term “antigen binding protein” refers to a protein that specifically binds to one or more target antigens. An antigen binding protein includes, but not limited to an antibody (e.g., a monoclonal antibody). An antigen binding protein typically comprises an antigen-binding fragment that specifically binds to an antigen and, optionally, a scaffold or framework portion that allows the antigen-binding fragment to adopt a conformation that promotes binding of the antigen binding protein to the antigen. An “antigen binding fragment” refers to a portion of an antibody that lacks at least some of the amino acids present in a full-length heavy chain and/or light chain, but which is still capable of specifically binding to an antigen. An antigen-binding fragment includes, but is not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)2 fragment, a Fv fragment, a Fd fragment, and a complementarity determining region (CDR) fragment, and can be derived from any mammalian source, such as human, mouse, rat, rabbit, or camelid. Antigen-binding fragments may compete for binding of a target antigen with an intact antibody and the fragments may be produced by the modification of intact antibodies (e.g. enzymatic or chemical cleavage) or synthesized de novo using recombinant DNA technologies or peptide synthesis known in the art.

In some embodiments, the protein is an antigen binding protein that is bispecific. As used herein, the term “bispecific” refers to an antigen binding protein capable of specifically binding to two different antigens or targets or epitopes. As used herein, an “epitope” refers to any determinant capable of being specifically bound by an antigen binding protein, such as an antibody or fragment thereof. In some embodiments, the bispecific antigen binding protein comprises a first domain specifically binds to one antigen or target and a second domain specifically binds to another antigen or target. In some embodiments, the first domain of the bispecific antigen binding protein specifically binds to a target cell surface antigen and the second binding domain of the bispecific antigen binding protein specifically binds to the human CD3, a subunit of the T cell receptor complex on T cells. In some preferred embodiments, the bispecific antigen binding protein is a bispecific T cell engager (BiTE®) antibody construct as described in, e.g., WO2008119567 and WO2017134140.

As used herein, the term “antibody construct” refers to a molecule in which the structure and/or function is/are based on the structure and/or function of an antibody, e.g., of a full-length or whole immunoglobulin molecule and/or is/are drawn from the variable heavy chain (VH) and/or variable light chain (VL) domains of an antibody or fragment thereof. An antibody construct is hence capable of binding to its specific target or antigen. Antibody construct also includes modified fragments of antibodies, such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “multibodies” such as triabodies or tetrabodies, and single domain antibodies such as nanobodies or single variable domain antibodies comprising merely one variable domain, which might be VHH, VH or VL, that specifically bind an antigen or epitope independently of other V regions or domains.

In some embodiments, the bispecific antibody construct comprises a first binding domain and a second binding domain, wherein the first binding domain specifically binds to a first cell surface antigen and the second binding domain specifically binds to human CD3. In some embodiments, the first and the second domain of the bispecific antibody construct is a “bispecific single chain antibody construct”, more preferably a bispecific “single chain Fv” (scFv). In a scFv, VL and VH of an antibody are joined, e.g., by a synthetic linker, as a single protein chain in which the VL and VH regions pair to form a monovalent molecule; see e.g., Huston et al. (1988) Proc. Natl. Acad. Sci USA 85:5879-5883). The linker can be a short peptide of about ten to about 25 amino acids, preferably about 15 to 20 amino acids. The linker is usually rich in glycine for flexibility, as well as serine or threonine for solubility, and can either connect the N-terminus of the VH with the C-terminus of the VL, or vice versa. The scFv retains the specificity of the original immunoglobulin, despite removal of the constant regions and introduction of the linker. The VH and VL regions are arranged in the order VH-VL or VL-VH. It is preferred that the VH-region is positioned N-terminally of a linker sequence, and the VL-region is positioned C-terminally of the linker sequence. In certain embodiments, the first and second domain of the bispecific antibody construct are in a format selected from (scFv)₂, scFv-single domain mAb, diabody and oligomers of any of those formats.

In some embodiments, the bispecific antibody construct further comprises a third domain. In some embodiments, the third domain is a single-chain Fc (scFc) domain. In some embodiments, the scFc domain is a scFc half-life extended (HLE) domain. In some preferred embodiments, the third domain of the bispecific antibody construct is an HLE domain with an amino to carboxyl order: hinge-CH2-CH3-linker-hinge-CH2-CH3.

In some embodiments, the first binding domain of the bispecific antibody construct binds to a first cell surface antigen. In some embodiment, the first cell surface antigen is CD70. CD70 (also known as CD27L or TNFSF7) is a type II integral membrane protein whose normal expression is restricted to a subset of activated T and B cells, mature dendritic cells and thymic medullar epithelial cells.

In some embodiments, the first cell surface antigen is a tumor antigen. The term “tumor antigen” as used herein is understood to refer to those antigens that are presented on tumor cells. These antigens can be presented on the cell surface with an extracellular part, which is often combined with a transmembrane and cytoplasmic part of the molecule. These antigens can sometimes be presented only by tumor cells and not by the normal ones. Tumor antigens can be exclusively expressed on tumor cells or might represent a tumor specific mutation compared to normal cells. In this case, they are called tumor-specific antigens. More common are antigens that are presented by tumor cells and normal cells, and they are called tumor-associated antigens. These tumor-associated antigens can be overexpressed compared to normal cells or are accessible for antibody binding in tumor cells due to the less compact structure of the tumor tissue compared to normal tissue. In some embodiments, the first binding domain binds to tumor antigens selected from CD19, CD33, epidermal growth factor receptor variant iii (EGFRvIII), mesothelin (MSLN), cadherin 19 (CDH19), FMS-like tyrosine kinase 3 (FLT3), delta-like ligand 3 (DLL3), Placental-Cadherin (CDH3), B-cell maturation antigen (BCMA), prostate-specific membrane antigen (PSMA), human mucin 17 (MUC17), and claudin-18 isoform 2 (CLDN18.2). In some embodiments, the tumor antigens are human tumor antigens.

In some preferred embodiments, the bispecific antibody construct comprises a first domain, a second domain and optionally a third domain, wherein the first domain binds to CD70 and the second domain binds to human CD3, and the third domain (if present) is an HLE domain with an amino to carboxyl order: hinge-CH2-CH3-linker-hinge-CH2-CH3. In other preferred embodiments, the bispecific antibody construct comprises a first domain, a second domain and optionally a third domain, wherein the first domain binds to a tumor antigen selected from CD19, CD33, EGFRvIII, MSLN, CDH19, FLT3, DLL3, CDH3, BCMA, PSMA, MUC17 and C:DN18.2, and the second domain binds to human CD3, and the third domain (if present) is a HLE domain with an amino to carboxyl order: hinge-CH2-CH3-linker-hinge-CH2-CH3. In a preferred embodiment, the first and second domain are linked together via a peptide linker, and are linked to the third domain (if present) via a peptide linker. Preferred peptide linker have been described herein above and are characterized by the amino acid sequence Gly-Gly-Gly-Gly-Ser, i.e. Gly4Ser, or polymers thereof, i.e. (Gly4Ser)x, where x is an integer of 1 or greater (e.g. 2, 3, 4, 5, 6, or 7). In some of the preferred embodiments, the second binding domain comprises a polypeptide having the amino acid sequence of SEQ ID NO: 201.

In some embodiments, the first binding domain specifically binds to CD33. In some embodiments, the first binding domain comprises a polypeptide having the amino acid sequence of any one of SEQ ID NOs: 1-15.

In some embodiments, the first binding domain specifically binds to EGFRvIII. In some embodiments, the first binding domain comprises a polypeptide having the amino acid sequence of any one of SEQ ID NOs: 16-26.

In some embodiments, the first binding domain specifically binds to MSLN. In some embodiments, the first binding domain comprises a polypeptide having the amino acid sequence of any one of SEQ ID NOs: 27-38 and 165.

In some embodiments, the first binding domain specifically binds to CDH19. In some embodiments, the first binding domain comprises a polypeptide having the amino acid sequence of any one of SEQ ID NOs: 39-56.

In some embodiments, the first binding domain specifically binds to DLL3. In some embodiments, the first binding domain comprises a polypeptide having the amino acid sequence of SEQ ID NOs: 68-78.

In some embodiments, the first binding domain specifically binds to CD19. In some embodiments, the first binding domain comprises a polypeptide having the amino acid sequence of any one of SEQ ID NOs: 79-88.

In some embodiments, the first binding domain specifically binds to FLT3. In some embodiments, the first binding domain comprises a polypeptide having the amino acid sequence of any one of SEQ ID NOs: 57-67.

In some embodiments, the first binding domain specifically binds to CDH3. In some embodiments, the first binding domain comprises a polypeptide having the amino acid sequence of any one of SEQ ID NOs: 89-99.

In some embodiments, the first binding domain specifically binds to BCMA. In some embodiments, the first binding domain comprises a polypeptide having the amino acid sequence of any one of SEQ ID NOs: 100-110.

In some embodiments, the first binding domain specifically binds to PSMA. In some embodiments, the first binding domain comprises a polypeptide having the amino acid sequence of any one of SEQ ID NOs: 111-155, 166 and 167.

In some embodiments, the first binding domain specifically binds to CD70. In some embodiments, the first binding domain comprises a polypeptide having the amino acid sequence of any one of SEQ ID NOs: 156-164.

In some embodiments, the second binding domain of the bispecific antibody construct specifically binds to the human CD3 epsilon on the surface of a T cell. In some embodiments, the second domain of the bispecific antibody construct specifically binds to an extracellular epitope of the human CD3_{ϵ} chain. In some embodiments, the second domain of the bispecific antibody construct that specifically binds to the human CD3 comprises a VL region comprising CDR-L1 having the amino acid sequence of sequence of SEQ ID NO: 193, CDR-L2 having the amino acid of SEQ ID NO: 194, and CDR-L3 having the amino acid sequence of sequence of SEQ ID NO: 195, and a VH region comprising CDR-H1 having the amino acid sequence of sequence of SEQ ID NO: 196, CDR-H2 having the amino acid sequence of sequence of SEQ ID NO: 197, and CDR-H3 having the amino acid sequence of sequence of SEQ ID NO: 198.

In some embodiments, the second domain of the bispecific antibody construct comprises a VH having the amino acid sequence of SEQ ID NO: 199 and a VL having the amino acid of SEQ ID NO: 200. In some embodiments, the second domain of the bispecific antibody construct comprises a polypeptide having the amino acid sequence of SEQ ID NO: 201.

In some embodiments, the protein is a CD70×CD3 bispecific antibody construct, which comprises a first domain that specifically binds to CD70 and a second domain that specifically binds to CD3. In one embodiment, the first domain specifically binds to CD70 and comprises the CDRs as depicted in SEQ ID NOs: 156 to 161, the second domain specifically binds to CD3 and has the CDRs as depicted in SEQ ID NOs: 193-198. In some embodiments, the bispecific antibody construct further comprises an HLE domain (third domain). In one embodiment, the bispecific antibody construct comprises a VH and a VL, wherein the VH comprises a polypeptide comprising, consisting essentially of, or consisting of the amino acid sequence of SEQ ID NO: 162 and the VL comprises a polypeptide comprising, consisting essentially of, or consisting of the amino acid sequence of SEQ ID NO: 163. In one embodiment, the CD70×CD3 bispecific antibody construct comprises a polypeptide that comprises, consists essentially of, or consists of the amino acid sequence of SEQ ID NO: 164.

In some embodiments, the protein is a BCMA×CD3 bispecific antibody construct, which comprises a first domain that specifically binds to BCMA and a second domain that specifically binds to CD3. In some embodiments, the bispecific antibody further comprises an HLE domain (third domain). In one embodiment, the first domain specifically binds to BCMA and has the CDRs as depicted in SEQ ID NOs: 100 to 105, the second domain specifically binds to CD3 and has the CDRs as depicted in SEQ ID NOs: 193-198. In one embodiment, the BCMA×CD3 bispecific antibody construct comprises a VH and a VL, wherein the VH comprises a polypeptide comprising, consisting essentially of, or consisting of the amino acid sequence of SEQ ID NO: 106 and the VL comprises a polypeptide comprising, consisting essentially of, or consisting of the amino acid sequence of SEQ ID NO: 107. In one embodiment, the BCMA×CD3 bispecific antibody construct comprises a polypeptide that comprises, consists essentially of, or consists of the amino acid sequence of SEQ ID NO: 108. In one embodiment, the BCMA×CD3 bispecific antibody construct comprises a polypeptide that comprises, consists essentially of, or consists of the amino acid sequence of SEQ ID NO: 109. In another embodiment, the BCMA×CD3 bispecific antibody construct comprises a polypeptide that comprises, consists essentially of, or consists of the amino acid sequence of SEQ ID NO: 110.

In some embodiments, the bispecific antibody is a CD33×CD3 bispecific antibody construct, which comprises a first domain that specifically binds to CD33 and a second domain that specifically binds to CD3. In some embodiments, the bispecific antibody construct further comprises an HLE domain (third domain). In one embodiment, the first domain specifically binds to CD33 and has the CDRs as depicted in SEQ ID NOs: 3 to 5 and 8 to 10, the second domain specifically binds to CD3 and has the CDRs as depicted in SEQ ID NOs: 193-198. In one embodiment, the CD33×CD3 bispecific antibody construct comprises a VH and a VL, wherein the VH comprises a polypeptide comprising, consisting essentially of, or consisting of the amino acid sequence of SEQ ID NO: 1 or 2 and the VL comprises a polypeptide comprising, consisting essentially of, or consisting of the amino acid sequence of SEQ ID NO: 6 or 7. In another embodiment, the CD33×CD3 bispecific antibody construct comprises a polypeptide that comprises, consists essentially of, or consists of the amino acid sequence of any one of SEQ ID NO: 11 or 12. In another embodiment, the BCMA×CD3 bispecific antibody construct comprises a polypeptide that comprises, consists essentially of, or consists of the amino acid sequence of SEQ ID NO: 13 or 15.

In some embodiments, the protein is an EGFRvIII×CD3 bispecific antibody construct, which comprises a first domain that specifically binds to EGFRvIII and a second domain that specifically binds to CD3. In some embodiments, the bispecific antibody further comprises an HLE domain (third domain). In one embodiment, the first domain specifically binds to EGFRvIII and has the CDRs as depicted in SEQ ID NOs: 16 to 21, the second domain specifically binds to CD3 and has the CDRs as depicted in SEQ ID NOs: 193-198. In one embodiments, the EGFRvIII×CD3 bispecific antibody construct comprises a VH and a VL, wherein the VH comprises a polypeptide comprising, consisting essentially of, or consisting of the amino acid sequence of SEQ ID NO: 22 and the VL comprises a polypeptide comprising, consisting essentially of, or consisting of the amino acid sequence of SEQ ID NO: 23. In one embodiment, the EGFRvIII×CD3 bispecific antibody construct comprises a polypeptide that comprises, consists essentially of, or consists of the amino acid sequence of SEQ ID NO: 24 or 25. In one embodiment, the EGFRvIII×CD3 bispecific antibody construct comprises a polypeptide that comprises, consists essentially of, or consists of the amino acid sequence of SEQ ID NO: 24 or 26.

In some embodiments, the protein is a MSLN×CD3 bispecific antibody construct, which comprises a first domain that specifically binds to MSLN and a second domain that specifically binds to CD3. In some embodiments, the bispecific antibody construct further comprises an HLE domain (third domain). In one embodiment, the first domain specifically binds to MSLN and has the CDRs as depicted in SEQ ID NOs: 27 to 32, the second domain specifically binds to CD3 and has the CDRs as depicted in SEQ ID NOs: 193 to 198. In one embodiment, the MSLN×CD3 bispecific antibody construct comprises a VH and a VL, wherein the VH comprises a polypeptide comprising, consisting essentially of, or consisting of the amino acid sequence of SEQ ID NO: 33 and the VL comprises a polypeptide comprising, consisting essentially of, or consisting of the amino acid sequence of SEQ ID NO: 34. In one embodiment, the MSLN×CD3 bispecific antibody construct comprises a polypeptide that comprises, consists essentially of, or consists of the amino acid sequence of any one of SEQ ID NOs: 35-38. In one embodiment, the MSLN×CD3 bispecific antibody construct comprises a polypeptide that comprises, consists essentially of, or consists of the amino acid sequence of SEQ ID NO: 165.

In some embodiments, the protein is a CDH19×CD3 bispecific antibody construct, which comprises a first domain that specifically binds to CDH19 and a second domain that specifically binds to CD3. In some embodiments, the bispecific antibody further comprises an HLE domain (third domain). In one embodiment, the first domain specifically binds to CDH19 and has the CDRs as depicted in SEQ ID NOs: 39 to 44, the second domain specifically binds to CD3 and has the CDRs as depicted in SEQ ID NOs: 193 to 198. In one embodiment, the CDH19×CD3 bispecific antibody construct comprises a VH and a VL, wherein the VH comprises a polypeptide comprising, consisting essentially of, or consisting of the amino acid sequence of SEQ ID NO: 45 or 51 and the VL comprises a polypeptide comprising, consisting essentially of, or consisting of the amino acid sequence of SEQ ID NO: 46 or 52. In one embodiment, the CDH19×CD3 bispecific antibody construct comprises a polypeptide that comprises, consists essentially of, or consists of the amino acid sequence of any one of SEQ ID NOs: 47, 48-50, and 53-56. In one embodiment, the CDH19×CD3 bispecific antibody construct comprises a polypeptide that comprises, consists essentially of, or consists of the amino acid sequence of SEQ ID NO: 48.

In some embodiments, the protein is a DLL3×CD3 bispecific antibody, which comprises a first domain that specifically binds to DLL3 and a second domain that specifically binds to CD3. In some embodiments, the bispecific antibody further comprises an HLE domain (third domain). In one embodiment, the first domain specifically binds to DLL3 and has the CDRs as depicted in SEQ ID NOs: 68 to 73, the second domain specifically binds to CD3 and has the CDRs as depicted in SEQ ID NOs: 193 to 198. In one embodiment, the DLL3×CD3 bispecific antibody construct comprises a VH and a VL, wherein the VH comprises a polypeptide comprising, consisting essentially of, or consisting of the amino acid sequence of SEQ ID NO: 74 and the VL comprises a polypeptide comprising, consisting essentially of, or consisting of the amino acid sequence of SEQ ID NO: 75. In one embodiment, the DLL3×CD3 bispecific antibody construct comprises a polypeptide that comprises, consists essentially of, or consists of the amino acid sequence of SEQ ID NO: 76-78. In one embodiment, the DLL3×CD3 bispecific antibody construct comprises a polypeptide that comprises, consists essentially of, or consists of the amino acid sequence of SEQ ID NO: 78.

In some embodiments, the protein is a FLT3×CD3 bispecific antibody construct, which comprises a first domain that specifically binds to FLT3 and a second domain that specifically binds to CD3. In some embodiments, the bispecific antibody further comprises an HLE domain (third domain). In one embodiment, the first domain specifically binds to FLT3 and has the CDRs as depicted in SEQ ID NOs: 57 to 62, the second domain specifically binds to CD3 and has the CDRs as depicted in SEQ ID NOs 193 to 198. In one embodiment, the FLT3×CD3 bispecific antibody construct comprises a VH and a VL, wherein the VH comprises a polypeptide comprising, consisting essentially of, or consisting of the amino acid sequence of SEQ ID NO: 63 and the VL comprises a polypeptide comprising, consisting essentially of, or consisting of the amino acid sequence of SEQ ID NO: 64. In one embodiment, the FTL3×CD3 bispecific antibody construct comprises a polypeptide that comprises, consists essentially of, or consists of the amino acid sequence of any of SEQ ID NO: 65-67. In one embodiment, the FTL3×CD3 bispecific antibody construct comprises a polypeptide that comprises, consists essentially of, or consists of the amino acid sequence of SEQ ID NO: 67.

In some embodiments, the protein is a CDH3×CD3 bispecific antibody construct, which comprises a first domain that specifically binds to CDH3 and a second domain that specifically binds to CD3. In some embodiments, the bispecific antibody construct further comprises an HLE domain (third domain). In one embodiment, the first domain specifically binds to CDH3 and has the CDRs as depicted in SEQ ID NOs: 89 to 94, the second domain specifically binds to CD3 and has the CDRs as depicted in SEQ ID NOs: 193 to 198. In one embodiment, the CDH3×CD3 bispecific antibody construct comprises a VH and a VL, wherein the VH comprises a polypeptide comprising, consisting essentially of, or consisting of the amino acid sequence of SEQ ID NO: 95 and the VL comprises a polypeptide comprising, consisting essentially of, or consisting of the amino acid sequence of SEQ ID NO: 96. In one embodiment, the CDH3×CD3 bispecific antibody construct comprises a polypeptide that comprises, consists essentially of, or consists of the amino acid sequence of any of SEQ ID NO: 97-99. In one embodiment, the CDH3×CD3 bispecific antibody construct comprises a polypeptide that comprises, consists essentially of, or consists of the amino acid sequence of SEQ ID NO: 99.

In some embodiments, the protein is a PSMA×CD3 bispecific antibody construct, which comprises a first domain that specifically binds to PSMA and a second domain that specifically binds to CD3. In some embodiments, the bispecific antibody construct further comprises an HLE domain (third domain). In one embodiment, the first domain specifically binds to PSMA and has the CDRs as depicted in any of SEQ ID NOs: 111-116, the second domain specifically binds to CD3 and has the CDRs as depicted in SEQ ID NOs: 193 to 198. In one embodiment, the first domain binds to PSMA and has the CDRs as depicted in any of SEQ ID NOs: 126-131 and 141-146, the second domain binds to CD3 and has the CDRs as depicted in SEQ ID NOs: 193 to 198. In one embodiment, the PSMA×CD3 bispecific antibody comprises a VH and a VL, wherein the VH comprises a polypeptide comprising, consisting essentially of, or consisting of the amino acid sequence of SEQ ID NO: 117, 132 or 147 and the VL comprises a polypeptide comprising, consisting essentially of, or consisting of the amino acid sequence of SEQ ID NO: 118, 133, or 148. In one embodiment, the PSMA×CD3 bispecific antibody construct comprises a polypeptide that comprises, consists essentially of, or consists of the amino acid sequence of any one of SEQ ID NOs: 119-125, 134-140, and 149-155. In one embodiment, the PSMA×CD3 bispecific antibody construct comprises a polypeptide that comprises, consists essentially of, or consists of the amino acid sequence of any of SEQ ID NOs: 121, 122, 124, 125, 136, 137, 139, 140, 151, 152, 154 and 155. In one embodiment, the PSMA×CD3 bispecific antibody construct comprises a polypeptide that comprises, consists essentially of, or consists of the amino acid sequence of SEQ ID NO: 166 or 167.

In some embodiments, the protein is a Cldn18.2×CD3 bispecific antibody construct, which comprises a first domain that specifically binds to Cldn18.2 and a second domain that specifically binds to CD3. In some embodiments, the bispecific antibody further comprises an HLE domain (third domain). In one embodiment, the first domain specifically binds to Cldn18.2 and has the CDRs as depicted in SEQ ID NOs: 168 to 173, the second domain specifically binds to CD3 and has the CDRs as depicted in SEQ ID NOs 193 to 198. In one embodiment, the Cldn18.2×CD3 bispecific antibody construct comprises a VH and a VL, wherein the VH comprises a polypeptide comprising, consisting essentially of, or consisting of the amino acid sequence of SEQ ID NO: 174 and the VL comprises a polypeptide comprising, consisting essentially of, or consisting of the amino acid sequence of SEQ ID NO: 175. In one embodiment, the Cldn18.2×CD3 bispecific antibody construct comprises a polypeptide that comprises, consists essentially of, or consists of the amino acid sequence of SEQ ID NO: 176 or 178. In one embodiment, the Cldn18.2×CD3 bispecific antibody construct comprises a polypeptide that comprises, consists essentially of, or consists of the amino acid sequence of SEQ ID NO: 178.

In some embodiments, the protein is a MUC17×CD3 bispecific antibody construct, which comprises a first domain that specifically binds to MUC17 and a second domain that specifically binds to CD3. In some embodiments, the bispecific antibody further comprises an HLE domain (third domain). In one embodiment, the first domain specifically binds to MUC17 and has the CDRs as depicted in SEQ ID NOs: 184 to 189, the second domain specifically binds to CD3 and has the CDRs as depicted in SEQ ID NOs 193 to 198. In one embodiment, the MUC17×CD3 bispecific antibody construct comprises a VH and a VL, wherein the VH comprises a polypeptide comprising, consisting essentially of, or consisting of the amino acid sequence of SEQ ID NO: 190 and the VL comprises a polypeptide comprising, consisting essentially of, or consisting of the amino acid sequence of SEQ ID NO: 191. In one embodiment, the MUC17×CD3 bispecific antibody construct comprises a polypeptide that comprises, consists essentially of, or consists of the amino acid sequence of SEQ ID NO: 192.

In certain embodiments, the bispecific antibody construct comprises a polypeptide that comprises, consists essentially or consists of the amino acid sequence of SEQ ID NO: 13, SEQ ID NO: 15, SEQ ID NO: 25, SEQ ID NO: 48, SEQ ID NO: 67, SEQ ID NO: 78, SEQ ID NO: 88, SEQ ID NO: 109, SEQ ID NO: 110, SEQ ID NO: 165, SEQ ID NO: 166, SEQ ID NO: 167, SEQ ID NO: 178 or SEQ ID NO: 192.

The bispecific antibody disclosed herein can be prepared by methods known in the art. For example, the bispecific antibody can be prepared by methods disclosed in WO2008/119657 and WO2017/134140.

Additional Excipients that May be Used in the Composition

In some embodiments, the composition further comprises one or more excipients suitable for pharmaceutical compositions. In some embodiments, the composition further comprises a salt, a buffer, a saccharide or a saccharide derivative, an amino acid, or a preservative, or combinations of two or more of the forgoing. In some embodiments, the composition further comprises a salt, a saccharide or a saccharide derivative, an amino acid, and optionally a preservative. In some embodiments, the composition further comprises a salt, a buffer, a saccharide or a saccharide derivative, and an amino acid. In some embodiments, the composition further comprises a salt, a buffer, a saccharide or a saccharide derivative, an amino acid, and a preservative.

Exemplary salts that may be used in the composition include salts that are suitable to be used in pharmaceutical compositions (e.g., NaCl). Exemplary buffers that may be used in the composition include acetate buffer, glutamate buffer, citrate buffer, succinate buffer, tartrate buffer, fumarate buffer, maleate buffer, histidine buffer, and phosphate buffer. Exemplary saccharides or saccharide derivatives include monosaccharides, disaccharides, cyclic polysaccharides and sugar alcohols, such as sugars (e.g., sucrose and trehalose) and sugar alcohol (e.g., mannitol and sorbitol). Exemplary amino acids include lysine, histidine, arginine, glycine, methionine, and alanine. Exemplary preservatives include benzoates (e.g., benzyl alcohol and sodium benzoate) and sorbates. (e.g., potassium sorbate).

The pH of the composition can be in the range of from about 3.5 to 7.5. In some embodiments, the composition has a pH of from about 4.0 to about 7.0. In some embodiments, the composition has a pH of from about 4.2 to about 7.0, or from about 5.0 to about 7.0, or from about 5.5 to about 7.0, or from about 6.0 to about 7.0, or from about 6.5 to about 7.0, or from about 4.2 to about 6.5, or from about 4.2 to about 6.0, or from about 4.2 to about 5.5, or from about 4.2 to about 5.0, or from about 5.0 to about 6.0, or from about 5.0 to about 6.5, or from about 5.0 to about 6.0, or from about 5.0 to about 5.5, or from about 5.5 to about 6.0, or from about 5.5 to about 6.5, or from about 5.5 to about 6.0, or from about 6.0 to about 6.5. In some embodiments, the pH of the composition is about 3.5, about 4.0, about 4.5, about 5.0, about 5.5, about 6.0, about 6.5, about 7.0, or about 7.5.

The pH of the composition may be achieved by using one or more buffers listed above. In some embodiments, the composition comprising a buffer selected from an acetate buffer, a glutamate buffer, a citrate buffer, a succinate buffer, a tartrate buffer, a fumarate buffer, a maleate buffer, a histidine buffer, and a phosphate buffer. In some embodiments, the composition comprising a combination of two buffers selected from the above list of buffers, e.g., a glutamate buffer and a citrate buffer.

In some embodiments, the composition comprises substantially no additional buffer. As used herein, the phrase “substantially no additional buffer” refers that the composition contains no buffering agent added therein for the purpose of adjusting and/or maintaining the pH of the composition. For example, saline solution (0.9% NaCl solution) contains no additional buffer and has a pH of about 5.5. It is believed that the pH of saline solution is achieved and maintained in part by atmospheric CO₂ dissolved in water. See e.g., Reddi, B. AJ Int. J. Med. Sci. 10: 747-750 (2013). Such a composition may contain residual buffer that does not contribute to the buffering capacity of the composition.

In some embodiments, the composition comprises a protein at any of the concentration disclosed above, a surfactant at any of the concentration disclosed above, and further comprises a salt (e.g., NaCl), a sugar (e.g., sucrose), an amino acid (e.g., lysine), and optionally a preservative (e.g., benzyl alcohol). In some embodiments, the composition comprises a protein at any of the concentration disclosed above, a surfactant at any of the concentration disclosed above, and further comprises a salt (e.g., NaCl), a sugar (e.g., sucrose), an amino acid (e.g., lysine), a buffer (e.g., a glutamate and/or citrate buffer), and optionally a preservative (e.g., benzyl alcohol). The pH of the composition can be any of the pH value disclosed above. In some embodiments, the pH of the composition is about 5.5.

In some embodiments, the composition disclosed herein is a pharmaceutical composition. As used herein, the term “pharmaceutical composition” is understood to refer to a formulation comprising a protein (e.g., a bispecific antibody construct) suitable for injection and/or administration into a patient (e.g., a human) in need thereof. More particularly, a pharmaceutical composition is substantially sterile and does not contain any agents that are unduly toxic or infectious to the recipient.

### Containers for the Composition

In some embodiments, the composition disclosed herein is contained in a container. Containers that may be used herein include those suitable for pharmaceutical use, e.g., containers made of materials that are nontoxic and maintain physical integrity. In some embodiments, the container is a component for intravenous (IV) administration. As used herein, the phrase “component for IV administration” is understood to refer to a container or a part of a system that may contact the composition during IV administration. In some embodiments, the container is an IV bag or IV tubing.

Materials that may be used for making containers include those typically used for making pharmaceutical containers such as glass and plastic.

In some embodiments, the container is a plastic container. In some embodiments, the container is made of a material comprising polyolefin (e.g., polypropylene (PP) and polyethylene (PE)), polyvinyl chloride (PVC), ethyl vinyl acetate (EVA), or polyurethane (e.g., polyester and polyether). In some embodiments, the container is made of a material comprising PVC. In some embodiments, the PVC is substantially free of di-2-ethylhexyl phthalate (DEHP) or tri-2-ethylhexyltrimellitate (TOTM). As used herein, the term “substantially free of” is understood to refer to PVC in which DEHP or TOTM is not used and/or detected. DEHP and TOTM are plasticizers that may be used in making PVC softer therefore could be made into different shapes. In some embodiments, the container is made of a material that does not comprise PVC. In some embodiments, the container is made of a material comprising polyolefin, EVA, or polyurethane (e.g., polyester and polyether). In some embodiments, the container is made of a material comprising PP and/or PE.

Under certain conditions, DEHP or TOTM contained in certain types of PVC plastic can leach from the plastic in the presence of certain concentrations of certain surfactants (e.g., polysorbates, see Example 3). Not wish to be bound by any particular theory, it is believed that the more lipophilic a surfactant, the easier DEHP or TOTM can leach out in the presence of the surfactant. The hydrophilic-lipophilic balance (HLB) of a surfactant is a measure of the degree to which it is hydrophilic or lipophilic, and can be calculated by methods known in the art. See e.g., Griffin, William C. Calculation of HLB Values of Non-Ionic Surfactants, Journal of the Society of Cosmetic Chemists, 5 (4): 249-56 (1954). The HLB value can be used to predict properties of a surfactant, e.g., HLB>10 indicates the surfactant is more water-soluble (lipid-insoluble), while HLB<10 indicates the surfactant is more lipid-soluble (water-insoluble). The HLB value of exemplary surfactants that can be used in the composition disclosed herein include 17 (polysorbate 20), 15 (polysorbate 80) (https://pharmlabs.unc.edu/labs/emulsions/hlb.htm) and 29 (poloxamer 188) (http://www.rumapel.com.ar/cosmetica_miscelaneos/ficha_tecnica/Pluracare%20L-%20F%20Grades.pdf). In embodiments where the container is made of a PVC plastic comprising DEHP or TOTM, the surfactant in the composition preferably have an HLB value of at least 20 (e.g., a poloxamer, see Example 3), more preferably an HLB value of between 20 and 30.

Containers such as IV components (e.g., IV bags and tubes used for IV administration) that are made of the above listed materials are commercially available. For example, various suitable containers are available from manufactures such as Baxter Healthcare Corporation and B. Braun Medical Inc.

### Pharmaceutical Preparation

Also disclosed herein are pharmaceutical preparations comprising the aqueous composition disclosed above. As used herein, the term “pharmaceutical preparation” is understood to refer to a preparation comprising the aqueous composition disclosed herein in a suitable pharmaceutical container prior to administration to a patient (e.g., a human). In some embodiments, disclosed herein is a pharmaceutical preparation comprising an aqueous pharmaceutical composition in a container, wherein the aqueous pharmaceutical composition comprising: a) a protein at a concentration of between about 0.001 μg/ml and about 100 μg/ml, and b) a surfactant at a concentration of at least about 0.25× of the CMC of the surfactant, and wherein the protein is not blinatumomab.

In some embodiments, the surfactant comprised in the composition is a polysorbate. In some embodiments, the surfactant is a polysorbate selected from polysorbate 20, polysorbate 40, polysorbate 60, polysorbate 65, polysorbate 80 and polysorbate 85. In some embodiments, the surfactant is polysorbate 20 or polysorbate 80. In some embodiments, the surfactant is a poloxamer. In some embodiments, the surfactant is poloxamer 188 or poloxamer 407. In some embodiments, the surfactant is Triton X-100.

In some embodiments, disclosed herein is a pharmaceutical preparation comprising an aqueous pharmaceutical composition in a container, wherein the aqueous pharmaceutical composition comprising: a) a bispecific antibody construct at a concentration of between about 0.001 μg/ml and about 100 μg/ml, and b) a surfactant at a concentration of at least about 0.25× of CMC of the surfactant, wherein the surfactant is a poloxamer. In some embodiments, the poloxamer is poloxamer 188 or poloxamer 407.

In some embodiments, the surfactant is present in the composition at a concentration that is between about 0.25× and about 20× of the CMC of the surfactant. In some embodiments, the surfactant is present in the composition at a concentration that is between about 0.25× and about 10× of the CMC of the surfactant. In some embodiments, the surfactant is present in the composition at a concentration that is within any of the concentration ranges disclosed above. In some embodiments, the surfactant is present in the composition at a concentration that is any of the concentration disclosed above. The CMC value of a surfactant can be determined by the methods disclosed above. In some embodiments, the CMC value of certain commonly used surfactants are listed in Table 1.

In some embodiments, the aqueous pharmaceutical composition comprising the protein at a concentration of between about 0.001 μg/ml and about 50 μg/ml. In some embodiments, the composition comprising the protein (e.g., a bispecific antibody construct) at a concentration that is within any of the ranges disclosed above. In some embodiments, the protein (e.g., a bispecific antibody construct) is present in the composition at any of the concentrations disclosed above. Any of the concentration or concentration range for the protein disclosed above can be combined with any of the concentration or concentration range for the surfactant disclosed above.

As disclosed above, the protein can be a therapeutic protein such as an antigen binding protein and a fusion protein. In some embodiments, the protein is a bispecific antigen binding protein. In some embodiments, the protein is a bispecific antibody construct disclosed above. In some embodiments, the protein is a CD70×CD3 bispecific antibody construct. In some embodiments, the protein is a BCMA×CD3 bispecific antibody construct. In some embodiments, the protein is a CD33×CD3 bispecific antibody construct. In some embodiments, the protein is an EGFRvIII×CD3 bispecific antibody construct. In some embodiments, the protein is a MSLN×CD3 bispecific antibody construct. In some embodiments, the protein is a CDH19×CD3 bispecific antibody construct. In some embodiments, the protein is a DLL3×CD3 bispecific antibody construct. In some embodiments, the protein is a FLT3×CD3 bispecific antibody construct. In some embodiments, the protein is a CDH3×CD3 bispecific antibody construct. In some embodiments, the protein is a PSMA×CD3 bispecific antibody construct. In some embodiments, the protein is a Cldn18.2×CD3 bispecific antibody construct. In some embodiments, the protein is a MUC17×CD3 bispecific antibody construct. Each of these bispecific antibody constructs is disclosed above. In certain embodiments, the protein comprises, consists essentially or consists of the amino acid sequence of SEQ ID NO: 13, SEQ ID NO: 15, SEQ ID NO: 25, SEQ ID NO: 48, SEQ ID NO: 67, SEQ ID NO: 78, SEQ ID NO: 88, SEQ ID NO: 109, SEQ ID NO: 110, SEQ ID NO: 165, SEQ ID NO: 166, SEQ ID NO: 167, SEQ ID NO: 178 or SEQ ID NO: 192.

The pH of the composition can be within any of the pH ranges disclosed above. In some embodiments, the pH of the composition can be any pH disclosed above.

In some embodiments, the aqueous pharmaceutical composition further comprises one or more excipients suitable for use in a pharmaceutical composition. In some embodiments, the aqueous pharmaceutical composition further comprises a salt, a buffer, an amino acid, a saccharide or a saccharide derivative, a preservative or combinations thereof. In some embodiments, the aqueous pharmaceutical composition further comprises a salt, an amino acid, a saccharide or a saccharide derivative, a preservative or combinations thereof. Exemplary salts, buffers, amino acids, saccharides or derivatives thereof, and preservatives that may be used in the composition are disclosed above.

In some embodiments, the container is a plastic container. In some embodiments, the container is made of a material comprising polyolefin (e.g., PP and PE), PVC, EVA, or polyurethane (e.g., polyester and polyether). In some embodiments, the container is made of a material comprising PVC. In some embodiments, the PVC is substantially free of DEHP or TOTM. In some embodiments, the container is made of a material that does not comprise PVC.

In some embodiments, the surfactant comprised in the composition is a poloxamer, and the container can be made of a material comprising polyolefin, PVC, EVA, or polyurethane (e.g., polyester and polyether). In some embodiments, the surfactant comprised in the composition is a polysorbate or Triton X-100, and the container can be made of a material comprising polyolefin, PVC that is substantially free of DEHP or TOTM, EVA, or polyurethane (e.g., polyester and polyether).

In some embodiments, the container is a component for intravenous (IV) administration. In some embodiments, the container is an IV bag or IV tubing.

In some embodiments, the pharmaceutical preparation comprises an aqueous pharmaceutical composition in a container, wherein the aqueous pharmaceutical composition comprising a bispecific antibody construct (e.g., any of the bispecific antibody construct disclosed herein), a surfactant (e.g., any of the surfactant disclosed herein), and further comprises a salt (e.g., NaCl), an amino acid (e.g., lysine), a saccharide or a saccharide derivative (e.g., sucrose or mannitol), optionally a preservative (e.g., and benzyl alcohol), and wherein the container is made of a material comprising polyolefin, PVC, EVA, or polyurethane (e.g., polyester and polyether). In some embodiments, the pharmaceutical preparation comprises an aqueous pharmaceutical composition in a container, wherein the aqueous pharmaceutical composition comprising a bispecific antibody construct (e.g., any of the bispecific antibody construct disclosed herein), a surfactant (e.g., any of the surfactant disclosed herein), and further comprises a salt (e.g., NaCl), a buffer (e.g., a glutamate buffer and/or a citrate buffer), an amino acid (e.g., lysine), a saccharide or a saccharide derivative (e.g., sucrose or mannitol), optionally a preservative (e.g., benzyl alcohol), and wherein the container is made of a material comprising polyolefin, PVC, EVA, or polyurethane (e.g., polyester and polyether). The concentration of the bispecific antibody construct can be any of the concentration disclosed above for the protein. The concentration of the surfactant can be any of the concentration disclosed above. The pH of the composition is in the range of between about 3.5 and about 7.0. In some embodiments, the pH of the composition is about 5.5. In some embodiments, the container is an IV bag.

### Methods of Administering the Composition

Also disclosed herein are methods of administering the composition disclosed herein to a patient. In some embodiments, disclosed herein is a method of administering a protein to a patient, the method comprises a) preparing an aqueous pharmaceutical composition in a container, wherein the aqueous pharmaceutical composition comprises a protein (e.g., a bispecific antibody construct disclosed herein) at a concentration of between about 0.001 μg/ml and about 100 μg/ml and a surfactant at a concentration of at least about 0.25× of CMC of the surfactant, and b) administering the aqueous pharmaceutical composition to the patient, wherein the protein is not blinatumomab.

In some embodiments, the aqueous pharmaceutical composition is prepared by diluting a first composition comprising the protein (e.g., a bispecific antibody construct) with a suitable aqueous solution. In some embodiments, the aqueous pharmaceutical composition is prepared by adding the suitable solution into the container followed by adding an appropriate amount of a first composition into the container thereby diluting the first composition comprising the protein.

In some embodiments, the first composition is a liquid composition comprising the protein. In some embodiments, the first composition is a liquid composition reconstituted from a lyophilized composition comprising the protein. In some embodiments, the first composition is a liquid composition reconstituted from a lyophilized composition comprising the protein using sterile water.

In some embodiments, the suitable aqueous solution that is used for diluting the first composition comprises the surfactant at a concentration of at least about 0.25× of the CMC of the surfactant. In some embodiments, the suitable aqueous solution that is used for diluting the first composition comprises the surfactant at a concentration of between about 0.25× and 20×, or between about 0.25× and about 10× of the CMC of the surfactant. In some embodiments, the suitable aqueous solution that is used for diluting the first composition comprises the surfactant at a concentration of 0.25×, about 0.5×, about 1×, about 2×, about 3×, about 4×, about 5×, about 6×, about 7×, about 8×, about 9×, about 10×, about 15× or about 20× of the CMC of the surfactant. In some embodiments, the suitable aqueous solution that is used for diluting the first composition further comprises NaCl. In some embodiments, the suitable aqueous solution that is used for diluting the first composition has a pH of between about 3.5 and about 7.0, or between about 4.0 and about 6.5. In some embodiments, the suitable aqueous solution that is used for diluting the first composition has a pH of about 5.5. In some embodiments, the method further comprising rinsing the container with the suitable aqueous solution that is used for diluting the first composition before the preparing step.

In some embodiments, the surfactant in the aqueous pharmaceutical composition is a polysorbate. In some embodiments, the surfactant is a polysorbate selected from polysorbate 20, polysorbate 40, polysorbate 60, polysorbate 65, polysorbate 80 and polysorbate 85. In some embodiments, the surfactant is polysorbate 20 or polysorbate 80. In some embodiments, the surfactant is a poloxamer. In some embodiments, the surfactant is poloxamer 188 or poloxamer 407. In some embodiments, the surfactant is Triton X-100.

In some embodiments, the surfactant in the aqueous pharmaceutical composition is present in the composition at a concentration that is between about 0.25× and about 20× of the CMC of the surfactant. In some embodiments, the surfactant is present in the aqueous pharmaceutical composition at a concentration that is between about 0.25× and about 10× of the CMC of the surfactant. In some embodiments, the surfactant is present in the aqueous pharmaceutical composition at a concentration that is within any of the concentration ranges disclosed above. In some embodiments, the surfactant is present in the aqueous pharmaceutical composition at a concentration that is any of the concentrations disclosed above. The CMC value of a surfactant can be determined by the methods disclosed above. In some embodiments, the CMC value of certain commonly used surfactants are listed in Table 1.

In some embodiments, the aqueous pharmaceutical composition comprising the protein at a concentration of between about 0.001 μg/ml and about 50 μg/ml. In some embodiments, the aqueous pharmaceutical composition comprising the protein at a concentration that is within any of the ranges disclosed above. In some embodiments, the protein is present in the composition at any of the concentrations disclosed above.

As disclosed above, the protein can be a therapeutic protein such as an antigen binding protein, a mAb or a fusion protein. In some embodiments, the protein is a bispecific antigen binding protein. In some embodiments, the protein is a bispecific antibody construct disclosed above. In some embodiments, the protein is a CD70×CD3 bispecific antibody construct. In some embodiments, the protein is a BCMA×CD3 bispecific antibody construct. In some embodiments, the protein is a CD33×CD3 bispecific antibody construct. In some embodiments, the protein is an EGFRvIII×CD3 bispecific antibody construct. In some embodiments, the protein is a MSLN×CD3 bispecific antibody construct. In some embodiments, the protein is a CDH19×CD3 bispecific antibody construct. In some embodiments, the protein is a DLL3×CD3 bispecific antibody construct. In some embodiments, the protein is a FLT3×CD3 bispecific antibody construct. In some embodiments, the protein is a CDH3×CD3 bispecific antibody construct. In some embodiments, the protein is a PSMA×CD3 bispecific antibody construct. In some embodiments, the protein is a Cldn18.2×CD3 bispecific antibody construct. In some embodiments, the protein is a MUC17×CD3 bispecific antibody construct. Each of these bispecific antibody constructs is disclosed above. In certain embodiments, the protein comprises, consists essentially or consists of the amino acid sequence of SEQ ID NO: 13, SEQ ID NO: 15, SEQ ID NO: 25, SEQ ID NO: 48, SEQ ID NO: 67, SEQ ID NO: 78, SEQ ID NO: 88, SEQ ID NO: 109, SEQ ID NO: 110, SEQ ID NO: 165, SEQ ID NO: 166, SEQ ID NO: 167, SEQ ID NO: 178 or SEQ ID NO: 192.

The pH of the composition can be within any of the pH ranges disclosed above. In some embodiments, the pH of the composition can be any pH disclosed above.

In some embodiments, the aqueous pharmaceutical composition further comprises one or more excipients suitable for use in a pharmaceutical composition. In some embodiments, the aqueous pharmaceutical composition further comprises a salt, a buffer, an amino acid, a saccharide or a saccharide derivative, optionally a preservative, or combinations of two or more of the forgoing. In some embodiments, the aqueous pharmaceutical composition further comprises a salt, an amino acid, a saccharide or a saccharide derivative, optionally a preservative, or combinations of two or more of the forgoing. Exemplary salts, buffers, amino acids, saccharides or derivatives thereof, and preservatives that may be used in the composition are disclosed above.

In some embodiments, the container is a plastic container or component. In some embodiments, the container is made of a material comprising polyolefin (e.g., PP and PE), PVC, EVA, or polyurethane (e.g., polyester and polyether). In some embodiments, the container is made of a material comprising PVC. In some embodiments, the PVC is substantially free of DEHP or TOTM. In some embodiments, the container is made of a material that does not comprise PVC. In some embodiments, the container is an IV bag or IV tube.

In some embodiments, the aqueous pharmaceutical composition is administered to the patient via IV administration. In some embodiments, the patient is a cancer patient. In some embodiments, the patient is a human.

### Method for Accessing Binding of Protein to a Solid Surface

Also disclosed herein is a method for accessing binding of proteins to solid surfaces. In some embodiments, disclosed herein is of accessing binding of a protein to a solid surface, comprising: a) incubating an aqueous solution comprising the protein with the solid surface, wherein the protein is labeled with a fluorophore, b) removing the aqueous solution from the solid surface and rinse the surface, and c) imaging the solid surface using confocal microscopy.

Table 2 below lists the sequences disclosed herein

| | Designation | | | AMINO ACID SEQUENCE |
|---|---|---|---|---|
| 1. | CD33 ccVH | artificial | aa | QVQLVQSGAEVKKPGESVKVSCKASGYTFTNYGM |
| | E11 | | | NWVKQAPGQCLEWMGWINTYTGEPTYADKFQG |
| | | | | RVTMTTDTSTSTAYMEIRNLGGDDTAVYYCARWS |
| | | | | WSDGYYVYFDYWGQGTSVTVSS |
| 2. | CD33VH E11 | artificial | aa | QVQLVQSGAEVKKPGESVKVSCKASGYTFTNYGM |
| | | | | NWVKQAPGQGLEWMGWINTYTGEPTYADKFQG |
| | | | | RVTMTTDTSTSTAYMEIRNLGGDDTAVYYCARWS |
| | | | | WSDGYYVYFDYWGQGTSVTVSS |
| 3. | CD33 HCDR1 | artificial | aa | NYGMN |
| | E11 | | | |
| 4. | CD33 HCDR2 | artificial | aa | WINTYTGEPTYADKFQG |
| | E11 | | | |
| 5. | CD33 HCDR3 | artificial | aa | WSWSDGYYVYFDY |
| | E11 | | | |
| 6. | CD33 CCVL | artificial | aa | DIVMTQSPDSLTVSLGERTTINCKSSQSVLDSSTNK |
| | E11 | | | NSLAWYQQKPGQPPKLLLSWASTRESGIPDRFSGS |
| | | | | GSGTDFTLTIDSPQPEDSATYYCQQSAHFPITFGCG |
| | | | | TRLEIK |
| 7. | CD33VL E11 | artificial | aa | DIVMTQSPDSLTVSLGERTTINCKSSQSVLDSSTNK |
| | | | | NSLAWYQQKPGQPPKLLLSWASTRESGIPDRFSGS |
| | | | | GSGTDFTLTIDSPQPEDSATYYCQQSAHFPITFGQG |
| | | | | TRLEIK |
| 8. | CD33 LCDR1 | artificial | aa | KSSQSVLDSSTNKNSLA |
| | E11 | | | |
| 9. | CD33 LCDR2 | artificial | aa | WASTRES |
| | E11 | | | |
| 10. | CD33 LCDR3 | artificial | aa | QQSAHFPIT |
| | E11 | | | |
| 11. | CD33 HLCC | artificial | aa | QVQLVQSGAEVKKPGESVKVSCKASGYTFTNYGM |
| | E11 | | | NWVKQAPGQCLEWMGWINTYTGEPTYADKFQG |
| | | | | RVTMTTDTSTSTAYMEIRNLGGDDTAVYYCARWS |
| | | | | WSDGYYVYFDYWGQGTSVTVSSGGGGSGGGGSG |
| | | | | GGGSDIVMTQSPDSLTVSLGERTTINCKSSQSVLDS |
| | | | | STNKNSLAWYQQKPGQPPKLLLSWASTRESGIPDR |
| | | | | FSGSGSGTDFTLTIDSPQPEDSATYYCQQSAHFPITF |
| | | | | GCGTRLEIK |
| 12. | CD33 HL E11 | artificial | aa | QVQLVQSGAEVKKPGESVKVSCKASGYTFTNYGM |
| | | | | NWVKQAPGQGLEWMGWINTYTGEPTYADKFQG |
| | | | | RVTMTTDTSTSTAYMEIRNLGGDDTAVYYCARWS |
| | | | | WSDGYYVYFDYWGQGTSVTVSSGGGGSGGGGSG |
| | | | | GGGSDIVMTQSPDSLTVSLGERTTINCKSSQSVLDS |
| | | | | STNKNSLAWYQQKPGQPPKLLLSWASTRESGIPDR |
| | | | | FSGSGSGTDFTLTIDSPQPEDSATYYCQQSAHFPITF |
| | | | | GQGTRLEIK |
| 13. | CD33 CCE11 | artificial | aa | QVQLVQSGAEVKKPGESVKVSCKASGYTFTNYGM |
| | HLx I2C HL | | | NWVKQAPGQCLEWMGWINTYTGEPTYADKFQG |
| | Bispecific | | | RVTMTTDTSTSTAYMEIRNLGGDDTAVYYCARWS |
| | molecule | | | WSDGYYVYFDYWGQGTSVTVSSGGGGSGGGGSG |
| | | | | GGGSDIVMTQSPDSLTVSLGERTTINCKSSQSVLDS |
| | | | | STNKNSLAWYQQKPGQPPKLLLSWASTRESGIPDR |
| | | | | FSGSGSGTDFTLTIDSPQPEDSATYYCQQSAHFPITF |
| | | | | GCGTRLEIKSGGGGSEVQLVESGGGLVQPGGSLKL |
| | | | | SCAASGFTFNKYAMNWVRQAPGKGLEWVARIRS |
| | | | | KYNNYATYYADSVKDRFTISRDDSKNTAYLQMNNL |
| | | | | KTEDTAVYYCVRHGNFGNSYISYWAYWGQGTLVT |
| | | | | VSSGGGGSGGGGSGGGGSQTVVTQEPSLTVSPGG |
| | | | | TVTLTCGSSTGAVTSGNYPNWVQQKPGQAPRGLI |
| | | | | GGTKFLAPGTPARFSGSLLGGKAALTLSGVQPEDEA |
| | | | | EYYCVLWYSNRWVFGGGTKLTVL |
| 14. | CD33 E11 HL | artificial | aa | MGWSCIILFLVATATGVHSQVQLVQSGAEVKKPGE |
| | x 12C HL | | | SVKVSCKASGYTFTNYGMNWVKQAPGQGLEWM |
| | | | | GWINTYTGEPTYADKFQGRVTMTTDTSTSTAYMEI |
| | | | | RNLGGDDTAVYYCARWSWSDGYYVYFDYWGQG |
| | | | | TSVTVSSGGGGSGGGGSGGGGSDIVMTQSPDSLT |
| | | | | VSLGERTTINCKSSQSVLDSSTNKNSLAWYQQKPG |
| | | | | QPPKLLLSWASTRESGIPDRFSGSGSGTDFTLTIDSP |
| | | | | QPEDSATYYCQQSAHFPITFGQGTRLEIKSGGGGSE |
| | | | | VQLVESGGGLVQPGGSLKLSCAASGFTFNKYAMN |
| | | | | WVRQAPGKGLEWVARIRSKYNNYATYYADSVKDR |
| | | | | FTISRDDSKNTAYLQMNNLKTEDTAVYYCVRHGNF |
| | | | | GNSYISYWAYWGQGTLVTVSSGGGGSGGGGSGG |
| | | | | GGSQTVVTQEPSLTVSPGGTVTLTCGSSTGAVTSG |
| | | | | NYPNWVQQKPGQAPRGLIGGTKFLAPGTPARFSG |
| | | | | SLLGGKAALTLSGVQPEDEAEYYCVLWYSNRWVFG |
| | | | | GGTKLTVLHHHHHH |
| 15. | CD33 CCx I2C- | artificial | aa | WVKQAPGQCLEWMGWINTYTGEPTYADKFQGRVT |
| | scFc Bispecific | | | MTTDTSTSTAYMEIRNLGGDDTAVYYCARWSWSDG |
| | HLE molecule | | | YYVYFDYWGQGTSVTVSSGGGGSGGGGSGGGGSDI |
| | | | | VMTQSPDSLTVSLGERTTINCKSSQSVLDSSTNKNSL |
| | | | | AWYQQKPGQPPKLLLSWASTRESGIPDRFSGSGSGT |
| | | | | DFTLTIDSPQPEDSATYYCQQSAHFPITFGCGTRLEIKS |
| | | | | GGGGSEVQLVESGGGLVQPGGSLKLSCAASGFTFNK |
| | | | | YAMNWVRQAPGKGLEWVARIRSKYNNYATYYADS |
| | | | | VKDRFTISRDDSKNTAYLQMNNLKTEDTAVYYCVRH |
| | | | | GNFGNSYISYWAYWGQGTLVTVSSGGGGSGGGGS |
| | | | | GGGGSQTVVTQEPSLTVSPGGTVTLTCGSSTGAVTS |
| | | | | GNYPNWVQQKPGQAPRGLIGGTKFLAPGTPARFSG |
| | | | | SLLGGKAALTLSGVQPEDEAEYYCVLWYSNRWVFGG |
| | | | | GTKLTVLGGGGDKTHTCPPCPAPELLGGPSVFLFPPK |
| | | | | PKDTLMISRTPEVTCVVVDVSHEDPEVKFNWYVDGV |
| | | | | EVHNAKTKPCEEQYGSTYRCVSVLTVLHQDWLNGKE |
| | | | | YKCKVSNKALPAPIEKTISKAKGQPREPQVYTLPPSRE |
| | | | | EMTKNQVSLTCLVKGFYPSDIAVEWESNGQPENNYK |
| | | | | TTPPVLDSDGSFFLYSKLTVDKSRWQQGNVFSCSVM |
| | | | | HEALHNHYTQKSLSLSPGKGGGGSGGGGSGGGGSG |
| | | | | GGGSGGGGSGGGGSDKTHTCPPCPAPELLGGPSVFL |
| | | | | FPPKPKDTLMISRTPEVTCVVVDVSHEDPEVKFNWY |
| | | | | VDGVEVHNAKTKPCEEQYGSTYRCVSVLTVLHQDW |
| | | | | LNGKEYKCKVSNKALPAPIEKTISKAKGQPREPQVYTL |
| | | | | PPSREEMTKNQVSLTCLVKGFYPSDIAVEWESNGQP |
| | | | | ENNYKTTPPVLDSDGSFFLYSKLTVDKSRWQQGNVF |
| | | | | SCSVMHEALHNHYTQKSLSLSPGK |
| 16. | EGFRVIIIxCD3- | artificial | aa | NYGMH |
| | scFc VH CDR1 | | | |
| 17. | EGFRvIIIxCD3- | artificial | aa | VIWYDGSDKYYADSVRG |
| | scFc VH CDR2 | | | |
| 18. | EGFRvIIIxCD3- | artificial | aa | DGYDILTGNPRDFDY |
| | scFc VH CDR3 | | | |
| 19. | EGFRvIIIxCD3- | artificial | aa | RSSQSLVHSDGNTYLS |
| | scFc VLCDR1 | | | |
| 20. | EGFRvIIIxCD3- | artificial | aa | RISRRFS |
| | scFc VLCDR2 | | | |
| 21. | EGFRvIIIxCD3- | artificial | aa | MQSTHVPRT |
| | scFc VL CDR3 | | | |
| 22. | EGFRvIII_CCxC | artificial | aa | QVQLVESGGGVVQSGRSLRLSCAASGFTFRNYGMH |
| | D3-scFc VH | | | WVRQAPGKCLEWVAVIWYDGSDKYYADSVRGRFTI |
| | | | | SRDNSKNTLYLQMNSLRAEDTAVYYCARDGYDILTG |
| | | | | NPRDFDYWGQGTLVTVSS |
| 23. | EGFRvIII_CCxC | artificial | aa | DTVMTQTPLSSHVTLGQPASISCRSSQSLVHSDGNTY |
| | D3-scFc VL | | | LSWLQQRPGQPPRLLIYRISRRFSGVPDRFSGSGAGT |
| | | | | DFTLEISRVEAEDVGVYYCMQSTHVPRTFGCGTKVEI |
| | | | | K |
| 24. | EGFRvIII_CCxC | artificial | aa | QVQLVESGGGVVQSGRSLRLSCAASGFTFRNYGMH |
| | D3-scFc scFv | | | WVRQAPGKCLEWVAVIWYDGSDKYYADSVRGRFTI |
| | | | | SRDNSKNTLYLQMNSLRAEDTAVYYCARDGYDILTG |
| | | | | NPRDFDYWGQGTLVTVSSGGGGSGGGGSGGGGSD |
| | | | | TVMTQTPLSSHVTLGQPASISCRSSQSLVHSDGNTYL |
| | | | | SWLQQRPGQPPRLLIYRISRRFSGVPDRFSGSGAGTD |
| | | | | FTLEISRVEAEDVGVYYCMQSTHVPRTFGCGTKVEIK |
| 25. | EGFRvIII_CCxC | artificial | aa | QVQLVESGGGVVQSGRSLRLSCAASGFTFRNYGMH |
| | D3-scFc | | | WVRQAPGKCLEWVAVIWYDGSDKYYADSVRGRFTI |
| | Bispecific | | | SRDNSKNTLYLQMNSLRAEDTAVYYCARDGYDILTG |
| | molecule | | | NPRDFDYWGQGTLVTVSSGGGGSGGGGSGGGGSD |
| | | | | TVMTQTPLSSHVTLGQPASISCRSSQSLVHSDGNTYL |
| | | | | SWLQQRPGQPPRLLIYRISRRFSGVPDRFSGSGAGTD |
| | | | | FTLEISRVEAEDVGVYYCMQSTHVPRTFGCGTKVEIK |
| | | | | SGGGGSEVQLVESGGGLVQPGGSLKLSCAASGFTFN |
| | | | | KYAMNWVRQAPGKGLEWVARIRSKYNNYATYYAD |
| | | | | SVKDRFTISRDDSKNTAYLQMNNLKTEDTAVYYCVR |
| | | | | SGGGGSQTVVTQEPSLTVSPGGTVTLTCGSSTGAVTS |
| | | | | GNYPNWVQQKPGQAPRGLIGGTKFLAPGTPARFSG |
| | | | | SLLGGKAALTLSGVQPEDEAEYYCVLWYSNRWVFGG |
| | | | | GTKLTVL |
| 26. | EGFRvIII_CCxC | artificial | aa | QVQLVESGGGVVQSGRSLRLSCAASGFTFRNYGMH |
| | D3-scFc | | | WVRQAPGKCLEWVAVIWYDGSDKYYADSVRGRFTI |
| | Bispecific HLE | | | SRDNSKNTLYLQMNSLRAEDTAVYYCARDGYDILTG |
| | molecule | | | NPRDFDYWGQGTLVTVSSGGGGSGGGGSGGGGSD |
| | | | | TVMTQTPLSSHVTLGQPASISCRSSQSLVHSDGNTYL |
| | | | | SWLQQRPGQPPRLLIYRISRRFSGVPDRFSGSGAGTD |
| | | | | FTLEISRVEAEDVGVYYCMQSTHVPRTFGCGTKVEIK |
| | | | | SGGGGSEVQLVESGGGLVQPGGSLKLSCAASGFTFN |
| | | | | KYAMNWVRQAPGKGLEWVARIRSKYNNYATYYAD |
| | | | | SVKDRFTISRDDSKNTAYLQMNNLKTEDTAVYYCVR |
| | | | | HGNFGNSYISYWAYWGQGTLVTVSSGGGGSGGGG |
| | | | | SGGGGSQTVVTQEPSLTVSPGGTVTLTCGSSTGAVTS |
| | | | | GNYPNWVQQKPGQAPRGLIGGTKFLAPGTPARFSG |
| | | | | SLLGGKAALTLSGVQPEDEAEYYCVLWYSNRWVFGG |
| | | | | GTKLTVLGGGGDKTHTCPPCPAPELLGGPSVFLFPPK |
| | | | | PKDTLMISRTPEVTCVVVDVSHEDPEVKFNWYVDGV |
| | | | | EVHNAKTKPCEEQYGSTYRCVSVLTVLHQDWLNGKE |
| | | | | YKCKVSNKALPAPIEKTISKAKGQPREPQVYTLPPSRE |
| | | | | EMTKNQVSLTCLVKGFYPSDIAVEWESNGQPENNYK |
| | | | | TTPPVLDSDGSFFLYSKLTVDKSRWQQGNVFSCSVM |
| | | | | HEALHNHYTQKSLSLSPGKGGGGSGGGGSGGGGSG |
| | | | | GGGSGGGGSGGGGSDKTHTCPPCPAPELLGGPSVFL |
| | | | | FPPKPKDTLMISRTPEVTCVVVDVSHEDPEVKFNWY |
| | | | | VDGVEVHNAKTKPCEEQYGSTYRCVSVLTVLHQDW |
| | | | | LNGKEYKCKVSNKALPAPIEKTISKAKGQPREPQVYTL |
| | | | | PPSREEMTKNQVSLTCLVKGFYPSDIAVEWESNGQP |
| | | | | ENNYKTTPPVLDSDGSFFLYSKLTVDKSRWQQGNVF |
| | | | | SCSVMHEALHNHYTQKSLSLSPGK |
| 27. | MSLN_5 VH | artificial | aa | DYYMT |
| | CDR1 | | | |
| 28. | MSLN_5 VH | artificial | aa | YISSSGSTIYYADSVKG |
| | CDR2 | | | |
| 29. | MSLN_5 VH | artificial | aa | DRNSHFDY |
| | CDR3 | | | |
| 30. | MSLN_5 VL | artificial | aa | RASQGINTWLA |
| | CDR1 | | | |
| 31. | MSLN_5 VL | artificial | aa | GASGLOS |
| | CDR2 | | | |
| 32. | MSLN_5 VL | artificial | aa | QQAKSFPRT |
| | CDR3 | | | |
| 33. | MSLN_5 VH | artificial | aa | QVQLVESGGGLVKPGGSLRLSCAASGFTFSDYYMT |
| | | | | WIRQAPGKGLEWLSYISSSGSTIYYADSVKGRFTISR |
| | | | | DNAKNSLFLQMNSLRAEDTAVYYCARDRNSHFDY |
| | | | | WGQGTLVTVSS |
| 34. | MSLN_5 VL | artificial | aa | DIQMTQSPSSVSASVGDRVTITCRASQGINTWLA |
| | | | | WYQQKPGKAPKLLIYGASGLQSGVPSRFSGSGSGT |
| | | | | DFTLTISSLQPEDFATYYCQQAKSFPRTFGQGTKVEI |
| | | | | K |
| 35. | MSLN_5 scFv | artificial | aa | QVQLVESGGGLVKPGGSLRLSCAASGFTFSDYYMT |
| | | | | WIRQAPGKGLEWLSYISSSGSTIYYADSVKGRFTISR |
| | | | | DNAKNSLFLQMNSLRAEDTAVYYCARDRNSHFDY |
| | | | | WGQGTLVTVSSGGGGSGGGGSGGGGSDIQMTQ |
| | | | | SPSSVSASVGDRVTITCRASQGINTWLAWYQQKP |
| | | | | GKAPKLLIYGASGLQSGVPSRFSGSGSGTDFTLTISS |
| | | | | LQPEDFATYYCQQAKSFPRTFGQGTKVEIK |
| 36. | MSLN_5xI2C0 | artificial | aa | QVQLVESGGGLVKPGGSLRLSCAASGFTFSDYYMT |
| | bispecific | | | WIRQAPGKGLEWLSYISSSGSTIYYADSVKGRFTISR |
| | molecule | | | DNAKNSLFLQMNSLRAEDTAVYYCARDRNSHFDY |
| | | | | WGQGTLVTVSSGGGGSGGGGSGGGGSDIQMTQ |
| | | | | SPSSVSASVGDRVTITCRASQGINTWLAWYQQKP |
| | | | | GKAPKLLIYGASGLQSGVPSRFSGSGSGTDFTLTISS |
| | | | | LQPEDFATYYCQQAKSFPRTFGQGTKVEIKSGGGG |
| | | | | SEVQLVESGGGLVQPGGSLKLSCAASGFTFNKYAM |
| | | | | NWVRQAPGKGLEWVARIRSKYNNYATYYADSVKD |
| | | | | RFTISRDDSKNTAYLQMNNLKTEDTAVYYCVRHGN |
| | | | | FGNSYISYWAYWGQGTLVTVSSGGGGSGGGGSG |
| | | | | GGGSQTVVTQEPSLTVSPGGTVTLTCGSSTGAVTS |
| | | | | GNYPNWVQQKPGQAPRGLIGGTKFLAPGTPARFS |
| | | | | GSLLGGKAALTLSGVQPEDEAEYYCVLWYSNRWVF |
| | | | | GGGTKLTVL |
| 37. | MSLN_5xCD3- | artificial | aa | QVQLVESGGGLVKPGGSLRLSCAASGFTFSDYYMT |
| | scFc Bispecific | | | WIRQAPGKGLEWLSYISSSGSTIYYADSVKGRFTISR |
| | HLE molecule | | | DNAKNSLFLQMNSLRAEDTAVYYCARDRNSHFDY |
| | | | | WGQGTLVTVSSGGGGSGGGGSGGGGSDIQMTQ |
| | | | | SPSSVSASVGDRVTITCRASQGINTWLAWYQQKP |
| | | | | GKAPKLLIYGASGLQSGVPSRFSGSGSGTDFTLTISS |
| | | | | LQPEDFATYYCQQAKSFPRTFGQGTKVEIKSGGGG |
| | | | | SEVQLVESGGGLVQPGGSLKLSCAASGFTFNKYAM |
| | | | | NWVRQAPGKGLEWVARIRSKYNNYATYYADSVKD |
| | | | | RFTISRDDSKNTAYLQMNNLKTEDTAVYYCVRHGN |
| | | | | FGNSYISYWAYWGQGTLVTVSSGGGGSGGGGSG |
| | | | | GGGSQTVVTQEPSLTVSPGGTVTLTCGSSTGAVTS |
| | | | | GNYPNWVQQKPGQAPRGLIGGTKFLAPGTPARFS |
| | | | | GSLLGGKAALTLSGVQPEDEAEYYCVLWYSNRWVF |
| | | | | GGGTKLTVLGGGGDKTHTCPPCPAPELLGGPSVFL |
| | | | | FPPKPKDTLMISRTPEVTCVVVDVSHEDPEVKFNW |
| | | | | WLNGKEYKCKVSNKALPAPIEKTISKAKGQPREPQV |
| | | | | YTLPPSREEMTKNQVSLTCLVKGFYPSDIAVEWESN |
| | | | | GQPENNYKTTPPVLDSDGSFFLYSKLTVDKSRWQQ |
| | | | | GNVFSCSVMHEALHNHYTQKSLSLSPGKGGGGSG |
| | | | | GGGSGGGGSGGGGSGGGGSGGGGSDKTHTCPPC |
| | | | | PAPELLGGPSVFLFPPKPKDTLMISRTPEVTCVVVD |
| | | | | VSHEDPEVKFNWYVDGVEVHNAKTKPCEEQYGST |
| | | | | YRCVSVLTVLHQDWLNGKEYKCKVSNKALPAPIEKT |
| | | | | ISKAKGQPREPQVYTLPPSREEMTKNQVSLTCLVKG |
| | | | | FYPSDIAVEWESNGQPENNYKTTPPVLDSDGSFFLY |
| | | | | SKLTVDKSRWQQGNVFSCSVMHEALHNHYTQKSL |
| | | | | SLSPGK |
| 38. | MSLN_5_CCxC | artificial | aa | QVQLVESGGGLVKPGGSLRLSCAASGFTFSDHYMS |
| | D3-scFc | | | WIRQAPGKCLEWFSYISSSGGIIYYADSVKGRFTISR |
| | Bispecific HLE | | | DNAKNSLYLQMNSLRAEDTAVYYCARDVGSHFDY |
| | molecule | | | WGQGTLVTVSSGGGGSGGGGSGGGGSDIQMTQ |
| | | | | SPSSVSASVGDRVTITCRASQDISRWLAWYQQKPG |
| | | | | KAPKLLISAASRLQSGVPSRFSGSGSGTDFTLTISSLQ |
| | | | | PEDFAIYYCQQAKSFPRTFGCGTKVEIKSGGGGSEV |
| | | | | QLVESGGGLVQPGGSLKLSCAASGFTFNKYAMNW |
| | | | | VRQAPGKGLEWVARIRSKYNNYATYYADSVKDRFT |
| | | | | ISRDDSKNTAYLQMNNLKTEDTAVYYCVRHGNFG |
| | | | | NSYISYWAYWGQGTLVTVSSGGGGSGGGGSGGG |
| | | | | GSQTVVTQEPSLTVSPGGTVTLTCGSSTGAVTSGN |
| | | | | YPNWVQQKPGQAPRGLIGGTKFLAPGTPARFSGSL |
| | | | | LGGKAALTLSGVQPEDEAEYYCVLWYSNRWVFGG |
| | | | | GTKLTVLGGGGDKTHTCPPCPAPELLGGPSVFLFPP |
| | | | | KPKDTLMISRTPEVTCVVVDVSHEDPEVKFNWYVD |
| | | | | GVEVHNAKTKPCEEQYGSTYRCVSVLTVLHQDWL |
| | | | | NGKEYKCKVSNKALPAPIEKTISKAKGQPREPQVYT |
| | | | | LPPSREEMTKNQVSLTCLVKGFYPSDIAVEWESNG |
| | | | | QPENNYKTTPPVLDSDGSFFLYSKLTVDKSRWQQG |
| | | | | NVFSCSVMHEALHNHYTQKSLSLSPGKGGGGSGG |
| | | | | GGSGGGGSGGGGSGGGGSGGGGSDKTHTCPPCP |
| | | | | APELLGGPSVFLFPPKPKDTLMISRTPEVTCVVVDV |
| | | | | SHEDPEVKFNWYVDGVEVHNAKTKPCEEQYGSTY |
| | | | | RCVSVLTVLHQDWLNGKEYKCKVSNKALPAPIEKTI |
| | | | | SKAKGQPREPQVYTLPPSREEMTKNQVSLTCLVKG |
| | | | | FYPSDIAVEWESNGQPENNYKTTPPVLDSDGSFFLY |
| | | | | SKLTVDKSRWQQGNVFSCSVMHEALHNHYTQKSL |
| | | | | SLSPGK |
| 39. | CDR-H1 | artificial | aa | SYGMH |
| | CDH19 | | | |
| | 65254.007 | | | |
| 40. | CDR-H2 | artificial | aa | FIWYEGSNKYYAESVKD |
| | CDH19 | | | |
| | 65254.007 | | | |
| 41. | CDR-H3 | artificial | aa | RAGIIGTIGYYYGMDV |
| | CDH19 | | | |
| | 65254.007 | | | |
| 42. | CDR-L1 CDH19 | artificial | aa | SGDRLGEKYTS |
| | 65254.007 | | | |
| 43. | CDR-L2 CDH19 | artificial | aa | QDTKRPS |
| | 65254.007 | | | |
| 44. | CDR-L3 CDH19 | artificial | aa | QAWESSTVV |
| | 65254.007 | | | |
| 45. | VH CDH19 | artificial | aa | QVQLVESGGGVVQPGGSLRLSCAASGFTFSSYGM |
| | 65254.007 | | | HWVRQAPGKGLEWVAFIWYEGSNKYYAESVKDRF |
| | | | | TISRDNSKNTLYLQMNSLRAEDTAVYYCARRAGIIG |
| | | | | TIGYYYGMDVWGQGTTVTVSS |
| 46. | VL CDH19 | artificial | aa | SYELTQPPSVSVSPGQTASITCSGDRLGEKYTSWYQ |
| | 65254.007 | | | QRPGQSPLLVIYQDTKRPSGIPERFSGSNSGNTATL |
| | | | | TISGTQAMDEADYYCQAWESSTVVFGGGTKLTVLS |
| 47. | VH-VL CDH19 | artificial | aa | QVQLVESGGGVVQPGGSLRLSCAASGFTFSSYGM |
| | 65254.007 | | | HWVRQAPGKGLEWVAFIWYEGSNKYYAESVKDRF |
| | | | | TISRDNSKNTLYLQMNSLRAEDTAVYYCARRAGIIG |
| | | | | TIGYYYGMDVWGQGTTVTVSSGGGGSGGGGSGG |
| | | | | GGSSYELTQPPSVSVSPGQTASITCSGDRLGEKYTS |
| | | | | WYQQRPGQSPLLVIYQDTKRPSGIPERFSGSNSGN |
| | | | | TATLTISGTQAMDEADYYCQAWESSTVVFGGGTKL |
| | | | | TVLS |
| 48. | CDH19 | artificial | aa | QVQLVESGGGVVQPGGSLRLSCAASGFTFSSYGM |
| | 65254.007 x | | | HWVRQAPGKGLEWVAFIWYEGSNKYYAESVKDRF |
| | I2C | | | TISRDNSKNTLYLQMNSLRAEDTAVYYCARRAGIIG |
| | | | | TIGYYYGMDVWGQGTTVTVSSGGGGSGGGGSGG |
| | | | | GGSSYELTQPPSVSVSPGQTASITCSGDRLGEKYTS |
| | | | | WYQQRPGQSPLLVIYQDTKRPSGIPERFSGSNSGN |
| | | | | TATLTISGTQAMDEADYYCQAWESSTVVFGGGTKL |
| | | | | TVLSGGGGSEVQLVESGGGLVQPGGSLKLSCAASG |
| | | | | FTFNKYAMNWVRQAPGKGLEWVARIRSKYNNYA |
| | | | | TYYADSVKDRFTISRDDSKNTAYLQMNNLKTEDTA |
| | | | | VYYCVRHGNFGNSYISYWAYWGQGTLVTVSSGGG |
| | | | | GSGGGGSGGGGSQTVVTQEPSLTVSPGGTVTLTC |
| | | | | GSSTGAVTSGNYPNWVQQKPGQAPRGLIGGTKFL |
| | | | | APGTPARFSGSLLGGKAALTLSGVQPEDEAEYYCVL |
| | | | | WYSNRWVFGGGTKLTVLHHHHHH |
| 49. | CDH19 | artificial | aa | QVQLVESGGGVVQPGGSLRLSCAASGFTFSSYGMH |
| | 65254.007 x | | | WVRQAPGKGLEWVAFIWYEGSNKYYAESVKDRFTIS |
| | I2C-scFc | | | RDNSKNTLYLQMNSLRAEDTAVYYCARRAGIIGTIGY |
| | Bispecific HLE | | | YELTQPPSVSVSPGQTASITCSGDRLGEKYTSWYQQR |
| | molecule | | | PGQSPLLVIYQDTKRPSGIPERFSGSNSGNTATLTISG |
| | | | | TQAMDEADYYCQAWESSTVVFGGGTKLTVLSGGGG |
| | | | | SEVQLVESGGGLVQPGGSLKLSCAASGFTFNKYAMN |
| | | | | WVRQAPGKGLEWVARIRSKYNNYATYYADSVKDRF |
| | | | | TISRDDSKNTAYLQMNNLKTEDTAVYYCVRHGNFGN |
| | | | | SYISYWAYWGQGTLVTVSSGGGGSGGGGSGGGGS |
| | | | | QTVVTQEPSLTVSPGGTVTLTCGSSTGAVTSGNYPN |
| | | | | WVQQKPGQAPRGLIGGTKFLAPGTPARFSGSLLGGK |
| | | | | AALTLSGVQPEDEAEYYCVLWYSNRWVFGGGTKLTV |
| | | | | LGGGGDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGKGGGGSGGGGSGGGGSGGGG |
| | | | | SGGGGSGGGGSDKTHTCPPCPAPELLGGPSVFLFPP |
| | | | | KPKDTLMISRTPEVTCVVVDVSHEDPEVKFNWYVDG |
| | | | | VEVHNAKTKPCEEQYGSTYRCVSVLTVLHQDWLNG |
| | | | | KEYKCKVSNKALPAPIEKTISKAKGQPREPQVYTLPPS |
| | | | | REEMTKNQVSLTCLVKGFYPSDIAVEWESNGQPENN |
| | | | | YKTTPPVLDSDGSFFLYSKLTVDKSRWQQGNVFSCSV |
| | | | | MHEALHNHYTQKSLSLSPGK |
| 50. | CDH19 | artificial | aa | QVQLVESGGGVVQPGGSLRLSCAASGFTFSSYGMH |
| | 65254.007 x | | | WVRQAPGKGLEWVAFIWYEGSNKYYAESVKDRFTIS |
| | I2C- | | | RDNSKNTLYLQMNSLRAEDTAVYYCARRAGIIGTIGY |
| | scFc_delGK | | | YYGMDVWGQGTTVTVSSGGGGSGGGGSGGGGSS |
| | Bispecific HLE | | | YELTQPPSVSVSPGQTASITCSGDRLGEKYTSWYQQR |
| | molecule | | | PGQSPLLVIYQDTKRPSGIPERFSGSNSGNTATLTISG |
| | | | | TQAMDEADYYCQAWESSTVVFGGGTKLTVLSGGGG |
| | | | | SEVQLVESGGGLVQPGGSLKLSCAASGFTFNKYAMN |
| | | | | WVRQAPGKGLEWVARIRSKYNNYATYYADSVKDRF |
| | | | | TISRDDSKNTAYLQMNNLKTEDTAVYYCVRHGNFGN |
| | | | | SYISYWAYWGQGTLVTVSSGGGGSGGGGSGGGGS |
| | | | | QTVVTQEPSLTVSPGGTVTLTCGSSTGAVTSGNYPN |
| | | | | WVQQKPGQAPRGLIGGTKFLAPGTPARFSGSLLGGK |
| | | | | AALTLSGVQPEDEAEYYCVLWYSNRWVFGGGTKLTV |
| | | | | LGGGGDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGGGGSGGGGSGGGGSGGGGSG |
| | | | | GGGSGGGGSDKTHTCPPCPAPELLGGPSVFLFPPKP |
| | | | | KDTLMISRTPEVTCVVVDVSHEDPEVKFNWYVDGVE |
| | | | | VHNAKTKPCEEQYGSTYRCVSVLTVLHQDWLNGKEY |
| | | | | KCKVSNKALPAPIEKTISKAKGQPREPQVYTLPPSREE |
| | | | | MTKNQVSLTCLVKGFYPSDIAVEWESNGQPENNYKT |
| | | | | TPPVLDSDGSFFLYSKLTVDKSRWQQGNVFSCSVMH |
| | | | | EALHNHYTQKSLSLSPGK |
| 51. | CDH19 | artificial | aa | QVQLVESGGGVVQPGGSLRLSCAASGFTFSSYGMH |
| | 65254.007_CC | | | WVRQAPGKCLEWVAFIWYEGSNKYYAESVKDRFTIS |
| | x I2C-scFc VH | | | RDNSKNTLYLQMNSLRAEDTAVYYCARRAGIIGTIGY |
| | | | | YYGMDVWGQGTTVTVSS |
| 52. | CDH19 | artificial | aa | SYELTQPPSVSVSPGQTASITCSGDRLGEKYTSWYQQ |
| | 65254.007_CC | | | RPGQSPLLVIYQDTKRPSGIPERFSGSNSGNTATLTIS |
| | x I2C-scFc VL | | | GTQAMDEADYYCQAWESSTVVFGCGTKLTVL |
| 53. | CDH19 | artificial | aa | QVQLVESGGGVVQPGGSLRLSCAASGFTFSSYGMH |
| | 65254.007_CC | | | WVRQAPGKCLEWVAFIWYEGSNKYYAESVKDRFTIS |
| | x I2C-scFc scFv | | | RDNSKNTLYLQMNSLRAEDTAVYYCARRAGIIGTIGY |
| | | | | YYGMDVWGQGTTVTVSSGGGGSGGGGSGGGGSS |
| | | | | YELTQPPSVSVSPGQTASITCSGDRLGEKYTSWYQQR |
| | | | | PGQSPLLVIYQDTKRPSGIPERFSGSNSGNTATLTISG |
| | | | | TQAMDEADYYCQAWESSTVVFGCGTKLTVL |
| 54. | CDH19 | artificial | aa | QVQLVESGGGVVQPGGSLRLSCAASGFTFSSYGMH |
| | 65254.007_CC | | | WVRQAPGKCLEWVAFIWYEGSNKYYAESVKDRFTIS |
| | x I2C-scFc | | | RDNSKNTLYLQMNSLRAEDTAVYYCARRAGIIGTIGY |
| | Bispecific | | | YYGMDVWGQGTTVTVSSGGGGSGGGGSGGGGSS |
| | molecule | | | YELTQPPSVSVSPGQTASITCSGDRLGEKYTSWYQQR |
| | | | | PGQSPLLVIYQDTKRPSGIPERFSGSNSGNTATLTISG |
| | | | | TQAMDEADYYCQAWESSTVVFGCGTKLTVLSGGGG |
| | | | | SEVQLVESGGGLVQPGGSLKLSCAASGFTFNKYAMN |
| | | | | WVRQAPGKGLEWVARIRSKYNNYATYYADSVKDRF |
| | | | | TISRDDSKNTAYLQMNNLKTEDTAVYYCVRHGNFGN |
| | | | | SYISYWAYWGQGTLVTVSSGGGGSGGGGSGGGGS |
| | | | | QTVVTQEPSLTVSPGGTVTLTCGSSTGAVTSGNYPN |
| | | | | WVQQKPGQAPRGLIGGTKFLAPGTPARFSGSLLGGK |
| | | | | AALTLSGVQPEDEAEYYCVLWYSNRWVFGGGTKLTV |
| | | | | L |
| 55. | CDH19 | artificial | aa | QVQLVESGGGVVQPGGSLRLSCAASGFTFSSYGMH |
| | 65254.007_CC | | | WVRQAPGKCLEWVAFIWYEGSNKYYAESVKDRFTIS |
| | x I2C-scFc | | | RDNSKNTLYLQMNSLRAEDTAVYYCARRAGIIGTIGY |
| | Bispecific HLE | | | YYGMDVWGQGTTVTVSSGGGGSGGGGSGGGGSS |
| | molecule | | | YELTQPPSVSVSPGQTASITCSGDRLGEKYTSWYQQR |
| | | | | PGQSPLLVIYQDTKRPSGIPERFSGSNSGNTATLTISG |
| | | | | TQAMDEADYYCQAWESSTVVFGCGTKLTVLSGGGG |
| | | | | SEVQLVESGGGLVQPGGSLKLSCAASGFTFNKYAMN |
| | | | | WVRQAPGKGLEWVARIRSKYNNYATYYADSVKDRF |
| | | | | TISRDDSKNTAYLQMNNLKTEDTAVYYCVRHGNFGN |
| | | | | SYISYWAYWGQGTLVTVSSGGGGSGGGGSGGGGS |
| | | | | QTVVTQEPSLTVSPGGTVTLTCGSSTGAVTSGNYPN |
| | | | | WVQQKPGQAPRGLIGGTKFLAPGTPARFSGSLLGGK |
| | | | | AALTLSGVQPEDEAEYYCVLWYSNRWVFGGGTKLTV |
| | | | | LGGGGDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGKGGGGSGGGGSGGGGSGGGG |
| | | | | SGGGGSGGGGSDKTHTCPPCPAPELLGGPSVFLFPP |
| | | | | KPKDTLMISRTPEVTCVVVDVSHEDPEVKFNWYVDG |
| | | | | VEVHNAKTKPCEEQYGSTYRCVSVLTVLHQDWLNG |
| | | | | KEYKCKVSNKALPAPIEKTISKAKGQPREPQVYTLPPS |
| | | | | REEMTKNQVSLTCLVKGFYPSDIAVEWESNGQPENN |
| | | | | YKTTPPVLDSDGSFFLYSKLTVDKSRWQQGNVFSCSV |
| | | | | MHEALHNHYTQKSLSLSPGK |
| 56. | CDH19 | artificial | aa | QVQLVESGGGVVQPGGSLRLSCAASGFTFSSYGMH |
| | 65254.007_CC | | | WVRQAPGKCLEWVAFIWYEGSNKYYAESVKDRFTIS |
| | x I2C- | | | RDNSKNTLYLQMNSLRAEDTAVYYCARRAGIIGTIGY |
| | scFc_delGK | | | YYGMDVWGQGTTVTVSSGGGGSGGGGSGGGGSS |
| | Bispecific HLE | | | YELTQPPSVSVSPGQTASITCSGDRLGEKYTSWYQQR |
| | molecule | | | PGQSPLLVIYQDTKRPSGIPERFSGSNSGNTATLTISG |
| | | | | TQAMDEADYYCQAWESSTVVFGCGTKLTVLSGGGG |
| | | | | SEVQLVESGGGLVQPGGSLKLSCAASGFTFNKYAMN |
| | | | | WVRQAPGKGLEWVARIRSKYNNYATYYADSVKDRF |
| | | | | TISRDDSKNTAYLQMNNLKTEDTAVYYCVRHGNFGN |
| | | | | SYISYWAYWGQGTLVTVSSGGGGSGGGGSGGGGS |
| | | | | QTVVTQEPSLTVSPGGTVTLTCGSSTGAVTSGNYPN |
| | | | | WVQQKPGQAPRGLIGGTKFLAPGTPARFSGSLLGGK |
| | | | | AALTLSGVQPEDEAEYYCVLWYSNRWVFGGGTKLTV |
| | | | | LGGGGDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGGGGSGGGGSGGGGSGGGGSG |
| | | | | GGGSGGGGSDKTHTCPPCPAPELLGGPSVFLFPPKP |
| | | | | KDTLMISRTPEVTCVVVDVSHEDPEVKFNWYVDGVE |
| | | | | VHNAKTKPCEEQYGSTYRCVSVLTVLHQDWLNGKEY |
| | | | | KCKVSNKALPAPIEKTISKAKGQPREPQVYTLPPSREE |
| | | | | MTKNQVSLTCLVKGFYPSDIAVEWESNGQPENNYKT |
| | | | | TPPVLDSDGSFFLYSKLTVDKSRWQQGNVFSCSVMH |
| | | | | EALHNHYTQKSLSLSPGK |
| 57. | FLT3_7 | artificial | aa | NARMGVS |
| | A8xCD3-scFc | | | |
| | VH CDR1 | | | |
| 58. | FLT3_7 | artificial | aa | HIFSNDEKSYSTSLKN |
| | A8xCD3-scFc | | | |
| | VH CDR2 | | | |
| 59. | FLT3_7 | artificial | aa | IVGYGSGWYGFFDY |
| | A8xCD3-scFc | | | |
| | VH CDR3 | | | |
| 60. | FLT3_7 | artificial | aa | RASQGIRNDLG |
| | A8xCD3-scFc VL | | | |
| | CDR1 | | | |
| 61. | FLT3_7 | artificial | aa | AASTLOS |
| | A8xCD3-scFc VL | | | |
| | CDR2 | | | |
| 62. | FLT3_7 | artificial | aa | LQHNSYPLT |
| | A8xCD3-scFc VL | | | |
| | CDR3 | | | |
| 63. | FLT3_7 | artificial | aa | QVTLKESGPTLVKPTETLTLTCTLSGFSLNNARMGVS |
| | A8xCD3-scFc | | | WIRQPPGKCLEWLAHIFSNDEKSYSTSLKNRLTISKDS |
| | VH | | | SKTQVVLTMTNVDPVDTATYYCARIVGYGSGWYGFF |
| | | | | DYWGQGTLVTVSS |
| 64. | FLT3_ A8-scFc | artificial | aa | DIQMTQSPSSLSASVGDRVTITCRASQGIRNDLGWY |
| | VL | | | QQKPGKAPKRLIYAASTLQSGVPSRFSGSGSGTEFTLT |
| | | | | ISSLQPEDFATYYCLQHNSYPLTFGCGTKVEIK |
| 65. | FLT3_7 | artificial | aa | QVTLKESGPTLVKPTETLTLTCTLSGFSLNNARMGVS |
| | A8xCD3-scFv | | | WIRQPPGKCLEWLAHIFSNDEKSYSTSLKNRLTISKDS |
| | | | | SKTQVVLTMTNVDPVDTATYYCARIVGYGSGWYGFF |
| | | | | DYWGQGTLVTVSSGGGGSGGGGSGGGGSDIQMT |
| | | | | QSPSSLSASVGDRVTITCRASQGIRNDLGWYQQKPG |
| | | | | KAPKRLIYAASTLQSGVPSRFSGSGSGTEFTLTISSLQP |
| | | | | EDFATYYCLQHNSYPLTFGCGTKVEIK |
| 66. | FLT3_7 A8xCD3 | artificial | aa | QVTLKESGPTLVKPTETLTLTCTLSGFSLNNARMGVS |
| | Bispecific | | | WIRQPPGKCLEWLAHIFSNDEKSYSTSLKNRLTISKDS |
| | molecule | | | SKTQVVLTMTNVDPVDTATYYCARIVGYGSGWYGFF |
| | | | | DYWGQGTLVTVSSGGGGSGGGGSGGGGSDIQMT |
| | | | | QSPSSLSASVGDRVTITCRASQGIRNDLGWYQQKPG |
| | | | | KAPKRLIYAASTLQSGVPSRFSGSGSGTEFTLTISSLQP |
| | | | | EDFATYYCLQHNSYPLTFGCGTKVEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVL |
| 67. | FLT3_7 | artificial | aa | QVTLKESGPTLVKPTETLTLTCTLSGFSLNNARMGVS |
| | A8xCD3-scFc | | | WIRQPPGKCLEWLAHIFSNDEKSYSTSLKNRLTISKDS |
| | Bispecific HLE | | | SKTQVVLTMTNVDPVDTATYYCARIVGYGSGWYGFF |
| | molecule | | | DYWGQGTLVTVSSGGGGSGGGGSGGGGSDIQMT |
| | | | | QSPSSLSASVGDRVTITCRASQGIRNDLGWYQQKPG |
| | | | | KAPKRLIYAASTLQSGVPSRFSGSGSGTEFTLTISSLQP |
| | | | | EDFATYYCLQHNSYPLTFGCGTKVEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVLGGG |
| | | | | GDKTHTCPPCPAPELLGGPSVFLFPPKPKDTLMISRTP |
| | | | | EVTCVVVDVSHEDPEVKFNWYVDGVEVHNAKTKPC |
| | | | | EEQYGSTYRCVSVLTVLHQDWLNGKEYKCKVSNKAL |
| | | | | PAPIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGKGGGGSGGGGSGGGGSGGGGSGGGG |
| | | | | SGGGGSDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGK |
| 68 | VH CDR1 | artificial | aa | SYYWS |
| | DLL3_1_CC_del | | | |
| | GK | | | |
| 69 | VH CDR2 | artificial | aa | YVYYSGTTNYNPSLKS |
| | DLL3_1_CC_del | | | |
| | GK | | | |
| 70 | VH CDR3 | artificial | aa | IAVTGFYFDY |
| | DLL3_1_CC_del | | | |
| | GK | | | |
| 71 | VLCDR1 | artificial | aa | RASQRVNNNYLA |
| | DLL3_1_CC_del | | | |
| | GK | | | |
| 72 | VLCDR2 | artificial | aa | GASSRAT |
| | DLL3_1_CC_del | | | |
| | GK | | | |
| 73 | VLCDR3 | artificial | aa | QQYDRSPLT |
| | DLL3_1_CC_del | | | |
| | GK | | | |
| 74. | VH | artificial | aa | QVQLQESGPGLVKPSETLSLTCTVSGGSISSYYWSWI |
| | DLL3_1_CC_del | | | RQPPGKCLEWIGYVYYSGTTNYNPSLKSRVTISVDTSK |
| | GK | | | NQFSLKLSSVTAADTAVYYCASIAVTGFYFDYWGQG |
| | | | | TLVTVSS |
| 75. | VL | artificial | aa | EIVLTQSPGTLSLSPGERVTLSCRASQRVNNNYLAWY |
| | DLL3_1_CC_del | | | QQRPGQAPRLLIYGASSRATGIPDRFSGSGSGTDFTL |
| | GK | | | TISRLEPEDFAVYYCQQYDRSPLTFGCGTKLEIK |
| 76. | DLL3_1_CC_del | artificial | aa | QVQLQESGPGLVKPSETLSLTCTVSGGSISSYYWSWI |
| | GK | | | RQPPGKCLEWIGYVYYSGTTNYNPSLKSRVTISVDTSK |
| | | | | NQFSLKLSSVTAADTAVYYCASIAVTGFYFDYWGQG |
| | | | | TLVTVSSGGGGSGGGGSGGGGSEIVLTQSPGTLSLSP |
| | | | | GERVTLSCRASQRVNNNYLAWYQQRPGQAPRLLIY |
| | | | | GASSRATGIPDRFSGSGSGTDFTLTISRLEPEDFAVYY |
| | | | | CQQYDRSPLTFGCGTKLEIK |
| 77. | DLL3_1_CCxCD | artificial | aa | QVQLQESGPGLVKPSETLSLTCTVSGGSISSYYWSWI |
| | 3_delGK | | | RQPPGKCLEWIGYVYYSGTTNYNPSLKSRVTISVDTSK |
| | Bispecific | | | NQFSLKLSSVTAADTAVYYCASIAVTGFYFDYWGQG |
| | molecule | | | TLVTVSSGGGGSGGGGSGGGGSEIVLTQSPGTLSLSP |
| | | | | GERVTLSCRASQRVNNNYLAWYQQRPGQAPRLLIY |
| | | | | GASSRATGIPDRFSGSGSGTDFTLTISRLEPEDFAVYY |
| | | | | CQQYDRSPLTFGCGTKLEIKSGGGGSEVQLVESGGGL |
| | | | | VQPGGSLKLSCAASGFTFNKYAMNWVRQAPGKGLE |
| | | | | WVARIRSKYNNYATYYADSVKDRFTISRDDSKNTAYL |
| | | | | QMNNLKTEDTAVYYCVRHGNFGNSYISYWAYWGQ |
| | | | | GTLVTVSSGGGGSGGGGSGGGGSQTVVTQEPSLTV |
| | | | | SPGGTVTLTCGSSTGAVTSGNYPNWVQQKPGQAPR |
| | | | | GLIGGTKFLAPGTPARFSGSLLGGKAALTLSGVQPED |
| | | | | EAEYYCVLWYSNRWVFGGGTKLTVL |
| 78. | DLL3_1_CCxCD | artificial | aa | QVQLQESGPGLVKPSETLSLTCTVSGGSISSYYWSWI |
| | 3-scFc_delGK | | | RQPPGKCLEWIGYVYYSGTTNYNPSLKSRVTISVDTSK |
| | Bispecific HLE | | | NQFSLKLSSVTAADTAVYYCASIAVTGFYFDYWGQG |
| | molecule | | | TLVTVSSGGGGSGGGGSGGGGSEIVLTQSPGTLSLSP |
| | | | | GERVTLSCRASQRVNNNYLAWYQQRPGQAPRLLIY |
| | | | | GASSRATGIPDRFSGSGSGTDFTLTISRLEPEDFAVYY |
| | | | | CQQYDRSPLTFGCGTKLEIKSGGGGSEVQLVESGGGL |
| | | | | VQPGGSLKLSCAASGFTFNKYAMNWVRQAPGKGLE |
| | | | | WVARIRSKYNNYATYYADSVKDRFTISRDDSKNTAYL |
| | | | | QMNNLKTEDTAVYYCVRHGNFGNSYISYWAYWGQ |
| | | | | GTLVTVSSGGGGSGGGGSGGGGSQTVVTQEPSLTV |
| | | | | SPGGTVTLTCGSSTGAVTSGNYPNWVQQKPGQAPR |
| | | | | GLIGGTKFLAPGTPARFSGSLLGGKAALTLSGVQPED |
| | | | | EAEYYCVLWYSNRWVFGGGTKLTVLGGGGDKTHTC |
| | | | | PPCPAPELLGGPSVFLFPPKPKDTLMISRTPEVTCVVV |
| | | | | DVSHEDPEVKFNWYVDGVEVHNAKTKPCEEQYGST |
| | | | | YRCVSVLTVLHQDWLNGKEYKCKVSNKALPAPIEKTI |
| | | | | SKAKGQPREPQVYTLPPSREEMTKNQVSLTCLVKGF |
| | | | | YPSDIAVEWESNGQPENNYKTTPPVLDSDGSFFLYSK |
| | | | | LTVDKSRWQQGNVFSCSVMHEALHNHYTQKSLSLS |
| | | | | PGGGGSGGGGSGGGGSGGGGSGGGGSGGGGSDK |
| | | | | THTCPPCPAPELLGGPSVFLFPPKPKDTLMISRTPEVT |
| | | | | CVVVDVSHEDPEVKFNWYVDGVEVHNAKTKPCEEQ |
| | | | | YGSTYRCVSVLTVLHQDWLNGKEYKCKVSNKALPAPI |
| | | | | EKTISKAKGQPREPQVYTLPPSREEMTKNQVSLTCLV |
| | | | | KGFYPSDIAVEWESNGQPENNYKTTPPVLDSDGSFFL |
| | | | | YSKLTVDKSRWQQGNVFSCSVMHEALHNHYTQKSL |
| | | | | SLSPGK |
| 79. | VH CDR1 CD19 | artificial | aa | SYGMH |
| | 97-G1RE-C2 | | | |
| 80. | VH CDR2 CD19 | artificial | aa | VISYEGSNKYYAESVKG |
| | 97-G1RE-C2 | | | |
| 81. | VH CDR3 CD19 | artificial | aa | DRGTIFGNYGLEV |
| | 97-G1RE-C2 | | | |
| 82. | VH CD19 97- | artificial | aa | QVQLVESGGGVVQPGRSLRLSCAASGFTFSSYGMH |
| | G1RE-C2 CC | | | WVRQAPGKCLEWVAVISYEGSNKYYAESVKGRFTIS |
| | | | | RDNSKNTLYLQMNSLRDEDTAVYYCARDRGTIFGNY |
| | | | | GLEVWGQGTTVTVSS |
| 83. | VL CDR1 CD19 | artificial | aa | RSSQSLLHKNAFNYLD |
| | 97-G1RE-C2 | | | |
| 84. | VL CDR2 CD19 | artificial | aa | LGSNRAS |
| | 97-G1RE-C2 | | | |
| 85. | VL CDR3 CD19 | artificial | aa | MQALQTPFT |
| | 97-G1RE-C2 | | | |
| 86. | VL CD19 97- | artificial | aa | DIVMTQSPLSLPVISGEPASISCRSSQSLLHKNAFNYL |
| | G1RE-C2 CC | | | DWYLQKPGQSPQLLIYLGSNRASGVPDRFSGSGSGT |
| | | | | DFTLKISRVEAEDVGVYYCMQALQTPFTFGCGTKVDI |
| | | | | K |
| 87. | CD19 97-G1RE- | artificial | aa | MDMRVPAQLLGLLLLWLRGARCDIVMTQSPLSLPVI |
| | C2 CCxI2C0 | | | SGEPASISCRSSQSLLHKNAFNYLDWYLQKPGQSPQL |
| | | | | LIYLGSNRASGVPDRFSGSGSGTDFTLKISRVEAEDVG |
| | | | | VYYCMQALQTPFTFGCGTKVDIKGGGGSGGGGSGG |
| | | | | GGSQVQLVESGGGVVQPGRSLRLSCAASGFTFSSYG |
| | | | | MHWVRQAPGKCLEWVAVISYEGSNKYYAESVKGRF |
| | | | | TISRDNSKNTLYLQMNSLRDEDTAVYYCARDRGTIFG |
| | | | | NYGLEVWGQGTTVTVSSGGGGSEVQLVESGGGLVQ |
| | | | | PGGSLKLSCAASGFTFNKYAMNWVRQAPGKGLEW |
| | | | | VARIRSKYNNYATYYADSVKDRFTISRDDSKNTAYLQ |
| | | | | MNNLKTEDTAVYYCVRHGNFGNSYISYWAYWGQG |
| | | | | TLVTVSSGGGGSGGGGSGGGGSQTVVTQEPSLTVSP |
| | | | | GGTVTLTCGSSTGAVTSGNYPNWVQQKPGQAPRGL |
| | | | | IGGTKFLAPGTPARFSGSLLGGKAALTLSGVQPEDEA |
| | | | | EYYCVLWYSNRWVFGGGTKLTVL |
| 88. | CD19 97-G1RE- | artificial | aa | MDMRVPAQLLGLLLLWLRGARCDIVMTQSPLSLPVI |
| | C2 CCxI2C0- | | | SGEPASISCRSSQSLLHKNAFNYLDWYLQKPGQSPQL |
| | scFc | | | LIYLGSNRASGVPDRFSGSGSGTDFTLKISRVEAEDVG |
| | | | | VYYCMQALQTPFTFGCGTKVDIKGGGGSGGGGSGG |
| | | | | GGSQVQLVESGGGVVQPGRSLRLSCAASGFTFSSYG |
| | | | | MHWVRQAPGKCLEWVAVISYEGSNKYYAESVKGRF |
| | | | | TISRDNSKNTLYLQMNSLRDEDTAVYYCARDRGTIFG |
| | | | | NYGLEVWGQGTTVTVSSGGGGSEVQLVESGGGLVQ |
| | | | | PGGSLKLSCAASGFTFNKYAMNWVRQAPGKGLEW |
| | | | | VARIRSKYNNYATYYADSVKDRFTISRDDSKNTAYLQ |
| | | | | MNNLKTEDTAVYYCVRHGNFGNSYISYWAYWGQG |
| | | | | TLVTVSSGGGGSGGGGSGGGGSQTVVTQEPSLTVSP |
| | | | | GGTVTLTCGSSTGAVTSGNYPNWVQQKPGQAPRGL |
| | | | | IGGTKFLAPGTPARFSGSLLGGKAALTLSGVQPEDEA |
| | | | | EYYCVLWYSNRWVFGGGTKLTVLGGGGDKTHTCPP |
| | | | | CPAPELLGGPSVFLFPPKPKDTLMISRTPEVTCVVVDV |
| | | | | SHEDPEVKFNWYVDGVEVHNAKTKPCEEQYGSTYR |
| | | | | CVSVLTVLHQDWLNGKEYKCKVSNKALPAPIEKTISK |
| | | | | AKGQPREPQVYTLPPSREEMTKNQVSLTCLVKGFYP |
| | | | | SDIAVEWESNGQPENNYKTTPPVLDSDGSFFLYSKLT |
| | | | | VDKSRWQQGNVFSCSVMHEALHNHYTQKSLSLSPG |
| | | | | KGGGGSGGGGSGGGGSGGGGSGGGGSGGGGSDK |
| | | | | THTCPPCPAPELLGGPSVFLFPPKPKDTLMISRTPEVT |
| | | | | CVVVDVSHEDPEVKFNWYVDGVEVHNAKTKPCEEQ |
| | | | | YGSTYRCVSVLTVLHQDWLNGKEYKCKVSNKALPAPI |
| | | | | EKTISKAKGQPREPQVYTLPPSREEMTKNQVSLTCLV |
| | | | | KGFYPSDIAVEWESNGQPENNYKTTPPVLDSDGSFFL |
| | | | | YSKLTVDKSRWQQGNVFSCSVMHEALHNHYTQKSL |
| | | | | SLSPGK |
| 89. | VH CDR1 CDH3 | artificial | aa | SYPIN |
| | G8A 6-B12 | | | |
| 90. | VH CDR2 CDH3 | artificial | aa | VIWTGGGTNYASSVKG |
| | G8A 6-B12 | | | |
| 91. | VH CDR3 CDH3 | artificial | aa | SRGVYDFDGRGAMDY |
| | G8A 6-B12 | | | |
| 92. | VL CDR1 CDH3 | artificial | aa | KSSQSLLYSSNQKNYFA |
| | G8A 6-B12 | | | |
| 93. | VL CDR2 CDH3 | artificial | aa | WASTRES |
| | G8A 6-B12 | | | |
| 94. | VL CDR3 CDH3 | artificial | aa | QQYYSYPYT |
| | G8A 6-B12 | | | |
| 95. | VH CDH3 G8A | artificial | aa | EVQLLESGGGLVQPGGSLRLSCAASGFSFSSYPINWV |
| | 6-B12 | | | RQAPGKGLEWVGVIWTGGGTNYASSVKGRFTISRD |
| | | | | NSKNTVYLQMNSLRAEDTAVYYCAKSRGVYDFDGR |
| | | | | GAMDYWGQGTLVTVSS |
| 96. | VL CDH3 G8A | artificial | aa | DIVMTQSPDSLAVSLGERATINCKSSQSLLYSSNQKN |
| | 6-B12 | | | YFAWYQQKPGQPPKLLIYWASTRESGVPDRFSGSGS |
| | | | | GTDFTLTISSLQAEDVAVYYCQQYYSYPYTFGQGTKL |
| | | | | EIK |
| 97. | CDH3 G8A 6- | artificial | aa | EVQLLESGGGLVQPGGSLRLSCAASGFSFSSYPINWV |
| | B12 scFv | | | RQAPGKGLEWVGVIWTGGGTNYASSVKGRFTISRD |
| | | | | NSKNTVYLQMNSLRAEDTAVYYCAKSRGVYDFDGR |
| | | | | GAMDYWGQGTLVTVSSGGGGSGGGGSGGGGSDI |
| | | | | VMTQSPDSLAVSLGERATINCKSSQSLLYSSNQKNYF |
| | | | | AWYQQKPGQPPKLLIYWASTRESGVPDRFSGSGSGT |
| | | | | DFTLTISSLQAEDVAVYYCQQYYSYPYTFGQGTKLEIK |
| 98. | CDH3 G8A 6- | artificial | aa | EVQLLESGGGLVQPGGSLRLSCAASGFSFSSYPINWV |
| | B12 x I2C0 | | | RQAPGKGLEWVGVIWTGGGTNYASSVKGRFTISRD |
| | bispecific | | | NSKNTVYLQMNSLRAEDTAVYYCAKSRGVYDFDGR |
| | molecule | | | GAMDYWGQGTLVTVSSGGGGSGGGGSGGGGSDI |
| | | | | VMTQSPDSLAVSLGERATINCKSSQSLLYSSNQKNYF |
| | | | | AWYQQKPGQPPKLLIYWASTRESGVPDRFSGSGSGT |
| | | | | DFTLTISSLQAEDVAVYYCQQYYSYPYTFGQGTKLEIK |
| | | | | SGGGGSEVQLVESGGGLVQPGGSLKLSCAASGFTFN |
| | | | | KYAMNWVRQAPGKGLEWVARIRSKYNNYATYYAD |
| | | | | SVKDRFTISRDDSKNTAYLQMNNLKTEDTAVYYCVR |
| | | | | HGNFGNSYISYWAYWGQGTLVTVSSGGGGSGGGG |
| | | | | SGGGGSQTVVTQEPSLTVSPGGTVTLTCGSSTGAVTS |
| | | | | GNYPNWVQQKPGQAPRGLIGGTKFLAPGTPARFSG |
| | | | | SLLGGKAALTLSGVQPEDEAEYYCVLWYSNRWVFGG |
| | | | | GTKLTVL |
| 99. | CDH3 G8A 6- | artificial | aa | EVQLLESGGGLVQPGGSLRLSCAASGFSFSSYPINWV |
| | B12 x I2C0 | | | RQAPGKGLEWVGVIWTGGGTNYASSVKGRFTISRD |
| | bispecific | | | NSKNTVYLQMNSLRAEDTAVYYCAKSRGVYDFDGR |
| | molecule HLE | | | GAMDYWGQGTLVTVSSGGGGSGGGGSGGGGSDI |
| | | | | VMTQSPDSLAVSLGERATINCKSSQSLLYSSNQKNYF |
| | | | | AWYQQKPGQPPKLLIYWASTRESGVPDRFSGSGSGT |
| | | | | DFTLTISSLQAEDVAVYYCQQYYSYPYTFGQGTKLEIK |
| | | | | SGGGGSEVQLVESGGGLVQPGGSLKLSCAASGFTFN |
| | | | | KYAMNWVRQAPGKGLEWVARIRSKYNNYATYYAD |
| | | | | SVKDRFTISRDDSKNTAYLQMNNLKTEDTAVYYCVR |
| | | | | HGNFGNSYISYWAYWGQGTLVTVSSGGGGSGGGG |
| | | | | SGGGGSQTVVTQEPSLTVSPGGTVTLTCGSSTGAVTS |
| | | | | GNYPNWVQQKPGQAPRGLIGGTKFLAPGTPARFSG |
| | | | | SLLGGKAALTLSGVQPEDEAEYYCVLWYSNRWVFGG |
| | | | | GTKLTVLGGGGDKTHTCPPCPAPELLGGPSVFLFPPK |
| | | | | PKDTLMISRTPEVTCVVVDVSHEDPEVKFNWYVDGV |
| | | | | EVHNAKTKPCEEQYGSTYRCVSVLTVLHQDWLNGKE |
| | | | | YKCKVSNKALPAPIEKTISKAKGQPREPQVYTLPPSRE |
| | | | | EMTKNQVSLTCLVKGFYPSDIAVEWESNGQPENNYK |
| | | | | TTPPVLDSDGSFFLYSKLTVDKSRWQQGNVFSCSVM |
| | | | | HEALHNHYTQKSLSLSPGKGGGGSGGGGSGGGGSG |
| | | | | GGGSGGGGSGGGGSDKTHTCPPCPAPELLGGPSVFL |
| | | | | FPPKPKDTLMISRTPEVTCVVVDVSHEDPEVKFNWY |
| | | | | VDGVEVHNAKTKPCEEQYGSTYRCVSVLTVLHQDW |
| | | | | LNGKEYKCKVSNKALPAPIEKTISKAKGQPREPQVYTL |
| | | | | PPSREEMTKNQVSLTCLVKGFYPSDIAVEWESNGQP |
| | | | | ENNYKTTPPVLDSDGSFFLYSKLTVDKSRWQQGNVF |
| | | | | SCSVMHEALHNHYTQKSLSLSPGK |
| 100. | BCMA A7 27- | artificial | aa | NHIIH |
| | C4-G7 CDR1 VH | | | |
| 101. | BCMA A7 27- | artificial | aa | YINPYPGYHAYNEKFQG |
| | C4-G7 CDR2 VH | | | |
| 102. | BCMA A7 27- | artificial | aa | DGYYRDTDVLDY |
| | C4-G7 CDR3 VH | | | |
| 103. | BCMA A7 27- | artificial | aa | QASQDISNYLN |
| | C4-G7 CDR1 VL | | | |
| 104. | BCMA A7 27- | artificial | aa | YTSRLHT |
| | C4-G7 CDR2 VL | | | |
| 105. | BCMA A7 27- | artificial | aa | QQGNTLPWT |
| | C4-G7 CDR3 VL | | | |
| 106. | BCMA A7 27- | artificial | aa | QVQLVQSGAEVKKPGASVKVSCKASGYTFTNHIIHW |
| | C4-G7 CC | | | VRQAPGQCLEWMGYINPYPGYHAYNEKFQGRATM |
| | (44/100) VH | | | TSDTSTSTVYMELSSLRSEDTAVYYCARDGYYRDTDV |
| | | | | LDYWGQGTLVTVSS |
| 107. | BCMA A7 27- | artificial | aa | DIQMTQSPSSLSASVGDRVTITCQASQDISNYLNWY |
| | C4-G7 CC | | | QQKPGKAPKLLIYYTSRLHTGVPSRFSGSGSGTDFTFT |
| | (44/100) VL | | | ISSLEPEDIATYYCQQGNTLPWTFGCGTKLEIK |
| 108. | BCMA A7 27- | artificial | aa | QVQLVQSGAEVKKPGASVKVSCKASGYTFTNHIIHW |
| | C4-G7 CC | | | VRQAPGQCLEWMGYINPYPGYHAYNEKFQGRATM |
| | (44/100)scFv | | | TSDTSTSTVYMELSSLRSEDTAVYYCARDGYYRDTDV |
| | | | | LDYWGQGTLVTVSSGGGGSGGGGSGGGGSDIQMT |
| | | | | QSPSSLSASVGDRVTITCQASQDISNYLNWYQQKPG |
| | | | | KAPKLLIYYTSRLHTGVPSRFSGSGSGTDFTFTISSLEPE |
| | | | | DIATYYCQQGNTLPWTFGCGTKLEIK |
| 109. | BCMA A7 27- | artificial | aa | QVQLVQSGAEVKKPGASVKVSCKASGYTFTNHIIHW |
| | C4-G7 CC | | | VRQAPGQCLEWMGYINPYPGYHAYNEKFQGRATM |
| | (44/100) x I2C0 | | | TSDTSTSTVYMELSSLRSEDTAVYYCARDGYYRDTDV |
| | bispecific | | | LDYWGQGTLVTVSSGGGGSGGGGSGGGGSDIQMT |
| | molecule | | | QSPSSLSASVGDRVTITCQASQDISNYLNWYQQKPG |
| | | | | KAPKLLIYYTSRLHTGVPSRFSGSGSGTDFTFTISSLEPE |
| | | | | DIATYYCQQGNTLPWTFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVL |
| 110. | BCMA A7 27- | artificial | aa | QVQLVQSGAEVKKPGASVKVSCKASGYTFTNHIIHW |
| | C4-G7 CC | | | VRQAPGQCLEWMGYINPYPGYHAYNEKFQGRATM |
| | (44/100)x | | | TSDTSTSTVYMELSSLRSEDTAVYYCARDGYYRDTDV |
| | I2C0-scFc | | | LDYWGQGTLVTVSSGGGGSGGGGSGGGGSDIQMT |
| | bispecific | | | QSPSSLSASVGDRVTITCQASQDISNYLNWYQQKPG |
| | molecule HLE | | | KAPKLLIYYTSRLHTGVPSRFSGSGSGTDFTFTISSLEPE |
| | | | | DIATYYCQQGNTLPWTFGCGTKVEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVLGGG |
| | | | | GDKTHTCPPCPAPELLGGPSVFLFPPKPKDTLMISRTP |
| | | | | EVTCVVVDVSHEDPEVKFNWYVDGVEVHNAKTKPC |
| | | | | EEQYGSTYRCVSVLTVLHQDWLNGKEYKCKVSNKAL |
| | | | | PAPIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGKGGGGSGGGGSGGGGSGGGGSGGGG |
| | | | | SGGGGSDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGK |
| 111. | PM 76-B10.17 | artificial | aa | DYYMY |
| | CC VH CDR1 | | | |
| 112. | PM 76-B10.17 | artificial | aa | IISDAGYYTYYSDIIKG |
| | CC VH CDR2 | | | |
| 113. | PM 76-B10.17 | artificial | aa | GFPLLRHGAMDY |
| | CC VH CDR3 | | | |
| 114. | PM 76-B10.17 | artificial | aa | KASQNVDANVA |
| | CC VLCDR1 | | | |
| 145. | PM 76-B10.17 | artificial | aa | SASYVYW |
| | CC VLCDR2 | | | |
| 116. | PM 76-B10.17 | artificial | aa | QQYDQQLIT |
| | CC VLCDR3 | | | |
| 117. | PM 76-B10.17 | artificial | aa | QVQLVESGGGLVKPGESLRLSCAASGFTFSDYYMYW |
| | CC VH | | | VRQAPGKCLEWVAIISDAGYYTYYSDIIKGRFTISRDN |
| | | | | AKNSLYLQMNSLKAEDTAVYYCARGFPLLRHGAMD |
| | | | | YWGQGTLVTVSS |
| 118. | PM 76-B10.17 | artificial | aa | DIQMTQSPSSLSASVGDRVTITCKASQNVDANVAW |
| | CC VL | | | YQQKPGQAPKSLIYSASYVYWDVPSRFSGSASGTDFT |
| | | | | LTISSVQSEDFATYYCQQYDQQLITFGCGTKLEIK |
| 119. | PM 76-B10.17 | artificial | aa | QVQLVESGGGLVKPGESLRLSCAASGFTFSDYYMYW |
| | CC scFv | | | VRQAPGKCLEWVAIISDAGYYTYYSDIIKGRFTISRDN |
| | | | | AKNSLYLQMNSLKAEDTAVYYCARGFPLLRHGAMD |
| | | | | YWGQGTLVTVSSGGGGSGGGGSGGGGSDIQMTQS |
| | | | | PSSLSASVGDRVTITCKASQNVDANVAWYQQKPGQ |
| | | | | APKSLIYSASYVYWDVPSRFSGSASGTDFTLTISSVQS |
| | | | | EDFATYYCQQYDQQLITFGCGTKLEIK |
| 120. | PM 76-B10.17 | artificial | aa | QVQLVESGGGLVKPGESLRLSCAASGFTFSDYYMYW |
| | CC x I2C0 | | | VRQAPGKCLEWVAIISDAGYYTYYSDIIKGRFTISRDN |
| | bispecific | | | AKNSLYLQMNSLKAEDTAVYYCARGFPLLRHGAMD |
| | molecule | | | YWGQGTLVTVSSGGGGSGGGGSGGGGSDIQMTQS |
| | | | | PSSLSASVGDRVTITCKASQNVDANVAWYQQKPGQ |
| | | | | APKSLIYSASYVYWDVPSRFSGSASGTDFTLTISSVQS |
| | | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVL |
| 121. | PM 76-B10.17 | artificial | aa | QVQLVESGGGLVKPGESLRLSCAASGFTFSDYYMYW |
| | CC x I2C0-scFc | | | VRQAPGKCLEWVAIISDAGYYTYYSDIIKGRFTISRDN |
| | bispecific | | | AKNSLYLQMNSLKAEDTAVYYCARGFPLLRHGAMD |
| | HLE molecule | | | YWGQGTLVTVSSGGGGSGGGGSGGGGSDIQMTQS |
| | | | | PSSLSASVGDRVTITCKASQNVDANVAWYQQKPGQ |
| | | | | APKSLIYSASYVYWDVPSRFSGSASGTDFTLTISSVQS |
| | | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVLGGG |
| | | | | GDKTHTCPPCPAPELLGGPSVFLFPPKPKDTLMISRTP |
| | | | | EVTCVVVDVSHEDPEVKFNWYVDGVEVHNAKTKPC |
| | | | | EEQYGSTYRCVSVLTVLHQDWLNGKEYKCKVSNKAL |
| | | | | PAPIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGKGGGGSGGGGSGGGGSGGGGSGGGG |
| | | | | SGGGGSDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGK |
| 122. | PM 76-B10.17 | artificial | aa | QVQLVESGGGLVKPGESLRLSCAASGFTFSDYYMYW |
| | CC x I2C0- | | | VRQAPGKCLEWVAIISDAGYYTYYSDIIKGRFTISRDN |
| | scFc_delGK | | | AKNSLYLQMNSLKAEDTAVYYCARGFPLLRHGAMD |
| | bispecific | | | YWGQGTLVTVSSGGGGSGGGGSGGGGSDIQMTQS |
| | HLE molecule | | | PSSLSASVGDRVTITCKASQNVDANVAWYQQKPGQ |
| | | | | APKSLIYSASYVYWDVPSRFSGSASGTDFTLTISSVQS |
| | | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVLGGG |
| | | | | GDKTHTCPPCPAPELLGGPSVFLFPPKPKDTLMISRTP |
| | | | | EVTCVVVDVSHEDPEVKFNWYVDGVEVHNAKTKPC |
| | | | | EEQYGSTYRCVSVLTVLHQDWLNGKEYKCKVSNKAL |
| | | | | PAPIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGGGGSGGGGSGGGGSGGGGSGGGGSG |
| | | | | GGGSDKTHTCPPCPAPELLGGPSVFLFPPKPKDTLMI |
| | | | | SRTPEVTCVVVDVSHEDPEVKFNWYVDGVEVHNAK |
| | | | | TKPCEEQYGSTYRCVSVLTVLHQDWLNGKEYKCKVS |
| | | | | NKALPAPIEKTISKAKGQPREPQVYTLPPSREEMTKN |
| | | | | QVSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSPGK |
| 123. | PM 76-B10.17 | artificial | aa | QVQLVESGGGLVKPGESLRLSCAASGFTFSDYYMYW |
| | CC x I2CO CC | | | VRQAPGKCLEWVAIISDAGYYTYYSDIIKGRFTISRDN |
| | (103/43)-scFc | | | AKNSLYLQMNSLKAEDTAVYYCARGFPLLRHGAMD |
| | | | | YWGQGTLVTVSSGGGGSGGGGSGGGGSDIQMTQS |
| | | | | PSSLSASVGDRVTITCKASQNVDANVAWYQQKPGQ |
| | bispecific | | | APKSLIYSASYVYWDVPSRFSGSASGTDFTLTISSVQS |
| | molecule | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYCGQGTLVTVSSGGGGSGGGGSGGGGSQTVVT |
| | | | | QEPSLTVSPGGTVTLTCGSSTGAVTSGNYPNWVQQ |
| | | | | KPGQCPRGLIGGTKFLAPGTPARFSGSLLGGKAALTLS |
| | | | | GVQPEDEAEYYCVLWYSNRWVFGGGTKLTVL |
| 124. | PM 76-B10.17 | artificial | aa | QVQLVESGGGLVKPGESLRLSCAASGFTFSDYYMYW |
| | CC x I2C0 CC | | | VRQAPGKCLEWVAIISDAGYYTYYSDIIKGRFTISRDN |
| | (103/43)-scFc | | | AKNSLYLQMNSLKAEDTAVYYCARGFPLLRHGAMD |
| | bispecific | | | YWGQGTLVTVSSGGGGSGGGGSGGGGSDIQMTQS |
| | HLE molecule | | | PSSLSASVGDRVTITCKASQNVDANVAWYQQKPGQ |
| | | | | APKSLIYSASYVYWDVPSRFSGSASGTDFTLTISSVQS |
| | | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYCGQGTLVTVSSGGGGSGGGGSGGGGSQTVVT |
| | | | | QEPSLTVSPGGTVTLTCGSSTGAVTSGNYPNWVQQ |
| | | | | KPGQCPRGLIGGTKFLAPGTPARFSGSLLGGKAALTLS |
| | | | | GVQPEDEAEYYCVLWYSNRWVFGGGTKLTVLGGGG |
| | | | | DKTHTCPPCPAPELLGGPSVFLFPPKPKDTLMISRTPE |
| | | | | VTCVVVDVSHEDPEVKFNWYVDGVEVHNAKTKPCE |
| | | | | EQYGSTYRCVSVLTVLHQDWLNGKEYKCKVSNKALP |
| | | | | APIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGKGGGGSGGGGSGGGGSGGGGSGGGG |
| | | | | SGGGGSDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGK |
| 125. | PM 76-B10.17 | artificial | aa | QVQLVESGGGLVKPGESLRLSCAASGFTFSDYYMYW |
| | CC x I2CO CC | | | VRQAPGKCLEWVAIISDAGYYTYYSDIIKGRFTISRDN |
| | (103/43)- | | | AKNSLYLQMNSLKAEDTAVYYCARGFPLLRHGAMD |
| | scFc_delGK | | | YWGQGTLVTVSSGGGGSGGGGSGGGGSDIQMTQS |
| | bispecific | | | PSSLSASVGDRVTITCKASQNVDANVAWYQQKPGQ |
| | HLE molecule | | | APKSLIYSASYVYWDVPSRFSGSASGTDFTLTISSVQS |
| | | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYCGQGTLVTVSSGGGGSGGGGSGGGGSQTVVT |
| | | | | QEPSLTVSPGGTVTLTCGSSTGAVTSGNYPNWVQQ |
| | | | | KPGQCPRGLIGGTKFLAPGTPARFSGSLLGGKAALTLS |
| | | | | GVQPEDEAEYYCVLWYSNRWVFGGGTKLTVLGGGG |
| | | | | DKTHTCPPCPAPELLGGPSVFLFPPKPKDTLMISRTPE |
| | | | | VTCVVVDVSHEDPEVKFNWYVDGVEVHNAKTKPCE |
| | | | | EQYGSTYRCVSVLTVLHQDWLNGKEYKCKVSNKALP |
| | | | | APIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGGGGSGGGGSGGGGSGGGGSGGGGSG |
| | | | | GGGSDKTHTCPPCPAPELLGGPSVFLFPPKPKDTLMI |
| | | | | SRTPEVTCVVVDVSHEDPEVKFNWYVDGVEVHNAK |
| | | | | TKPCEEQYGSTYRCVSVLTVLHQDWLNGKEYKCKVS |
| | | | | NKALPAPIEKTISKAKGQPREPQVYTLPPSREEMTKN |
| | | | | QVSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSPGK |
| 126. | PM 76-B10.11 | artificial | aa | DYYMY |
| | CC VH CDR1 | | | |
| 127. | PM 76-B10.11 | artificial | aa | IISDGGYYTYYSDIIKG |
| | CC VH CDR2 | | | |
| 128. | PM 76-B10.11 | artificial | aa | GFPLLRHGAMDY |
| | CC VH CDR3 | | | |
| 129. | PM 76-B10.11 | artificial | aa | KASQNVDTNVA |
| | CC VL CDR1 | | | |
| 130. | PM 76-B10.11 | artificial | aa | SASYVYW |
| | CC VL CDR2 | | | |
| 131. | PM 76-B10.11 | artificial | aa | QQYDQQLIT |
| | CC VL CDR3 | | | |
| 132. | PM 76-B10.11 | artificial | aa | QVQLVESGGGLVKPGESLRLSCAASGFTFSDYYMYW |
| | CC VH | | | VRQAPGKGLEWVAIISDGGYYTYYSDIIKGRFTISRDN |
| | | | | AKNSLYLQMNSLKAEDTAVYYCARGFPLLRHGAMD |
| | | | | YWGQGTLVTVSS |
| 133. | PM 76-B10.11 | artificial | aa | DIQMTQSPSSLSASVGDRVTITCKASQNVDTNVAWY |
| | CC VL | | | QQKPGQAPKSLIYSASYVYWDVPSRFSGSASGTDFTL |
| | | | | TISSVQSEDFATYYCQQYDQQLITFGGGTKLEIK |
| 134. | PM 76-B10.11 | artificial | aa | QVQLVESGGGLVKPGESLRLSCAASGFTFSDYYMYW |
| | CC scFv | | | VRQAPGKGLEWVAIISDGGYYTYYSDIIKGRFTISRDN |
| | | | | AKNSLYLQMNSLKAEDTAVYYCARGFPLLRHGAMD |
| | | | | YWGQGTLVTVSSGGGGSGGGGSGGGGSDIQMTOS |
| | | | | PSSLSASVGDRVTITCKASQNVDTNVAWYQQKPGQ |
| | | | | APKSLIYSASYVYWDVPSRFSGSASGTDFTLTISSVQS |
| | | | | EDFATYYCQQYDQQLITFGGGTKLEIK |
| 135. | PM 76-B10.11 | artificial | aa | QVQLVESGGGLVKPGESLRLSCAASGFTFSDYYMYW |
| | CC x I2C0 | | | VRQAPGKGLEWVAIISDGGYYTYYSDIIKGRFTISRDN |
| | bispecific | | | AKNSLYLQMNSLKAEDTAVYYCARGFPLLRHGAMD |
| | molecule | | | YWGQGTLVTVSSGGGGSGGGGSGGGGSDIQMTQS |
| | | | | PSSLSASVGDRVTITCKASQNVDTNVAWYQQKPGQ |
| | | | | APKSLIYSASYVYWDVPSRFSGSASGTDFTLTISSVQS |
| | | | | EDFATYYCQQYDQQLITFGGGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVL |
| 136. | PM 76-B10.11 | artificial | aa | QVQLVESGGGLVKPGESLRLSCAASGFTFSDYYMYW |
| | CC x I2C0-scFc | | | VRQAPGKGLEWVAIISDGGYYTYYSDIIKGRFTISRDN |
| | bispecific | | | AKNSLYLQMNSLKAEDTAVYYCARGFPLLRHGAMD |
| | HLE molecule | | | YWGQGTLVTVSSGGGGSGGGGSGGGGSDIQMTQS |
| | | | | PSSLSASVGDRVTITCKASQNVDTNVAWYQQKPGQ |
| | | | | APKSLIYSASYVYWDVPSRFSGSASGTDFTLTISSVQS |
| | | | | EDFATYYCQQYDQQLITFGGGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVLGGG |
| | | | | GDKTHTCPPCPAPELLGGPSVFLFPPKPKDTLMISRTP |
| | | | | EVTCVVVDVSHEDPEVKFNWYVDGVEVHNAKTKPC |
| | | | | EEQYGSTYRCVSVLTVLHQDWLNGKEYKCKVSNKAL |
| | | | | PAPIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGKGGGGSGGGGSGGGGSGGGGSGGGG |
| | | | | SGGGGSDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGK |
| 137. | PM 76-B10.11 | artificial | aa | QVQLVESGGGLVKPGESLRLSCAASGFTFSDYYMYW |
| | CC x I2C0- | | | VRQAPGKGLEWVAIISDGGYYTYYSDIIKGRFTISRDN |
| | scFc_delGK | | | AKNSLYLQMNSLKAEDTAVYYCARGFPLLRHGAMD |
| | bispecific | | | YWGQGTLVTVSSGGGGSGGGGSGGGGSDIQMTQS |
| | HLE molecule | | | PSSLSASVGDRVTITCKASQNVDTNVAWYQQKPGQ |
| | | | | APKSLIYSASYVYWDVPSRFSGSASGTDFTLTISSVQS |
| | | | | EDFATYYCQQYDQQLITFGGGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVLGGG |
| | | | | GDKTHTCPPCPAPELLGGPSVFLFPPKPKDTLMISRTP |
| | | | | EVTCVVVDVSHEDPEVKFNWYVDGVEVHNAKTKPC |
| | | | | EEQYGSTYRCVSVLTVLHQDWLNGKEYKCKVSNKAL |
| | | | | PAPIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGGGGSGGGGSGGGGSGGGGSGGGGSG |
| | | | | GGGSDKTHTCPPCPAPELLGGPSVFLFPPKPKDTLMI |
| | | | | SRTPEVTCVVVDVSHEDPEVKFNWYVDGVEVHNAK |
| | | | | TKPCEEQYGSTYRCVSVLTVLHQDWLNGKEYKCKVS |
| | | | | NKALPAPIEKTISKAKGQPREPQVYTLPPSREEMTKN |
| | | | | QVSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSPGK |
| 138. | PM 76-B10.11 | artificial | aa | QVQLVESGGGLVKPGESLRLSCAASGFTFSDYYMYW |
| | CC x I2C0 CC | | | VRQAPGKGLEWVAIISDGGYYTYYSDIIKGRFTISRDN |
| | (103/43)-scFc | | | AKNSLYLQMNSLKAEDTAVYYCARGFPLLRHGAMD |
| | bispecific | | | YWGQGTLVTVSSGGGGSGGGGSGGGGSDIQMTQS |
| | molecule | | | PSSLSASVGDRVTITCKASQNVDTNVAWYQQKPGQ |
| | | | | APKSLIYSASYVYWDVPSRFSGSASGTDFTLTISSVQS |
| | | | | EDFATYYCQQYDQQLITFGGGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYCGQGTLVTVSSGGGGSGGGGSGGGGSQTVVT |
| | | | | QEPSLTVSPGGTVTLTCGSSTGAVTSGNYPNWVQQ |
| | | | | KPGQCPRGLIGGTKFLAPGTPARFSGSLLGGKAALTLS |
| | | | | GVQPEDEAEYYCVLWYSNRWVFGGGTKLTVL |
| 139. | PM 76-B10.11 | artificial | aa | QVQLVESGGGLVKPGESLRLSCAASGFTFSDYYMYW |
| | CC x I2C0 CC | | | VRQAPGKGLEWVAIISDGGYYTYYSDIIKGRFTISRDN |
| | (103/43)-scFc | | | AKNSLYLQMNSLKAEDTAVYYCARGFPLLRHGAMD |
| | bispecific | | | YWGQGTLVTVSSGGGGSGGGGSGGGGSDIQMTQS |
| | HLE molecule | | | PSSLSASVGDRVTITCKASQNVDTNVAWYQQKPGQ |
| | | | | APKSLIYSASYVYWDVPSRFSGSASGTDFTLTISSVQS |
| | | | | EDFATYYCQQYDQQLITFGGGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYCGQGTLVTVSSGGGGSGGGGSGGGGSQTVVT |
| | | | | QEPSLTVSPGGTVTLTCGSSTGAVTSGNYPNWVQQ |
| | | | | KPGQCPRGLIGGTKFLAPGTPARFSGSLLGGKAALTLS |
| | | | | GVQPEDEAEYYCVLWYSNRWVFGGGTKLTVLGGGG |
| | | | | DKTHTCPPCPAPELLGGPSVFLFPPKPKDTLMISRTPE |
| | | | | VTCVVVDVSHEDPEVKFNWYVDGVEVHNAKTKPCE |
| | | | | EQYGSTYRCVSVLTVLHQDWLNGKEYKCKVSNKALP |
| | | | | APIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGKGGGGSGGGGSGGGGSGGGGSGGGG |
| | | | | SGGGGSDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGK |
| 140. | PM 76-B10.11 | artificial | aa | QVQLVESGGGLVKPGESLRLSCAASGFTFSDYYMYW |
| | CC x I2C0 CC | | | VRQAPGKGLEWVAIISDGGYYTYYSDIIKGRFTISRDN |
| | (103/43)- | | | AKNSLYLQMNSLKAEDTAVYYCARGFPLLRHGAMD |
| | scFc_delGK | | | YWGQGTLVTVSSGGGGSGGGGSGGGGSDIQMTQS |
| | bispecific | | | PSSLSASVGDRVTITCKASQNVDTNVAWYQQKPGQ |
| | HLE molecule | | | APKSLIYSASYVYWDVPSRFSGSASGTDFTLTISSVQS |
| | | | | EDFATYYCQQYDQQLITFGGGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYCGQGTLVTVSSGGGGSGGGGSGGGGSQTVVT |
| | | | | QEPSLTVSPGGTVTLTCGSSTGAVTSGNYPNWVQQ |
| | | | | KPGQCPRGLIGGTKFLAPGTPARFSGSLLGGKAALTLS |
| | | | | GVQPEDEAEYYCVLWYSNRWVFGGGTKLTVLGGGG |
| | | | | DKTHTCPPCPAPELLGGPSVFLFPPKPKDTLMISRTPE |
| | | | | VTCVVVDVSHEDPEVKFNWYVDGVEVHNAKTKPCE |
| | | | | EQYGSTYRCVSVLTVLHQDWLNGKEYKCKVSNKALP |
| | | | | APIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGGGGSGGGGSGGGGSGGGGSGGGGSG |
| | | | | GGGSDKTHTCPPCPAPELLGGPSVFLFPPKPKDTLMI |
| | | | | SRTPEVTCVVVDVSHEDPEVKFNWYVDGVEVHNAK |
| | | | | TKPCEEQYGSTYRCVSVLTVLHQDWLNGKEYKCKVS |
| | | | | NKALPAPIEKTISKAKGQPREPQVYTLPPSREEMTKN |
| | | | | QVSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSPGK |
| 141. | PM 76-B10.11 | artificial | aa | DYYMY |
| | CC x I2C0-scFc | | | |
| | VH CDR1 | | | |
| 142. | PM 76-B10.11 | artificial | aa | IISDGGYYTYYSDIIKG |
| | CC x I2C0-scFc | | | |
| | VH CDR2 | | | |
| 143. | PM 76-B10.11 | artificial | aa | GFPLLRHGAMDY |
| | CC x I2C0-scFc | | | |
| | VH CDR3 | | | |
| 144. | PM 76-B10.11 | artificial | aa | KASQNVDTNVA |
| | CC x I2C0-scFc | | | |
| | VLCDR1 | | | |
| 145. | PM 76-B10.11 | artificial | aa | SASYVYW |
| | CC x I2C0-scFc | | | |
| | VLCDR2 | | | |
| 146. | PM 76-B10.11 | artificial | aa | QQYDQQLIT |
| | CC x I2C0-scFc | | | |
| | VLCDR3 | | | |
| 147. | PM 76-B10.11 | artificial | aa | QVQLVESGGGLVKPGESLRLSCAASGFTFSDYYMYW |
| | CC x I2C0-scFc | | | VRQAPGKCLEWVAIISDGGYYTYYSDIIKGRFTISRDN |
| | VH | | | AKNSLYLQMNSLKAEDTAVYYCARGFPLLRHGAMD |
| | | | | YWGQGTLVTVSS |
| 148. | PM 76-B10.11 | artificial | aa | DIQMTQSPSSLSASVGDRVTITCKASQNVDTNVAWY |
| | CC x I2C0-scFc | | | QQKPGQAPKSLIYSASYVYWDVPSRFSGSASGTDFTL |
| | VL | | | TISSVQSEDFATYYCQQYDQQLITFGCGTKLEIK |
| 149. | PM 76-B10.11 | artificial | aa | QVQLVESGGGLVKPGESLRLSCAASGFTFSDYYMYW |
| | CC x I2C0-scFc | | | VRQAPGKCLEWVAIISDGGYYTYYSDIIKGRFTISRDN |
| | scFv | | | AKNSLYLQMNSLKAEDTAVYYCARGFPLLRHGAMD |
| | | | | YWGQGTLVTVSSGGGGSGGGGSGGGGSDIQMTQS |
| | | | | PSSLSASVGDRVTITCKASQNVDTNVAWYQQKPGQ |
| | | | | APKSLIYSASYVYWDVPSRFSGSASGTDFTLTISSVQS |
| | | | | EDFATYYCQQYDQQLITFGCGTKLEIK |
| 150. | PM 76-B10.11 | artificial | aa | QVQLVESGGGLVKPGESLRLSCAASGFTFSDYYMYW |
| | CC x I2C0-scFc | | | VRQAPGKCLEWVAIISDGGYYTYYSDIIKGRFTISRDN |
| | bispecific | | | AKNSLYLQMNSLKAEDTAVYYCARGFPLLRHGAMD |
| | molecule | | | YWGQGTLVTVSSGGGGSGGGGSGGGGSDIQMTQS |
| | | | | PSSLSASVGDRVTITCKASQNVDTNVAWYQQKPGQ |
| | | | | APKSLIYSASYVYWDVPSRFSGSASGTDFTLTISSVQS |
| | | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVL |
| 151. | PM 76-B10.11 | artificial | aa | QVQLVESGGGLVKPGESLRLSCAASGFTFSDYYMYW |
| | CC x I2C0-scFc | | | VRQAPGKCLEWVAIISDGGYYTYYSDIIKGRFTISRDN |
| | bispecific | | | AKNSLYLQMNSLKAEDTAVYYCARGFPLLRHGAMD |
| | HLE molecule | | | YWGQGTLVTVSSGGGGSGGGGSGGGGSDIQMTQS |
| | | | | PSSLSASVGDRVTITCKASQNVDTNVAWYQQKPGQ |
| | | | | APKSLIYSASYVYWDVPSRFSGSASGTDFTLTISSVQS |
| | | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVLGGG |
| | | | | GDKTHTCPPCPAPELLGGPSVFLFPPKPKDTLMISRTP |
| | | | | EVTCVVVDVSHEDPEVKFNWYVDGVEVHNAKTKPC |
| | | | | EEQYGSTYRCVSVLTVLHQDWLNGKEYKCKVSNKAL |
| | | | | PAPIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGKGGGGSGGGGSGGGGSGGGGSGGGG |
| | | | | SGGGGSDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGK |
| 152. | PM 76-B10.11 | artificial | aa | QVQLVESGGGLVKPGESLRLSCAASGFTFSDYYMYW |
| | CC x I2C0- | | | VRQAPGKCLEWVAIISDGGYYTYYSDIIKGRFTISRDN |
| | scFc_delGK | | | AKNSLYLQMNSLKAEDTAVYYCARGFPLLRHGAMD |
| | bispecific | | | YWGQGTLVTVSSGGGGSGGGGSGGGGSDIQMTQS |
| | HLE molecule | | | PSSLSASVGDRVTITCKASQNVDTNVAWYQQKPGQ |
| | | | | APKSLIYSASYVYWDVPSRFSGSASGTDFTLTISSVQS |
| | | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVLGGG |
| | | | | GDKTHTCPPCPAPELLGGPSVFLFPPKPKDTLMISRTP |
| | | | | EVTCVVVDVSHEDPEVKFNWYVDGVEVHNAKTKPC |
| | | | | EEQYGSTYRCVSVLTVLHQDWLNGKEYKCKVSNKAL |
| | | | | PAPIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGGGGSGGGGSGGGGSGGGGSGGGGSG |
| | | | | GGGSDKTHTCPPCPAPELLGGPSVFLFPPKPKDTLMI |
| | | | | SRTPEVTCVVVDVSHEDPEVKFNWYVDGVEVHNAK |
| | | | | TKPCEEQYGSTYRCVSVLTVLHQDWLNGKEYKCKVS |
| | | | | NKALPAPIEKTISKAKGQPREPQVYTLPPSREEMTKN |
| | | | | QVSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSPGK |
| 153. | PM 76-B10.11 | artificial | aa | QVQLVESGGGLVKPGESLRLSCAASGFTFSDYYMYW |
| | CC x I2C0 CC | | | VRQAPGKCLEWVAIISDGGYYTYYSDIIKGRFTISRDN |
| | (103/43)-scFc | | | AKNSLYLQMNSLKAEDTAVYYCARGFPLLRHGAMD |
| | bispecific | | | YWGQGTLVTVSSGGGGSGGGGSGGGGSDIQMTQS |
| | molecule | | | PSSLSASVGDRVTITCKASQNVDTNVAWYQQKPGQ |
| | | | | APKSLIYSASYVYWDVPSRFSGSASGTDFTLTISSVQS |
| | | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYCGQGTLVTVSSGGGGSGGGGSGGGGSQTVVT |
| | | | | QEPSLTVSPGGTVTLTCGSSTGAVTSGNYPNWVQQ |
| | | | | KPGQCPRGLIGGTKFLAPGTPARFSGSLLGGKAALTLS |
| | | | | GVQPEDEAEYYCVLWYSNRWVFGGGTKLTVL |
| 154. | PM 76-B10.11 | artificial | aa | QVQLVESGGGLVKPGESLRLSCAASGFTFSDYYMYW |
| | CC x I2C0 CC | | | VRQAPGKCLEWVAIISDGGYYTYYSDIIKGRFTISRDN |
| | (103/43)-scFc | | | AKNSLYLQMNSLKAEDTAVYYCARGFPLLRHGAMD |
| | bispecific | | | YWGQGTLVTVSSGGGGSGGGGSGGGGSDIQMTQS |
| | HLE molecule | | | PSSLSASVGDRVTITCKASQNVDTNVAWYQQKPGQ |
| | | | | APKSLIYSASYVYWDVPSRFSGSASGTDFTLTISSVQS |
| | | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYCGQGTLVTVSSGGGGSGGGGSGGGGSQTVVT |
| | | | | QEPSLTVSPGGTVTLTCGSSTGAVTSGNYPNWVQQ |
| | | | | KPGQCPRGLIGGTKFLAPGTPARFSGSLLGGKAALTLS |
| | | | | GVQPEDEAEYYCVLWYSNRWVFGGGTKLTVLGGGG |
| | | | | DKTHTCPPCPAPELLGGPSVFLFPPKPKDTLMISRTPE |
| | | | | VTCVVVDVSHEDPEVKFNWYVDGVEVHNAKTKPCE |
| | | | | EQYGSTYRCVSVLTVLHQDWLNGKEYKCKVSNKALP |
| | | | | APIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGKGGGGSGGGGSGGGGSGGGGSGGGG |
| | | | | SGGGGSDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGK |
| 155. | PM 76-B10.11 | artificial | aa | QVQLVESGGGLVKPGESLRLSCAASGFTFSDYYMYW |
| | CC x I2C0 CC | | | VRQAPGKCLEWVAIISDGGYYTYYSDIIKGRFTISRDN |
| | (103/43)- | | | AKNSLYLQMNSLKAEDTAVYYCARGFPLLRHGAMD |
| | scFc_delGK | | | YWGQGTLVTVSSGGGGSGGGGSGGGGSDIQMTQS |
| | bispecific | | | PSSLSASVGDRVTITCKASQNVDTNVAWYQQKPGQ |
| | HLE molecule | | | APKSLIYSASYVYWDVPSRFSGSASGTDFTLTISSVQS |
| | | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYCGQGTLVTVSSGGGGSGGGGSGGGGSQTVVT |
| | | | | QEPSLTVSPGGTVTLTCGSSTGAVTSGNYPNWVQQ |
| | | | | KPGQCPRGLIGGTKFLAPGTPARFSGSLLGGKAALTLS |
| | | | | GVQPEDEAEYYCVLWYSNRWVFGGGTKLTVLGGGG |
| | | | | DKTHTCPPCPAPELLGGPSVFLFPPKPKDTLMISRTPE |
| | | | | VTCVVVDVSHEDPEVKFNWYVDGVEVHNAKTKPCE |
| | | | | EQYGSTYRCVSVLTVLHQDWLNGKEYKCKVSNKALP |
| | | | | APIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGGGGSGGGGSGGGGSGGGGSGGGGSG |
| | | | | GGGSDKTHTCPPCPAPELLGGPSVFLFPPKPKDTLMI |
| | | | | SRTPEVTCVVVDVSHEDPEVKFNWYVDGVEVHNAK |
| | | | | TKPCEEQYGSTYRCVSVLTVLHQDWLNGKEYKCKVS |
| | | | | NKALPAPIEKTISKAKGQPREPQVYTLPPSREEMTKN |
| | | | | QVSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSPGK |
| 156. | CD70_21D_CC | artificial | aa | TYAMS |
| | VH CDR1 | | | |
| 157. | CD70_21D_CC | artificial | aa | AISGSGGRTFYAESVEG |
| | VH CDR2 | | | |
| 158. | CD70_21D_CC | artificial | aa | HDYSNYPYFDY |
| | VH CDR3 | | | |
| 1659 | CD70_21D_CC | artificial | aa | RASQSVRSTYLA |
| | VL CDR1 | | | |
| 160. | CD70_21D_CC | artificial | aa | GASSRAT |
| | VL CDR2 | | | |
| 161. | CD70_21D_CC | artificial | aa | QQYGDLPFT |
| | VL CDR3 | | | |
| 162. | CD70_21D_CC | artificial | aa | EVQLLESGGGMVQPGGSLRLSCAASGFTFSTYAMS |
| | VH | | | WVRQAPGKCLEWVSAISGSGGRTFYAESVEGRFTIS |
| | | | | RDNSKNTLYLQMNSLRAEDTAVYYCAKHDYSNYPYF |
| | | | | DYWGQGTLVTVSS |
| 163. | CD70_21D_CC | artificial | aa | EIVLTQSPGTLSLSPGERATLSCRASQSVRSTYLAWYQ |
| | VL | | | QK |
| | | | | PGQAPRLLIYGASSRATGIPDRFSGSGSGTDFTLTISRL |
| | | | | E |
| | | | | PEDFAVYSCQQYGDLPFTFGCGTKLEIK |
| 164. | CD70_21D_CCx | artificial | aa | EVQLLESGGGMVQPGGSLRLSCAASGFTFSTYAMS |
| | 12C scFc | | | WVRQAPGKCLEWVSAISGSGGRTFYAESVEGRFTIS |
| | | | | RDNSKNTLYLQMNSLRAEDTAVYYCAKHDYSNYPYF |
| | | | | DYWGQGTLVTVSSGGGGSGGGGSGGGGSEIVLTQS |
| | | | | PGTLSLSPGERATLSCRASQSVRSTYLAWYQQKPGQ |
| | | | | APRLLIYGASSRATGIPDRFSGSGSGTDFTLTISRLEPE |
| | | | | DFAVYSCQQYGDLPFTFGCGTKLEIKSGGGGSEVQLV |
| | | | | ESGGGLVQPGGSLKLSCAASGFTFNKYAMNWVRQA |
| | | | | PGKGLEWVARIRSKYNNYATYYADSVKDRFTISRDDS |
| | | | | KNTAYLQMNNLKTEDTAVYYCVRHGNFGNSYISYW |
| | | | | AYWGQGTLVTVSSGGGGSGGGGSGGGGSQTVVTQ |
| | | | | EPSLTVSPGGTVTLTCGSSTGAVTSGNYPNWVQQKP |
| | | | | GQAPRGLIGGTKFLAPGTPARFSGSLLGGKAALTLSG |
| | | | | VQPEDEAEYYCVLWYSNRWVFGGGTKLTVLGGGGD |
| | | | | KTHTCPPCPAPELLGGPSVFLFPPKPKDTLMISRTPEV |
| | | | | TCVVVDVSHEDPEVKFNWYVDGVEVHNAKTKPCEE |
| | | | | QYGSTYRCVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSF |
| | | | | FLYSKLTVDKSRWQQGNVFSCSVMHEALHNHYTQK |
| | | | | SLSLSPGKGGGGSGGGGSGGGGSGGGGSGGGGSG |
| | | | | GGGSDKTHTCPPCPAPELLGGPSVFLFPPKPKDTLMI |
| | | | | SRTPEVTCVVVDVSHEDPEVKFNWYVDGVEVHNAK |
| | | | | TKPCEEQYGSTYRCVSVLTVLHQDWLNGKEYKCKVS |
| | | | | NKALPAPIEKTISKAKGQPREPQVYTLPPSREEMTKN |
| | | | | QVSLTCLVKGFYPSDIAVEWESNGQPENNYKTTPPVL |
| | | | | DSDGSFFLYSKLTVDKSRWQQGNVFSCSVMHEALH |
| | | | | NHYTQKSLSLSPGK |
| 165 | MSLN_5_CCxC | | | MGWSCIILFLVATATGVHSQVQLVESGGGLVKPGGS |
| | D3-scFc | | | LRLSCAASGFTFSDYYMTWIRQAPGKGLEWLSYISSS |
| | Bispecific HLE | | | GSTIYYADSVKGRFTISRDNAKNSLFLQMNSLRAEDT |
| | molecule | | | AVYYCARDRNSHFDYWGQGTLVTVSSGGGGSGGG |
| | | | | GSGGGGSDIQMTQSPSSVSASVGDRVTITCRASQGI |
| | | | | NTWLAWYQQKPGKAPKLLIYGASGLQSGVPSRFSGS |
| | | | | GSGTDFTLTISSLQPEDFATYYCQQAKSFPRTFGQGT |
| | | | | KVEIKSGGGGSEVQLVESGGGLVQPGGSLKLSCAAS |
| | | | | GFTFNKYAMNWVRQAPGKGLEWVARIRSKYNNYA |
| | | | | TYYADSVKDRFTISRDDSKNTAYLQMNNLKTEDTAV |
| | | | | YYCVRHGNFGNSYISYWAYWGQGTLVTVSSGGGGS |
| | | | | GGGGSGGGGSQTVVTQEPSLTVSPGGTVTLTCGSST |
| | | | | GAVTSGNYPNWVQQKPGQAPRGLIGGTKFLAPGTP |
| | | | | ARFSGSLLGGKAALTLSGVQPEDEAEYYCVLWYSNR |
| | | | | WVFGGGTKLTVLGGGGDKTHTCPPCPAPELLGGPSV |
| | | | | FLFPPKPKDTLMISRTPEVTCVVVDVSHEDPEVKFNW |
| | | | | YVDGVEVHNAKTKPCEEQYGSTYRCVSVLTVLHQD |
| | | | | WLNGKEYKCKVSNKALPAPIEKTISKAKGQPREPQVY |
| | | | | TLPPSREEMTKNQVSLTCLVKGFYPSDIAVEWESNG |
| | | | | QPENNYKTTPPVLDSDGSFFLYSKLTVDKSRWQQGN |
| | | | | VFSCSVMHEALHNHYTQKSLSLSPGKGGGGSGGGG |
| | | | | SGGGGSGGGGSGGGGSGGGGSDKTHTCPPCPAPEL |
| | | | | LGGPSVFLFPPKPKDTLMISRTPEVTCVVVDVSHEDP |
| | | | | EVKFNWYVDGVEVHNAKTKPCEEQYGSTYRCVSVLT |
| | | | | VLHQDWLNGKEYKCKVSNKALPAPIEKTISKAKGQPR |
| | | | | EPQVYTLPPSREEMTKNQVSLTCLVKGFYPSDIAVEW |
| | | | | ESNGQPENNYKTTPPVLDSDGSFFLYSKLTVDKSRW |
| | | | | QQGNVFSCSVMHEALHNHYTQKSLSLSPGK |
| 166 | Anti-PSMA | | | QVQLVESGGGLVKPGESLRLSCAASGFTFSDYYMYW |
| | xI2C0 with cys- | | | VRQAPGKCLEWVAIISDGGYYTYYSDIIKGRFTISRDN |
| | clamp, scFc | | | AKNSLYLQMNSLKAEDTAVYYCARGFPLLRHGAMD |
| | Bispecific | | | YWGQGTLVTVSSGGGGSGGGGSGGGGSDIQMTQS |
| | molecule HLE | | | PSSLSASVGDRVTITCKASQNVDTNVAWYQQKPGQ |
| | PM76-B10.11 | | | APKSLIYSASYVYWDVPSRFSGSASGTDFTLTISSVQS |
| | | | | EDFATYYCQQYDQQLITFGCGTKLEIKSGGGGSEVQL |
| | | | | VESGGGLVQPGGSLKLSCAASGFTFNKYAMNWVRQ |
| | | | | APGKGLEWVARIRSKYNNYATYYADSVKDRFTISRDD |
| | | | | SKNTAYLQMNNLKTEDTAVYYCVRHGNFGNSYISY |
| | | | | WAYWGQGTLVTVSSGGGGSGGGGSGGGGSQTVV |
| | | | | TQEPSLTVSPGGTVTLTCGSSTGAVTSGNYPNWVQQ |
| | | | | KPGQAPRGLIGGTKFLAPGTPARFSGSLLGGKAALTL |
| | | | | SGVQPEDEAEYYCVLWYSNRWVFGGGTKLTVLGGG |
| | | | | GDKTHTCPPCPAPELLGGPSVFLFPPKPKDTLMISRTP |
| | | | | EVTCVVVDVSHEDPEVKFNWYVDGVEVHNAKTKPC |
| | | | | EEQYGSTYRCVSVLTVLHQDWLNGKEYKCKVSNKAL |
| | | | | PAPIEKTISKAKGQPREPQVYTLPPSREEMTKNQVSLT |
| | | | | CLVKGFYPSDIAVEWESNGQPENNYKTTPPVLDSDG |
| | | | | SFFLYSKLTVDKSRWQQGNVFSCSVMHEALHNHYT |
| | | | | QKSLSLSPGKGGGGSGGGGSGGGGSGGGGSGGGG |
| | | | | SGGGGSDKTHTCPPCPAPELLGGPSVFLFPPKPKDTL |
| | | | | MISRTPEVTCVVVDVSHEDPEVKFNWYVDGVEVHN |
| | | | | AKTKPCEEQYGSTYRCVSVLTVLHQDWLNGKEYKCK |
| | | | | VSNKALPAPIEKTISKAKGQPREPQVYTLPPSREEMTK |
| | | | | NQVSLTCLVKGFYPSDIAVEWESNGQPENNYKTTPP |
| | | | | VLDSDGSFFLYSKLTVDKSRWQQGNVFSCSVMHEAL |
| | | | | HNHYTQKSLSLSPGK |
| 167 | Anti-PSMA | | | QVQLVESGGGLVKPGESLRLSCAASGFTFSDYYMYW |
| | IC20 bispecific | | | VRQAPGKGLEWVAIISDGGYYTYYSDIIKGRFTISRDN |
| | molecule | | | AKNSLYLQMNSLKAEDTAVYYCARGFPLLRHGAMD |
| | PM76-B10.17 | | | YWGQGTLVTVSSGGGGSGGGGSGGGGSDIQMTQS |
| | | | | PSSLSASVGDRVTITCKASQNVDTNVAWYQQKPGQ |
| | | | | APKSLIYSASYRYSDVPSRFSGSASGTDFTLTISSVQSE |
| | | | | DFATYYCQQYDSYPYTFGGGTKLEIKSGGGGSEVQLV |
| | | | | ESGGGLVQPGGSLKLSCAASGFTFNKYAMNWVRQA |
| | | | | PGKGLEWVARIRSKYNNYATYYADSVKDRFTISRDDS |
| | | | | KNTAYLQMNNLKTEDTAVYYCVRHGNFGNSYISYW |
| | | | | AYWGQGTLVTVSSGGGGSGGGGSGGGGSQTVVTQ |
| | | | | EPSLTVSPGGTVTLTCGSSTGAVTSGNYPNWVQQKP |
| | | | | GQAPRGLIGGTKFLAPGTPARFSGSLLGGKAALTLSG |
| | | | | VQPEDEAEYYCVLWYSNRWVFGGGTKLTVL |
| 168 | Anti-Cldn 18.2 | artificial | aa | GYYMH |
| | VH CDR1 | | | |
| | CL-1 and CL-2 | | | |
| 169 | Anti-Cldn18.2 | artificial | aa | WINPNSGGTKYAQKFQG |
| | VH CDR2 | | | |
| 170 | Anti-Cldn18.2 | artificial | aa | DRITVAGTYYYYGMDV |
| | VH CDR3 | | | |
| 171 | Anti-Cldn18.2 | artificial | aa | RASQGVNNWLA |
| | VLCDR1 | | | |
| 172 | Anti-Cldn18.2 | artificial | aa | TASSLQS |
| | VLCDR2 | | | |
| 173 | Anti-Cldn18.2 | artificial | aa | QQANSFPIT |
| | VLCDR3 | | | |
| 174 | Anti-Cldn18.2 | artificial | aa | QVQLVQSGAEVKKPGASVKVSCKASGYTFTGYYMHWVR |
| | VH anti- | | | QAPGQCLEWMGWINPNSGGTKYAQKFQGRVTMTRDT |
| | CL-1 | | | SISTAYMELSRLRSDDTAVYYCARDRITVAGTYYYYGMDV |
| | | | | WGQGTTVTVSS |
| 175 | Anti-Cldn18.2 | artificial | aa | DIQMTQSPSSVSASVGDRVTITCRASQGVNNWLAWYQQ |
| | VL | | | KPGKAPKLLIYTASSLQSGVPSRFSGSGSGTDFTLTIRSLQP |
| | CL-1 | | | EDFATYYCQQANSFPITFGCGTRLEIK |
| 176 | Anti-Cldn18.2 | artificial | aa | QVQLVQSGAEVKKPGASVKVSCKASGYTFTGYYMHWVR |
| | scFv | | | QAPGQCLEWMGWINPNSGGTKYAQKFQGRVTMTRDT |
| | CL-1 | | | SISTAYMELSRLRSDDTAVYYCARDRITVAGTYYYYGMDV |
| | | | | WGQGTTVTVSSGGGGSGGGGSGGGGSDIQMTQSPSSV |
| | | | | SASVGDRVTITCRASQGVNNWLAWYQQKPGKAPKLLIYT |
| | | | | ASSLQSGVPSRFSGSGSGTDFTLTIRSLQPEDFATYYCQQA |
| | | | | NSFPITFGCGTRLEIK |
| 177 | Anti-Cldn | artificial | aa | QVQLVQSGAEVKKPGASVKVSCKASGYTFTGYYMHWVR |
| | 18.2xCD3 | | | QAPGQCLEWMGWINPNSGGTKYAQKFQGRVTMTRDT |
| | bispecific | | | SISTAYMELSRLRSDDTAVYYCARDRITVAGTYYYYGMDV |
| | molecule | | | WGQGTTVTVSSGGGGSGGGGSGGGGSDIQMTQSPSSV |
| | CL-1 xI2C | | | SASVGDRVTITCRASQGVNNWLAWYQQKPGKAPKLLIYT |
| | | | | ASSLQSGVPSRFSGSGSGTDFTLTIRSLQPEDFATYYCQQA |
| | | | | NSFPITFGCGTRLEIKSGGGGSEVQLVESGGGLVQPGGSLK |
| | | | | LSCAASGFTFNKYAMNWVRQAPGKGLEWVARIRSKYNN |
| | | | | YATYYADSVKDRFTISRDDSKNTAYLQMNNLKTEDTAVYY |
| | | | | CVRHGNFGNSYISYWAYWGQGTLVTVSSGGGGSGGGGS |
| | | | | GGGGSQTVVTQEPSLTVSPGGTVTLTCGSSTGAVTSGNY |
| | | | | PNWVQQKPGQAPRGLIGGTKFLAPGTPARFSGSLLGGKA |
| | | | | ALTLSGVQPEDEAEYYCVLWYSNRWVFGGGTKLTVL |
| 178 | Anti-Cldn | artificial | aa | QVQLVQSGAEVKKPGASVKVSCKASGYTFTGYYMHWVR |
| | 18.2xCD3 | | | QAPGQCLEWMGWINPNSGGTKYAQKFQGRVTMTRDT |
| | Bispecific scFc | | | SISTAYMELSRLRSDDTAVYYCARDRITVAGTYYYYGMDV |
| | molecule | | | WGQGTTVTVSSGGGGSGGGGSGGGGSDIQMTQSPSSV |
| | CL-1 xI2C-scFc | | | SASVGDRVTITCRASQGVNNWLAWYQQKPGKAPKLLIYT |
| | | | | ASSLQSGVPSRFSGSGSGTDFTLTIRSLQPEDFATYYCQQA |
| | | | | NSFPITFGCGTRLEIKSGGGGSEVQLVESGGGLVQPGGSLK |
| | | | | LSCAASGFTFNKYAMNWVRQAPGKGLEWVARIRSKYNN |
| | | | | YATYYADSVKDRFTISRDDSKNTAYLQMNNLKTEDTAVYY |
| | | | | CVRHGNFGNSYISYWAYWGQGTLVTVSSGGGGSGGGGS |
| | | | | GGGGSQ.TVVTQEPSLTVSPGGTVTLTCGSSTGAVTSGNY |
| | | | | PNWVQQKPGQAPRGLIGGTKFLAPGTPARFSGSLLGGKA |
| | | | | ALTLSGVQPEDEAEYYCVLWYSNRWVFGGGTKLTVLGGG |
| | | | | GDKTHTCPPCPAPELLGGPSVFLFPPKPKDTLMISRTPEVT |
| | | | | CVVVDVSHEDPEVKFNWYVDGVEVHNAKTKPCEEQYGS |
| | | | | TYRCVSVLTVLHQDWLNGKEYKCKVSNKALPAPIEKTISKA |
| | | | | KGQPREPQVYTLPPSREEMTKNQVSLTCLVKGFYPSDIAV |
| | | | | EWESNGQPENNYKTTPPVLDSDGSFFLYSKLTVDKSRWQ. |
| | | | | QGNVFSCSVMHEALHNHYTQKSLSLSPGKGGGGSGGGG |
| | | | | SGGGGSGGGGSGGGGSGGGGSDKTHTCPPCPAPELLGG |
| | | | | PSVFLFPPKPKDTLMISRTPEVTCVVVDVSHEDPEVKFNW |
| | | | | YVDGVEVHNAKTKPCEEQYGSTYRCVSVLTVLHQDWLNG |
| | | | | KEYKCKVSNKALPAPIEKTISKAKGQPREPQVYTLPPSREE |
| | | | | MTKNQVSLTCLVKGFYPSDIAVEWESNGQPENNYKTTPP |
| | | | | VLDSDGSFFLYSKLTVDKSRWQQGNVFSCSVMHEALHNH |
| | | | | YTQKSLSLSPGK |
| 179 | Anti-Cldn18.2 | artificial | aa | QVQMVQSGAEVKKHGASVKVSCKASGYTFTGYYMHWV |
| | VH | | | RQAPGQCLEWMGWINPNSGGTKYAQKFQGRVTMTRD |
| | CL-2 | | | TSISTAYMELSRLRSDDTAVYYCARDRITVAGTYYYYGMD |
| | | | | VWGQGTTVTVSS |
| 180 | Anti-Cldn18.2 | artificial | aa | DIQMTQSPSSVSASVGDRVTITCRASQGVNNWLAWYQQ |
| | VL | | | KPGKAPKLLIYTASSLQSGVPSRFSGSGSGTDFTLTIRSLQP |
| | CL-2 | | | EDFATYYCQQANSFPITFGCGTRLEIK |
| 181 | Anti-Cldn18.2 | artificial | aa | QVQMVQSGAEVKKHGASVKVSCKASGYTFTGYYMHWV |
| | scFv | | | RQAPGQCLEWMGWINPNSGGTKYAQKFQGRVTMTRD |
| | CL-2 | | | TSISTAYMELSRLRSDDTAVYYCARDRITVAGTYYYYGMD |
| | | | | VWGQGTTVTVSSGGGGSGGGGSGGGGSDIQMTQSPSS |
| | | | | VSASVGDRVTITCRASQGVNNWLAWYQQKPGKAPKLLIY |
| | | | | TASSLQSGVPSRFSGSGSGTDFTLTIRSLQPEDFATYYCQQ |
| | | | | ANSFPITFGCGTRLEIK |
| 182 | Anti- | artificial | aa | QVQMVQSGAEVKKHGASVKVSCKASGYTFTGYYMHWV |
| | Cldn18.2xCD3 | | | RQAPGQCLEWMGWINPNSGGTKYAQKFQGRVTMTRD |
| | bispecific | | | TSISTAYMELSRLRSDDTAVYYCARDRITVAGTYYYYGMD |
| | molecule | | | VWGQGTTVTVSSGGGGSGGGGSGGGGSDIQMTQSPSS |
| | CL-2xI2C | | | VSASVGDRVTITCRASQGVNNWLAWYQQKPGKAPKLLIY |
| | | | | TASSLQSGVPSRFSGSGSGTDFTLTIRSLQPEDFATYYCQQ |
| | | | | ANSFPITFGCGTRLEIKSGGGGSEVQLVESGGGLVQPGGS |
| | | | | LKLSCAASGFTFNKYAMNWVRQAPGKGLEWVARIRSKY |
| | | | | NNYATYYADSVKDRFTISRDDSKNTAYLQMNNLKTEDTA |
| | | | | VYYCVRHGNFGNSYISYWAYWGQGTLVTVSSGGGGSGG |
| | | | | GGSGGGGSQ.TVVTQEPSLTVSPGGTVTLTCGSSTGAVTS |
| | | | | GNYPNWVQQKPGQAPRGLIGGTKFLAPGTPARFSGSLLG |
| | | | | GKAALTLSGVQPEDEAEYYCVLWYSNRWVFGGGTKLTVL |
| 183 | Anti- | artificial | aa | QVQMVQSGAEVKKHGASVKVSCKASGYTFTGYYMHWV |
| | Cldn18.2xCD3 | | | RQAPGQCLEWMGWINPNSGGTKYAQKFQGRVTMTRD |
| | Bispecific scFc | | | TSISTAYMELSRLRSDDTAVYYCARDRITVAGTYYYYGMD |
| | molecule | | | VWGQGTTVTVSSGGGGSGGGGSGGGGSDIQMTQSPSS |
| | CL-2xI2C-scFc | | | VSASVGDRVTITCRASQGVNNWLAWYQQKPGKAPKLLIY |
| | | | | TASSLQSGVPSRFSGSGSGTDFTLTIRSLQPEDFATYYCQQ |
| | | | | ANSFPITFGCGTRLEIKSGGGGSEVQLVESGGGLVQPGGS |
| | | | | LKLSCAASGFTFNKYAMNWVRQAPGKGLEWVARIRSKY |
| | | | | NNYATYYADSVKDRFTISRDDSKNTAYLQMNNLKTEDTA |
| | | | | VYYCVRHGNFGNSYISYWAYWGQGTLVTVSSGGGGSGG |
| | | | | GGSGGGGSQTVVTQEPSLTVSPGGTVTLTCGSSTGAVTS |
| | | | | GNYPNWVQQKPGQAPRGLIGGTKFLAPGTPARFSGSLLG |
| | | | | GKAALTLSGVQPEDEAEYYCVLWYSNRWVFGGGTKLTVL |
| | | | | GGGGDKTHTCPPCPAPELLGGPSVFLFPPKPKDTLMISRT |
| | | | | PEVTCVVVDVSHEDPEVKFNWYVDGVEVHNAKTKPCEE |
| | | | | QYGSTYRCVSVLTVLHQDWLNGKEYKCKVSNKALPAPIEK |
| | | | | TISKAKGQPREPQVYTLPPSREEMTKNQVSLTCLVKGFYPS |
| | | | | DIAVEWESNGQPENNYKTTPPVLDSDGSFFLYSKLTVDKS |
| | | | | RWQQGNVFSCSVMHEALHNHYTQKSLSLSPGKGGGGS |
| | | | | GGGGSGGGGSGGGGSGGGGSGGGGSDKTHTCPPCPAP |
| | | | | ELLGGPSVFLFPPKPKDTLMISRTPEVTCVVVDVSHEDPEV |
| | | | | KFNWYVDGVEVHNAKTKPCEEQYGSTYRCVSVLTVLHQD |
| | | | | WLNGKEYKCKVSNKALPAPIEKTISKAKGQPREPQVYTLPP |
| | | | | SREEMTKNQVSLTCLVKGFYPSDIAVEWESNGQPENNYK |
| | | | | TTPPVLDSDGSFFLYSKLTVDKSRWQQGNVFSCSVMHEA |
| | | | | LHNHYTQKSLSLSPGK |
| 184 | Anti-MUC17 | artificial | aa | GYYWS |
| | VH CDR1 | | | |
| | MU 8-B7 | | | |
| 185 | Anti-MUC17 | artificial | aa | DIDASGSTKYNPSLKS |
| | VH CDR2 | | | |
| | MU 8-B7 | | | |
| 186 | Anti-MUC17 | artificial | aa | KKYSTVWSYFDN |
| | VH CDR3 | | | |
| | MU 8-B7 | | | |
| 187 | Anti-MUC17 | artificial | aa | SGDKLGDKYAS |
| | VL CDR1 | | | |
| | MU 8-B7 | | | |
| 188 | Anti-MUC17 | artificial | aa | QDRKRPS |
| | VL CDR2 | | | |
| | MU 8-B7 | | | |
| 189 | Anti-MUC17 | artificial | aa | QAWGSSTAV |
| | VL CDR3 | | | |
| | MU 8-B7 | | | |
| 190 | Anti-MUC17 | artificial | aa | QVQLQQWGAGLLKPSETLSLTCAVYGGSFSGYYWSWIRQPPG |
| | VH | | | KCLEWIGDIDASGSTKYNPSLKSRVTISLDTSKNQFSLKLNSVTAA |
| | MU 8-B7 | | | DTAVYFCARKKYSTVWSYFDNWGQGTLVTVSS |
| 191 | Anti-MUC17 | artificial | aa | SYELTQPSSVSVPPGQTASITCSGDKLGDKYASWYQQKPG |
| | VL | | | QSPVLVIYQDRKRPSGVPERFSGSNSGNTATLTISGTQAM |
| | MU 8-B7 | | | DEADYYCQAWGSSTAVFGCGTKLTVL |
| 192 | bispecific | artificial | aa | QVQLQQWGAGLLKPSETLSLTCAVYGGSFSGYYWSWIR |
| | molecule | | | QPPGKCLEWIGDIDASGSTKYNPSLKSRVTISLDTSKNQFS |
| | MU 8-B7 x | | | LKLNSVTAADTAVYFCARKKYSTVWSYFDNWGQGTLVTV |
| | I2C0scFc | | | SSGGGGSGGGGSGGGGSSYELTQPSSVSVPPGQTASITCS |
| | | | | GDKLGDKYASWYQQKPGQSPVLVIYQDRKRPSGVPERFS |
| | | | | GSNSGNTATLTISGTQAMDEADYYCQAWGSSTAVFGCG |
| | | | | TKLTVLSGGGGSEVQLVESGGGLVQPGGSLKLSCAASGFT |
| | | | | FNKYAMNWVRQAPGKGLEWVARIRSKYNNYATYYADSV |
| | | | | KDRFTISRDDSKNTAYLQMNNLKTEDTAVYYCVRHGNFG |
| | | | | NSYISYWAYWGQGTLVTVSSGGGGSGGGGSGGGGSQT |
| | | | | VVTQEPSLTVSPGGTVTLTCGSSTGAVTSGNYPNWVQQK |
| | | | | PGQAPRGLIGGTKFLAPGTPARFSGSLLGGKAALTLSGVQ |
| | | | | PEDEAEYYCVLWYSNRWVFGGGTKLTVLGGGGDKTHTC |
| | | | | PPCPAPELLGGPSVFLFPPKPKDTLMISRTPEVTCVVVDVS |
| | | | | HEDPEVKFNWYVDGVEVHNAKTKPCEEQYGSTYRCVSVL |
| | | | | TVLHQDWLNGKEYKCKVSNKALPAPIEKTISKAKGQPREP |
| | | | | QVYTLPPSREEMTKNQVSLTCLVKGFYPSDIAVEWESNG |
| | | | | QPENNYKTTPPVLDSDGSFFLYSKLTVDKSRWQQGNVFS |
| | | | | CSVMHEALHNHYTQKSLSLSPGKGGGGSGGGGSGGGGS |
| | | | | GGGGSGGGGSGGGGSDKTHTCPPCPAPELLGGPSVFLFP |
| | | | | PKPKDTLMISRTPEVTCVVVDVSHEDPEVKFNWYVDGVE |
| | | | | VHNAKTKPCEEQYGSTYRCVSVLTVLHQDWLNGKEYKCK |
| | | | | VSNKALPAPIEKTISKAKGQPREPQVYTLPPSREEMTKNQ |
| | | | | VSLTCLVKGFYPSDIAVEWESNGQPENNYKTTPPVLDSDG |
| | | | | SFFLYSKLTVDKSRWQQGNVFSCSVMHEALHNHYTQKSL |
| | | | | SLSPGK |
| 193 | CDR-L1 of I2C | artificial | aa | GSSTGAVTSGNYPN |
| 194 | CDR-L2 of I2C | artificial | aa | GTKFLAP |
| 195 | CDR-L3 of I2C | artificial | aa | VLWYSNRWV |
| 196 | CDR-H1 of I2C | artificial | aa | KYAMN |
| 197 | CDR-H2 of I2C | artificial | aa | RIRSKYNNYATYYADSVKD |
| 198 | CDR-H3 of I2C | artificial | aa | HGNFGNSYISYWAY |
| 199 | VH of I2C | artificial | aa | EVQLVESGGGLVQPGGSLKLSCAASGFTFNKYAMNWVR |
| | | | | QAPGKGLEWVARIRSKYNNYATYYADSVKDRFTISRDDSK |
| | | | | NTAYLQMNNLKTEDTAVYYCVRHGNFGNSYISYWAYWG |
| | | | | QGTLVTVSS |
| 200 | VL of I2C | artificial | aa | QTVVTQEPSLTVSPGGTVTLTCGSSTGAVTSGNYPNWVQ |
| | | | | QKPGQAPRGLIGGTKFLAPGTPARFSGSLLGGKAALTLSG |
| | | | | VQPEDEAEYYCVLWYSNRWVFGGGTKLTVL |
| 201 | VH-VL of I2C | artificial | aa | EVQLVESGGGLVQPGGSLKLSCAASGFTFNKYAMNWVR |
| | | | | QAPGKGLEWVARIRSKYNNYATYYADSVKDRFTISRDDSK |
| | | | | NTAYLQMNNLKTEDTAVYYCVRHGNFGNSYISYWAYWG |
| | | | | QGTLVTVSSGGGGSGGGGSGGGGSQTVVTQEPSLTVSP |
| | | | | GGTVTLTCGSSTGAVTSGNYPNWVQQKPGQAPRGLIGG |
| | | | | TKFLAPGTPARFSGSLLGGKAALTLSGVQPEDEAEYYCVL |
| | | | | WYSNRWVFGGGTKLTVL |

The invention will be more fully understood by reference to the following examples. The examples should not, however, be construed as limiting the scope of the invention.

### EXAMPLES

### Example 1. Method of Measuring Protein Binding to Solid Surfaces

Bispecific antibody constructs were internally sourced. They were labeled with a fluorophore and purified after the labeling procedure.

Each measurement chamber contains a plastic coverslip. To measure the protein binding to solid surfaces, the measurement chambers with plastic coverslips on the bottom were incubated with a solution containing a fluorophore-protein (e.g., a fluorophore labeled antibody construct) first. Then the sample solution was aspirated out, the from the coverslips were rinsed and filled with buffer for imaging on a confocal microscope later. Fluorescence intensity as measured by the confocal microscope shows the binding of the bispecific antibody constructs to the coverslips. FIG. 1 shows the diagram of the experimental set-up.

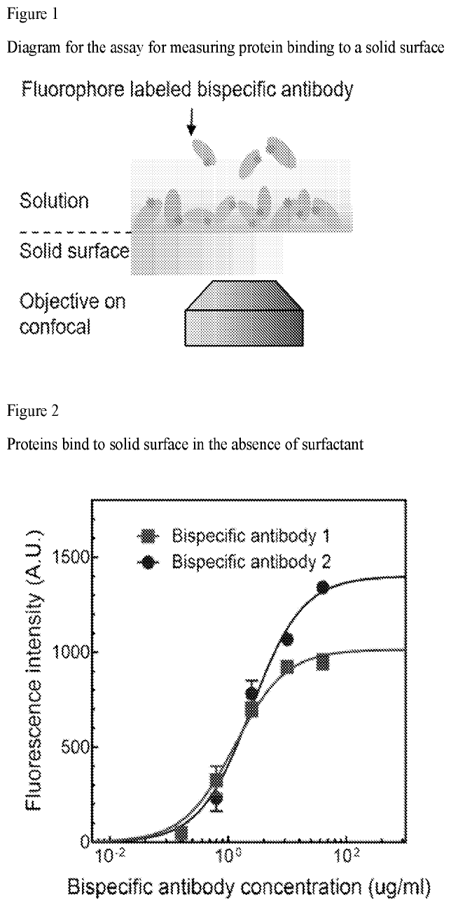

FIG. 2 shows titrations of two fluorophore-labeled antibody constructs binding to the solid surfaces (e.g., coverslips), separately, in the absence of surfactant. The fluorescence intensities of bound fluorophore-labeled antibody constructs were measured by the confocal xy scans of the surface.

### Example 2. Treatment of Solid Surfaces with Surfactants Preventing Protein Binding to the Surfaces

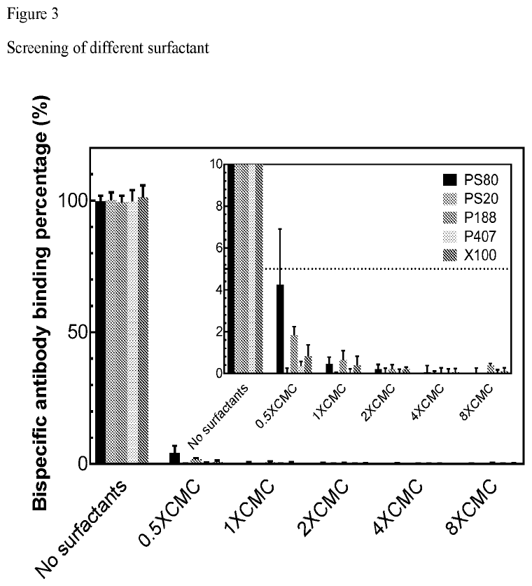

Several surfactants were used at different folds of their respective CMC to determine the effectiveness of each surfactant at preventing bispecific antibody construct binding to solid surface. In this study, a solution containing surfactants was incubated with the surface first, then the fluorophore labeled antibody constructs were added and incubated. After that, the solution was aspirated out, the surface was rinsed and filled with buffer for imaging on a confocal microscope. The results are shown in FIG. 3. In the figure, the first group of bars are the bispecific antibody binding to the surface without any surfactants. These were served as benchmark and all the following groups of data were compared to those. From the 2nd to the last group are the relative percentages of protein bound to the surfaces pre-treated with different surfactants at different folds of their distinguish CMCs. The insert is a zoom-in for the lower region of the graph. PS 80, PS 20, P188, P407 and Triton X-100 were investigated.

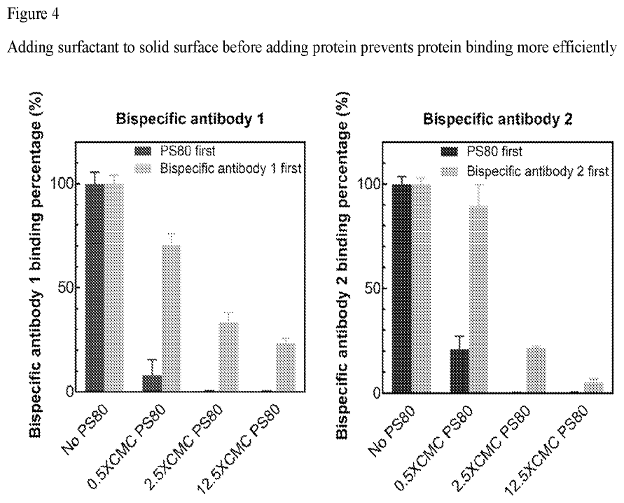

The order of adding the surfactants and the antibodies to the surface was tested. For bispecific antibodies 1 & 2, two orders were tested: in the first one, a surfactant-containing solution was added to the surface before adding the antibodies to the surface; while the other one was vice versa. The results were shown in FIG. 4 (left is antibody 1 and right is antibody 2). For both graphs, the first group of bars are the bispecific antibody binding to the surface without any surfactants. These were served as benchmark and all the following groups of data were compared to those. From the 2nd to the last group are the relative percentages of the antibodies bound to the surfaces pre-treated with PS 80 at different folds of its CMC. The surfactants effectively prevented the proteins binding to the surfaces.

### Example 3. Incompatibility of Certain Surfactants and the Plasticizers Used in Plastic IV Components

Baxter Viaflex PVC-DEHP IV bags pre-filled with saline diluent were used for the study. Surfactants polysorbate 80 (PS80), polysorbate 20 (PS20), poloxamer 188 (P188), poloxamer 407 (P407) and Triton X-100 were used in the study. Different amounts of different surfactants were incubated with the bags at 25° C. for 24 hrs or 48 hrs. Then the bags were sampled and analyzed by reversed-phase ultra-high pressure liquid chromatography (RP-UHPLC) and detected by an UV detector. The mobile phase A & B are 0.1% trifluoroacetic acid (TFA) in DI-water and 0.1% TFA in acetonitrile. The gradient is listed in the Table 3 below. Flow rate is 0.6 ml/min. For quantification, a standard curve of DEHP was established under the same conditions.

TABLE 3
RP-UPLC gradient

| Time | Flow | | |
|---|---|---|---|
| (min) | (mL/min) | % A | % B |
| 0.0 | 0.6 | 95 | 5 |
| 1.5 | 0.6 | 5 | 95 |
| 3.1 | 0.6 | 5 | 95 |
| 3.2 | 0.6 | 95 | 5 |
| 4.0 | 0.6 | 95 | 5 |

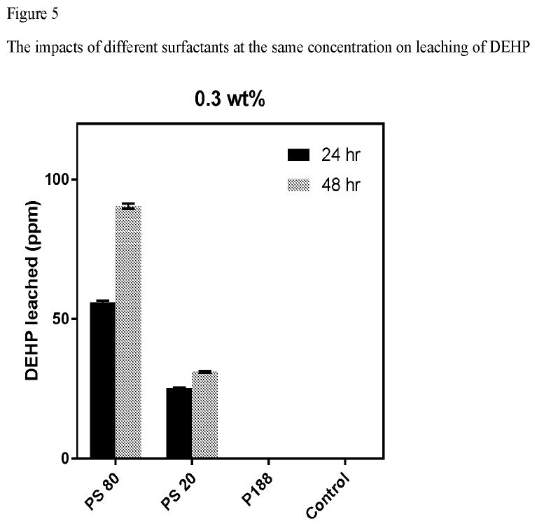

PS 80, PS 20 and P188 were compared at 0.3 wt % in saline in PVC-DEHP IV bags for 24 hrs and 48 hrs at 25° C. The results are shown in FIG. 5. PS80 & PS20 caused significant leaching of DEHP from PVC-DEHP IV bags, while P188 didn't cause any leaching (FIG. 5). Saline only was used as a control.

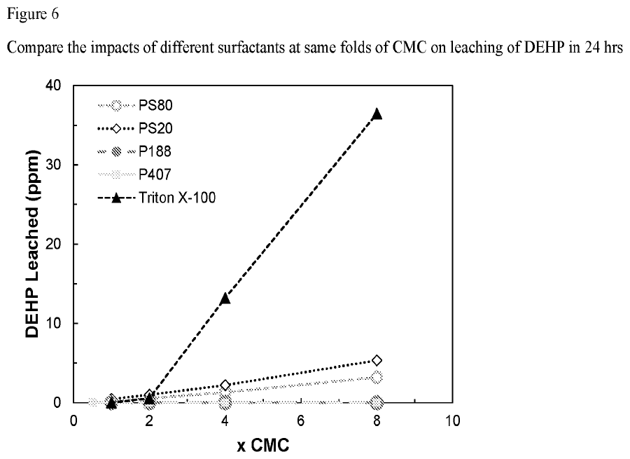

PS 80, PS 20, P188, P407 and Triton X-100 were compared at different folds of their respective CMC by incubating in saline in PVC-DEHP IV bags for 24 hrs at 25° C. The amount of leached DEHP was plotted as a function of the folds of CMC in FIG. 6. Clearly, polysorbates extract certain amounts of DEHP while poloxamers don't. And the amounts of DEHP leached are correlated with the amounts of surfactants used.

What is claimed:

1. An aqueous composition comprising a bispecific antibody construct at a concentration of between about 0.001 μg/ml and about 100 μg/ml and a surfactant at a concentration of at least about 0.25× of the critical micelle concentration (CMC) of the surfactant, wherein the bispecific antibody construct comprises a first binding domain that binds to a target cell surface antigen, a second binding domain that binds to human CD3 on the surface of a T cell, and optionally, a third domain comprising, in an amino to carboxyl order, hinge-CH2 domain-CH3 domain-linker-hinge-CH2 domain-CH3 domain, wherein the second binding domain comprises a polypeptide having the sequence of SEQ ID NO: 201.

2. The composition of claim 1, wherein the bispecific antibody construct is present at a concentration of between about 0.001 μg/ml and about 50 μg/ml.

3. The composition of claim 1 or 2, wherein the bispecific antibody construct is present at a concentration of between about 0.01 μg/ml to about 50 μg/ml, or between 0.1 μg/ml to about 50 μg/ml, or 0.1 μg/ml to about 10 μg/ml, or 1 μg/ml to about 10 μg/ml.

4. The composition of any one of claims 1-3, wherein the surfactant is a polysorbate, a poloxamer or triton x-100.

5. The composition of any one of claims 1-4, wherein the surfactant is polysorbate 80, polysorbate 60, polysorbate 40, polysorbate 20, or Triton X-100.

6. The composition of any one of claims 1-4, wherein the surfactant is poloxamer 188 or poloxamer 407.

7. The composition of any one of claims 1-6, wherein the surfactant is present at a concentration of between about 0.25× and about 20× of the CMC, or between about 0.25× and about 10× of the CMC of the surfactant.

8. The composition of any one of claims 1-7, wherein the composition further comprising a salt, an amino acid, a saccharide or saccharide derivative, or combinations thereof.

9. The composition of claim 8, wherein the composition further comprises a buffer or a preservative.

10. The composition of claim 8 or 9, wherein the pH of the composition is between about 3.5 and about 7.5.

11. The composition of claim 10, wherein the pH of the composition is between about 4.2 and about 7.0.

12. The composition of any one of claims 8-11, wherein the salt is NaCl.

13. The composition of any one of claims 8-12, wherein the saccharide or saccharide derivative is a monosaccharide, a disaccharide, a cyclic polysaccharide or a sugar alcohol.

14. The composition of claims 8-13, wherein the saccharide is sucrose, trehalose, mannitol or sorbitol.

15. The composition of any one of claims 8-14, wherein the amino acid is lysine.

16. The composition of any one of claims 9-15, wherein the buffer is an acetate buffer, a glutamate buffer, a citrate buffer, a succinate buffer, a tartrate buffer, a fumarate buffer, a maleate buffer, a histidine buffer, or phosphate buffer.

17. The composition of any one of claims 1-16, wherein each of the first and second binding domains of the bispecific antibody construct comprises a VH region and a VL region.

18. The composition of any one of claims 1-17, wherein the bispecific antibody construct is a single chain antibody construct.

19. The composition of any one of claims 1-18, wherein the bispecific antibody construct comprises a polypeptide having the amino acid sequence selected from SEQ ID NO: 11, SEQ ID NO: 13, SEQ ID NO: 15, SEQ ID NO: 24, SEQ ID NO: 25, SEQ ID NO: 47, SEQ ID NO: 48, SEQ ID NO: 66, SEQ ID NO: 67, SEQ ID NO: 76, SEQ ID NO: 78, SEQ ID NO: 87, SEQ ID NO: 88, SEQ ID NO: 108, SEQ ID NO: 109, SEQ ID NO: 110, SEQ ID NO: 165, SEQ ID NO: 166, SEQ ID NO: 167, SEQ ID NO: 176, SEQ ID NO: 178, and SEQ ID NO: 192.

20. The composition of any one of claims 1-19, wherein the composition is a pharmaceutical composition.

21. The composition of any one of claims 1-20, wherein the composition is contained in a plastic container.

22. The composition of claim 21, wherein the container is made of a material comprising polyolefin, PVC, EVA, or polyurethane.

23. The composition of claim 22, wherein the container is made of a material comprising PVC and wherein the PVC is substantially free of DEHP or TOTM.

24. The composition of any one of claims 21-23, wherein the container is an IV bag or IV tube.

25. A pharmaceutical preparation comprising an aqueous pharmaceutical composition contained inside a container, wherein the aqueous pharmaceutical composition comprising:
a) a bispecific antibody construct at a concentration of between about 0.001 μg/ml and about 100 μg/ml, and
b) a surfactant at a concentration of at least about 0.25× of CMC of the surfactant, wherein the surfactant has an HLB value of at least 20.

26. The pharmaceutical preparation of claim 25, wherein the aqueous pharmaceutical composition comprises the bispecific antibody construct at a concentration of between about 0.001 μg/ml and about 50 μg/ml.

27. The pharmaceutical preparation of claim 25 or 26, wherein the aqueous pharmaceutical composition comprises the surfactant is at a concentration of between about 0.25× and about 20× of the CMC or between about 0.25× and about 10× of the CMC, of the surfactant.

28. The pharmaceutical preparation of any one of claims 25-27, wherein the aqueous pharmaceutical composition further comprising a salt, a buffer, an amino acid, a saccharide or saccharide derivative, or combinations thereof.

29. The pharmaceutical preparation of any one of claims 25-28, wherein the aqueous pharmaceutical composition has a pH of between about 4.2 and about 7.0.

30. The pharmaceutical preparation of any one of claims 25-29, wherein the container is made of a material comprising polyolefin, PVC, EVA or polyurethane.

31. The pharmaceutical preparation of any one of claims 25-30, wherein the bispecific antibody construct comprises a polypeptide having the amino acid sequences selected from SEQ ID NO: 11, SEQ ID NO: 13, SEQ ID NO: 15, SEQ ID NO: 24, SEQ ID NO: 25, SEQ ID NO: 47, SEQ ID NO: 48, SEQ ID NO: 66, SEQ ID NO: 67, SEQ ID NO: 76, SEQ ID NO: 78, SEQ ID NO: 87, SEQ ID NO: 88, SEQ ID NO: 108, SEQ ID NO: 109, SEQ ID NO: 110, SEQ ID NO: 165, SEQ ID NO: 166, SEQ ID NO: 167, SEQ ID NO: 176, SEQ ID NO: 178, and SEQ ID NO: 192.

32. A method of administering a bispecific antibody construct to a patient comprising:
a) preparing an aqueous pharmaceutical composition in a container, wherein the aqueous pharmaceutical composition comprises the bispecific antibody construct at a concentration of between about 0.001 μg/ml and about 100 μg/ml and a surfactant at a concentration of at least about 0.25× of CMC of the surfactant, and
b) administering the aqueous pharmaceutical composition to the patient.
wherein the bispecific antibody construct comprises a polypeptide having the amino acid sequence selected from SEQ ID NO: 11, SEQ ID NO: 13, SEQ ID NO: 15, SEQ ID NO: 24, SEQ ID NO: 25, SEQ ID NO: 47, SEQ ID NO: 48, SEQ ID NO: 66, SEQ ID NO: 67, SEQ ID NO: 76, SEQ ID NO: 78, SEQ ID NO: 87, SEQ ID NO: 88, SEQ ID NO: 108, SEQ ID NO: 109, SEQ ID NO: 110, SEQ ID NO: 165, SEQ ID NO: 166, SEQ ID NO: 167, SEQ ID NO: 176, SEQ ID NO: 178, and SEQ ID NO: 192.

33. The method of claim 32, wherein the aqueous pharmaceutical composition comprises the bispecific antibody construct is at a concentration of between about 0.001 μg/ml and about 50 μg/ml.

34. The method of claim 32 or 33, wherein the aqueous pharmaceutical composition comprises the surfactant is at a concentration of between about 0.25× and about 20× of the CMC or between about 0.25× and about 10× of the CMC, of the surfactant.

35. The method of any one of claims 32-34, wherein the surfactant is polysorbate 80, polysorbate 60, polysorbate 40, polysorbate 20, poloxamer 188, poloxamer 407, or Triton X-100.

36. The method of any one of claims 32-35, wherein the aqueous pharmaceutical composition further comprising one or more selected from a salt, a buffer, an amino acid, a saccharide, and a preservative.

37. The method of any one of claims 32-36, wherein the aqueous pharmaceutical composition has a pH of between about 4.2 and about 7.0.

38. The method of any one of claims 32-37, wherein the container is made of a material comprising polyolefin, PVC, EVA, or polyurethane.

39. The method of any one of claims 32-38, wherein the surfactant is polysorbate 80, polysorbate 60, polysorbate 40, or polysorbate 20, and wherein the container is made of a material comprising PVC that is substantially free of DEHP or TOTM.

40. The method of any one of claims 32-39, wherein the aqueous pharmaceutical composition is prepared by diluting a first composition comprising the bispecific antibody construct with a suitable aqueous solution.

41. The method of claim 40, wherein the first composition is a liquid composition comprising the bispecific antibody construct.

42. The method of claim 40, wherein the first composition is a liquid composition reconstituted from a lyophilized composition comprising the bispecific antibody construct.

43. The method of any one of claims 41-42, wherein the suitable solution comprises the surfactant at a concentration of at least about 0.25× of CMC of the surfactant.

44. The method of any one of claims 40-43, wherein the aqueous pharmaceutical composition is prepared by adding the suitable aqueous solution into the container followed by adding an appropriate amount of the first composition into the container.

45. The method of any one of claims 32-44, wherein the patient is a cancer patient.

46. The method of any one of claims 32-45, wherein the administration is IV administration.

47. The pharmaceutical preparation of any one of claims 25-31, wherein the surfactant is poloxamer 188 or poloxamer 407.

48. The composition of claim 19, the pharmaceutical preparation of claim 31 or the method of claim 32, wherein the bispecific antibody construct comprises a polypeptide having the amino acid sequence of SEQ ID NO: 13, SEQ ID NO: 15, SEQ ID NO: 25, SEQ ID NO: 48, SEQ ID NO: 67, SEQ ID NO: 78, SEQ ID NO: 88, SEQ ID NO: 109, SEQ ID NO: 110, SEQ ID NO: 165, SEQ ID NO: 166, SEQ ID NO: 167, SEQ ID NO: 178 or SEQ ID NO: 192.
